(19) United States
(12) Patent Application Publication
(10) Pub. No.: US 2025/0329015 A1
(43) Pub. Date: Oct. 23, 2025

(54) SYSTEMS, METHODS, AND DEVICES FOR PLAQUE ANALYSIS, VESSEL AND FLUID FLOW ANALYSIS, AND/OR RISK DETERMINATION OR PREDICTION THEREOF

(71) Applicant: Cleerly, Inc., Denver, CO (US)

(72) Inventors: James K. Min, Chapel Hill, NC (US); Chung Chan, Northbrook, IL (US); James P. Earls, Alexandria, VA (US)

(21) Appl. No.: 19/186,433

(22) Filed: Apr. 22, 2025

Related U.S. Application Data
(60) Provisional application No. 63/637,823, filed on Apr. 23, 2024.
(60) Provisional application No. 63/637,829, filed on Apr. 23, 2024.
(60) Provisional application No. 63/683,136, filed on Aug. 14, 2024.
(60) Provisional application No. 63/694,668, filed on Sep. 13, 2024.
(60) Provisional application No. 63/697,951, filed on Sep. 23, 2024.
(60) Provisional application No. 63/698,937, filed on Sep. 25, 2024.
(60) Provisional application No. 63/708,078, filed on Oct. 16, 2024.

(51) Int. Cl. G06T 7/00; A61B 6/00; A61B 6/03; A61B 6/50; G06T 7/246; G06T 7/60; G16H 50/20

(52) U.S. Cl. CPC G06T 7/0012; A61B 6/032; A61B 6/481; A61B 6/504; A61B 6/507; A61B 6/5217; G06T 7/251; G06T 7/60; G16H 50/20; G06T 2207/10081; G06T 2207/20081; G06T 2207/20084; G06T 2207/30048; G06T 2207/30096; G06T 2207/30104

(57) ABSTRACT

This disclosure relates to systems, methods, and devices for plaque analysis, vessel and fluid flow analysis, and/or risk determination or prediction thereof. Some embodiments relate to determining fractional flow reserve (FFR) values. Some embodiments relate to determining FFR values using 3D-printed models. Some embodiments relate to identifying thin cap fibroatheroma based on analysis of computed tomography (CT) imaging. Some embodiments relate adjusting calcified plaque thresholds for CT images to address effects of calcium blooming.

### PRIORITY APPLICATIONS

This application claims priority to U.S. Provisional Application No. 63/708,078 filed Oct. 16, 2024, U.S. Provisional Application No. 63/694,668 filed Sep. 13, 2024, U.S. Provisional Application No. 63/698,937 filed Sep. 25, 2024, U.S. Provisional Application No. 63/697,951 filed Sep. 23, 2024, U.S. Provisional Application No. 63/683,136 filed Aug. 14, 2024, U.S. Provisional Application No. 63/637,829 filed Apr. 23, 2024, and U.S. Provisional Application No. 63/637,823 filed Apr. 23, 2024, each of which are incorporated herein by reference. Any and all applications for which a foreign or domestic priority claim is identified in the Application Data Sheet as filed with the present application are hereby incorporated by reference under 37 CFR 1.57.

### BACKGROUND

Fractional flow reserve and other metrics can be used to evaluate cardiac health. However, there are significant challenges with current approaches. Accordingly, there is a need for improved approaches.

### BRIEF DESCRIPTION OF THE DRAWINGS

A better understanding of the devices and methods described herein will be appreciated upon reference to the following description in conjunction with the accompanying drawings, wherein:

FIG. 1 depicts a schematic of an example of an embodiment of a system that includes a processing system configured to characterize coronary plaque.

FIG. 2 is a schematic illustrating an example of a heart muscle and its coronary arteries.

FIG. 3 illustrates an example of a set of images generated from scanning along a coronary artery, including a selected image of a portion of a coronary artery, and how image data may correspond to a value on the Hounsfield Scale.

FIG. 4 is a block diagram that illustrates a computer system upon which various embodiments may be implemented.

FIG. 5A is a block diagram that illustrates an example of a system and/or process (both referred to here as a “system” for ease of reference) for identifying features and/or risk information of a patient using AI/ML based on non-invasively obtained medical images of the patient and/or patient information.

FIG. 5B is a schematic illustrating an example of a NN that makes determinations about characteristics of a (current) patient based on inputs that include medical images.

FIG. 5C depicts an example of a process in a flow diagram for training an artificial intelligence or machine learning model.

FIG. 5D illustrates an example of a process for training and using an A I/ML model.

FIG. 6 illustrates an example computer system on which some embodiments can be performed.

FIG. 7 is a flowchart that illustrates an example process for generating data for a machine learning model and training a machine learning model according to some implementations.

FIG. 8 is a flow chart that illustrates an example process for adjusting a machine learning model according to some embodiments.

FIG. 9 is a flowchart that illustrates an example process for determining systematic offsets that can be applied to the outputs of a machine learning model according to some embodiments.

FIG. 10 is a flowchart that illustrates an example physics-based, per-vessel approach for mapping FFR 3D and FFR values according to some embodiments.

FIG. 11 is a flowchart that illustrates an example physics-based, per-segment process for mapping FFR3D and FFR values according to some embodiments.

FIG. 12 is a flowchart that illustrates an example physics-based, per-unit-length process for mapping FFR3D and FFR values according to some embodiments.

FIG. 13 is a flowchart that illustrates an example process for training a machine learning model to generate FFR3D values according to some embodiments.

FIG. 14 is a flowchart that illustrates an example process that combines physics-based and anatomical-based approaches according to some embodiments.

FIG. 15 is a drawing that illustrates idealized stenoses and pressure within a vessel at various locations.

FIGS. 16A-16C illustrate pressure drops as a function of blood flow rates with various percent diameter stenosis.

FIG. 16D-16F illustrate pressure ratios at varying flow rates with various percent diameter stenoses.

FIG. 17 is a diagram that schematically illustrates stenosis and fluid flow.

FIGS. 18A-18C illustrate graphs of pressure drop as a function of blood flow rate, pressure ratio as a function of blood flow rate, and distal pressure as a function of CFR.

FIG. 19 is a flowchart that illustrates an example process for training an algorithm to predict FFR values along a coronary tree according to some embodiments.

FIG. 20 is a flowchart the illustrates an example process for training an algorithm to predict FFR based on patient-specific geometry and pressure pullback gradient (PPG) curves according to some embodiments.

FIG. 21 is a flowchart that illustrates an example multi-algorithm process according to some embodiments.

FIG. 22 is a drawing that illustrates an example process for developing an algorithm for FFR estimation/calculation using patient-specific geometry/anatomic inputs to estimate quadratic relationships and PPG curves according to some embodiments.

FIG. 23 is a plot that illustrates prescribed flow reserve concepts according to some embodiments.

FIG. 24 is a flowchart that illustrates an example process for training and deploying a machine learning model according to some implementations.

FIG. 25 is a flowchart that illustrates an example process for combining OD CFD calculations and 3D printing according to some implementations.

FIG. 26 illustrates examples of data that can be used for training a machine learning model according to some embodiments.

FIG. 27 is a flowchart that illustrates an example process for evaluating a subject using reduced order computation fluid dynamics data according to some embodiments.

FIG. 28 is a flowchart of an example method for determining and checking the correctness of vessel labeling according to some embodiments.

FIG. 29 is a flowchart of an example method for determining and checking the correctness of vessel labeling according to some embodiments.

FIG. 30 is a flowchart that illustrates an example process for determining calcified plaque thresholds according to some embodiments.

FIG. 31 is a flowchart that illustrates another example process for determined calcified plaque thresholds according to some embodiments.

FIG. 32 is a flowchart that illustrates an example process for training and deploying a machine learning model for calcified plaque characterization according to some embodiments.

FIG. 33 is a flowchart that illustrates an example process for creating and/or updating a calcified plaque threshold table according to some embodiments.

FIG. 34 is a drawing that illustrates calcified plaque determination using IVUS according to some embodiments.

FIG. 35 is a diagram that illustrates example correlations between calcified plaque as determined by IVUS and CCTA for different calcified plaque thresholds.

FIG. 36 is a plot that illustrates R² values at different calcified plaque thresholds for different peak kilovoltages (kV p) according to some embodiments.

FIG. 37 illustrates box plots of calcified plaque index, calcified plaque length, and calcified plaque maximum angle as determined by CT and IVUS according to some embodiments.

FIG. 38 is a table that compares calcified plaque index as determined by CT and IVUS according to some embodiments.

FIG. 39 is a table that compares calcified plaque length as determined by CT and IVUS according to some embodiments.

FIG. 40 is a table that shows examples of comparisons of calcified plaque angle as determined by CT and by IV US according to some embodiments.

FIG. 41 is a table that shows examples of sensitivity, specificity, positive predictive value (PPV), negative predictive value (NPV), and accuracy at various calcified plaque thresholds according to some embodiments.

FIG. 42 is a receiver operating characteristic plot showing various calcified plaque thresholds in comparison with IVUS measurements according to some embodiments.

FIG. 43 shows an example OCT image with regions of calcium according to some embodiments.

FIG. 44 is a drawing that illustrates calcified plaque (blue) and non-calcified plaque at various calcified plaque thresholds according to some embodiments.

FIG. 45 is a plot that shows root mean squared error (RMSE) as a function of calcified plaque threshold according to some embodiments.

FIG. 46A is a flowchart that shows an example process for determining a threshold HU value for differentiating between LAP and other non-calcified plaque according to some implementations.

FIG. 46B is a flowchart that illustrates an example process for determining a LAP threshold according to some implementations.

FIG. 47 is a flowchart that illustrates an example process for determining a threshold HU value for identifying TCFA according to some implementations.

FIG. 48 is a flowchart that illustrates an example process for identifying TCFA in CCTA images using machine learning according to some implementations.

FIGS. 49A and 49B schematically illustrate examples of CCTA images that do not show TCFA and do show TCFA.

FIGS. 50A and 50B schematically illustrate an example of a CCTA image with different cutoff thresholds for LAP.

FIG. 51 illustrates an example of the separation between thin cap and thick cap fibroatheromas at different Hounsfield unit thresholds according to some implementations.

FIG. 52 is a drawing that illustrates an example of thin (<65 micrometers) and thick (>65 micrometers) cap fibroatheromas.

FIG. 53 is a drawing that illustrates identified LAP in CCTA images at various LAP thresholds.

FIG. 54 is a flowchart that illustrates an example process for training a machine learning model to determine low attenuation plaque thresholds according to some embodiments.

FIG. 55 is a flowchart that illustrates an example process for determining the presence and/or extent of thin cap fibroatheroma.

FIG. 56 is a flowchart that illustrates an example process for determining presence and vulnerability of TCFA according to some implementations.

### DETAILED DESCRIPTION

Although several embodiments, examples, and illustrations are disclosed below, it will be understood by those of ordinary skill in the art that the inventions described herein extend beyond the specifically disclosed embodiments, examples, and illustrations and includes other uses of the inventions and obvious modifications and equivalents thereof. Embodiments of the inventions are described with reference to the accompanying figures, wherein like numerals refer to like elements throughout. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

“Plaque” or “a region of plaque” or “one or more regions of plaque” may be referred to simply as “plaque” for ease of reference unless otherwise indicated, explicitly or by context. For example, in some embodiments, the systems, devices, and methods described herein are related to plaque analysis based on one or more of distance between plaque (e.g., coronary plaque) and a vessel wall, distance between plaque and a lumen wall, length along longitudinal axis of plaque, length along latitudinal axis of plaque, area or volume of low density non-calcified plaque, area or volume of non-calcified plaque, area or volume of calcified plaque, area or volume of total plaque, a ratio(s) between one or more of area or volume of low density non-calcified plaque, area or volume of non-calcified plaque, area or volume of calcified plaque, or area or volume of total plaque, and one or more of area or volume of low density non-calcified plaque, area or volume of non-calcified plaque, area or volume of calcified plaque, or area or volume of total plaque, embeddedness of low density non-calcified plaque, non-calcified plaque, calcified plaque, total plaque, or any plaque, and/or the like. In some embodiments, the systems, devices, and methods described herein are configured to determine a risk of coronary artery disease (CAD) and/or major adverse cardiovascular event (MACE), such as myocardial infarction (MI), based on one or more plaque analyses described herein. In some embodiments, the systems, devices, and methods described herein are configured to generate a proposed treatment and/or graphical representation based on the determined risk of CAD and/or one or more plaque analyses described herein.

In some embodiments, the systems, methods, and devices described herein are configured to analyze one or more coronary computed tomography angiography (CCTA) images to identify one or more high-risk plaques or atherosclerosis. In some embodiments, high-risk plaque or atherosclerosis can be identified when one or more high-risk factors are present, including for example high volume, burden, composition, density, radiodensity, and/or the like. In some embodiments, high-risk plaque or atherosclerosis can be identified on the patient level and/or at the lesion level.

In some embodiments, the systems, methods, and devices described herein can be configured to analyze total plaque volume for a patient and/or presence and/or prevalence or extent of high-risk plaques. For example, in some embodiments, high-risk plaques can be identified based on low attenuation, low material density, low radiodensity, and/or high lesion-level plaque volume. In some embodiments, the systems, methods, and devices described herein can be configured to determine or generate a lesion-level risk score. In some embodiments, a lesion-level risk score can be configured to be used to identify one or more local lesions that have a poor prognosis and/or that comprise a high or relatively high risk of becoming a culprit lesion at the time of a future MI.

In some embodiments, the system can be configured to characterize a particular region of plaque as high-risk or low density non-calcified plaque when the radiodensity of an image pixel or voxel corresponding to that region of plaque is between about −189 and about 30 Hounsfield units (HU). In some embodiments, the system can be configured to characterize a particular region of plaque as non-calcified plaque when the radiodensity of an image pixel or voxel corresponding to that region of plaque is between about 31 and about 350 HU. In some embodiments, the system can be configured to characterize a particular region of plaque as calcified plaque when the radiodensity of an image pixel or voxel corresponding to that region of plaque is between about 351 and about 2500 HU. In some embodiments, the lower and/or upper Hounsfield unit boundary threshold for determining whether a plaque corresponds to one or more of low density non-calcified plaque, non-calcified plaque, and/or calcified plaque can be about −1000 HU, about −900 HU, about −800 HU, about −700 HU, about −600 HU, about −500 HU, about −400 HU, about −300 HU, about −200 HU, about −190 HU, about −180 HU, about −170 HU, about −160 HU, about −150 HU, about −140 HU, about −130 HU, about −120 HU, about −110 HU, about −100 HU, about −90 HU, about −80 HU, about −70 HU, about −60 HU, about −50 HU, about −40 HU, about −30 HU, about −20 HU, about −10 HU, about 0 HU, about 10 HU, about 20 HU, about 30 HU, about 40 HU, about 50 HU, about 60 HU, about 70 HU, about 80 HU, about 90 HU, about 100 HU, about 110 HU, about 120 HU, about 130 HU, about 140 HU, about 150 HU, about 160 HU, about 170 HU, about 180 HU, about 190 HU, about 200 HU, about 210 HU, about 220 HU, about 230 HU, about 240 HU, about 250 HU, about 260 HU, about 270 HU, about 280 HU, about 290 HU, about 300 HU, about 310 HU, about 320 HU, about 330 HU, about 340 HU, about 350 HU, about 360 HU, about 370 HU, about 380 HU, about 390 HU, about 400 HU, about 410 HU, about 420 HU, about 430 HU, about 440 HU, about 450 HU, about 460 HU, about 470 HU, about 480 HU, about 490 HU, about 500 HU, about 510 HU, about 520 HU, about 530 HU, about 540 HU, about 550 HU, about 560 HU, about 570 HU, about 580 HU, about 590 HU, about 600 HU, about 700 HU, about 800 HU, about 900 HU, about 1000 HU, about 1100 HU, about 1200 HU, about 1300 HU, about 1400 HU, about 1500 HU, about 1600 HU, about 1700 HU, about 1800 HU, about 1900 HU, about 2000 HU, about 2100 HU, about 2200 HU, about 2300 HU, about 2400 HU, about 2500 HU, about 2600 HU, about 2700 HU, about 2800 HU, about 2900 HU, about 3000 HU, about 3100 HU, about 3200 HU, about 3300 HU, about 3400 HU, about 3500 HU, and/or about 4000 HU.

### INTRODUCTION

Coronary heart disease affects over 17.6 million Americans. The current trend in treating cardiovascular health issues is generally two-fold. First, physicians generally review a patient's cardiovascular health from a macro level, for example, by analyzing the biochemistry or blood content or biomarkers of a patient to determine whether there are high levels of cholesterol elements in the bloodstream of a patient. In response to high levels of cholesterol, some physicians will prescribe one or more drugs, such as statins, as part of a treatment plan in order to decrease what is perceived as high levels of cholesterol elements in the bloodstream of the patient.

The second general trend for currently treating cardiovascular health issues involves physicians evaluating a patient's cardiovascular health through the use of angiography to identify large blockages in various arteries of a patient. In response to finding large blockages in various arteries, physicians in some cases will perform an angioplasty procedure wherein a balloon catheter is guided to the point of narrowing in the vessel. After properly positioned, the balloon is inflated to compress or flatten the plaque or fatty matter into the artery wall and/or to stretch the artery open to increase the flow of blood through the vessel and/or to the heart. In some cases, the balloon is used to position and expand a stent within the vessel to compress the plaque and/or maintain the opening of the vessel to allow more blood to flow. About 500,000 heart stent procedures are performed each year in the United States.

However, a recent federally funded $100 million study calls into question whether the current trends in treating cardiovascular disease are the most effective treatment for all types of patients. The recent study involved over 5,000 patients with moderate to severe stable heart disease from 320 sites in 37 countries and provided new evidence showing that stents and bypass surgical procedures are likely no more effective than drugs combined with lifestyle changes for people with stable heart disease. Accordingly, it may be more advantageous for patients with stable heart disease to forgo invasive surgical procedures, such as angioplasty and/or heart bypass, and instead be prescribed heart medicines, such as statins, and certain lifestyle changes, such as regular exercise. This new treatment regimen could affect thousands of patients worldwide. Of the estimated 500,000 heart stent procedures performed annually in the United States, it is estimated that a fifth of those are for people with stable heart disease. It is further estimated that 25% of the estimated 100,000 people with stable heart disease, or roughly 23,000 people, are individuals that do not experience any chest pain. Accordingly, over 20,000 patients annually could potentially forgo invasive surgical procedures or the complications resulting from such procedures.

To determine whether a patient should forego invasive surgical procedures and opt instead for a drug regimen and/or to generate a more effective treatment plan, it can be important to more fully understand the cardiovascular disease of a patient. Specifically, it can be advantageous to better understand the arterial vessel health of a patient. For example, it is helpful to understand whether plaque build-up in a patient is mostly fatty matter build-up or mostly calcified matter build-up, because the former situation may warrant treatment with heart medicines, such as statins, whereas in the latter situation a patient should be subject to further periodic monitoring without prescribing heart medicine or implanting any stents. However, if the plaque build-up is significant enough to cause severe stenosis or narrowing of the arterial vessel such that blood flow to heart muscle might be blocked, then an invasive angioplasty procedure to implant a stent may likely be required because heart attack or sudden cardiac death (SCD) could occur in such patients without the implantation of a stent to enlarge the vessel opening. Sudden cardiac death is one of the largest causes of natural death in the United States, accounting for approximately 325,000 adult deaths per year and responsible for nearly half of all deaths from cardiovascular disease. For males, SCD is twice as common as compared to females. In general, SCD strikes people in the mid-30 to mid-40 age range. In over 50% of cases, sudden cardiac arrest occurs with no warning signs.

With respect to the millions suffering from heart disease, there is a need to better understand the overall health of the artery vessels within a patient beyond just knowing the blood chemistry or content of the blood flowing through such artery vessels. For example, in some embodiments of systems, devices, and methods disclosed herein, arteries with “good” or stable plaque or plaque comprising hardened calcified content are considered non-life threatening to patients whereas arteries containing “bad” or unstable plaque or plaque comprising fatty material are considered more life threatening because such bad plaque may rupture within arteries, thereby releasing such fatty material into the arteries. Such a fatty material release in the blood stream can cause inflammation that may result in a blood clot. A blood clot within an artery can prevent blood from traveling to heart muscle thereby causing a heart attack or other cardiac event. Further, in some instances, it is generally more difficult for blood to flow through fatty plaque buildup than it is for blood to flow through calcified plaque build-up. Therefore, there is a need for better understanding and analysis of the arterial vessel walls of a patient.

Further, while blood tests and drug treatment regimens are helpful in reducing cardiovascular health issues and mitigating against cardiovascular events (for example, heart attacks), such treatment methodologies are not complete or perfect in that such treatments can misidentify and/or fail to pinpoint or diagnose significant cardiovascular risk areas. For example, the mere analysis of the blood chemistry of a patient will not likely identify that a patient has artery vessels having significant amounts of fatty deposit material bad plaque buildup along a vessel wall. Similarly, an angiogram, while helpful in identifying areas of stenosis or vessel narrowing, may not be able to clearly identify areas of the artery vessel wall where there is significant buildup of bad plaque. Such areas of buildup of bad plaque within an artery vessel wall can be indicators of a patient at high risk of suffering a cardiovascular event, such as a heart attack. In certain circumstances, areas where there exist areas of bad plaque can lead to a rupture wherein there is a release of the fatty materials into the bloodstream of the artery, which in turn can cause a clot to develop in the artery. A blood clot in the artery can cause a stoppage of blood flow to the heart tissue, which can result in a heart attack. Accordingly, there is a need for new technology for analyzing artery vessel walls and/or identifying areas within artery vessel walls that comprise a buildup of plaque whether it be bad or otherwise.

In some embodiments, the systems, devices, and methods described herein are configured to utilize non-invasive medical imaging technologies, such as a CT image or CCTA for example, which can be inputted into a computer system configured to automatically and/or dynamically analyze the medical image to identify one or more coronary arteries and/or plaque within the same. For example, in some embodiments, the system can be configured to utilize one or more machine learning and/or artificial intelligence algorithms to automatically and/or dynamically analyze a medical image to identify, quantify, and/or classify one or more coronary arteries and/or plaque. In some embodiments, the system can be further configured to utilize the identified, quantified, and/or classified one or more coronary arteries and/or plaque to generate a treatment plan, track disease progression, and/or generate a patient-specific medical report, for example using one or more artificial intelligence and/or machine learning algorithms. In some embodiments, the system can be further configured to dynamically and/or automatically generate a visualization of the identified, quantified, and/or classified one or more coronary arteries and/or plaque, for example in the form of a graphical user interface. Further, in some embodiments, to calibrate medical images obtained from different medical imaging scanners and/or different scan parameters or environments, the system can be configured to utilize a normalization device comprising one or more compartments of one or more materials.

As will be discussed in further detail, the systems, devices, and methods described herein allow for automatic and/or dynamic quantified analysis of various parameters relating to plaque, cardiovascular arteries, and/or other structures. M ore specifically, in some embodiments described herein, a medical image of a patient, such as a coronary CT image or CCTA, can be taken at a medical facility. Rather than having a physician eyeball or make a general assessment of the patient, the medical image is transmitted to a backend main server in some embodiments that is configured to conduct one or more analyses thereof in a reproducible manner. As such, in some embodiments, the systems, methods, and devices described herein can provide a quantified measurement of one or more features of a coronary CT image using automated and/or dynamic processes. For example, in some embodiments, the main server system can be configured to identify one or more vessels, plaque, fat, and/or one or more measurements thereof from a medical image. Based on the identified features, in some embodiments, the system can be configured to generate one or more quantified measurements from a raw medical image, such as radiodensity of one or more regions of plaque, identification of stable plaque and/or unstable plaque, volumes thereof, surface areas thereof, geometric shapes, heterogeneity thereof, and/or the like. In some embodiments, the system can also generate one or more quantified measurements of vessels from the raw medical image, such as for example diameter, volume, morphology, and/or the like. Based on the identified features and/or quantified measurements, in some embodiments, the system can be configured to generate a risk and/or disease state assessment and/or track the progression of a plaque-based disease or condition, such as for example atherosclerosis, stenosis, and/or ischemia, using raw medical images. Further, in some embodiments, the system can be configured to generate a visualization or GUI of one or more identified features and/or quantified measurements, such as a quantized color mapping of different features. In some embodiments, the systems, devices, and methods described herein are configured to utilize medical image-based processing to assess for a subject his or her risk of a cardiovascular event, major adverse cardiovascular event (MACE), rapid plaque progression, and/or non-response to medication. In particular, in some embodiments, the system can be configured to automatically and/or dynamically assess such health risk of a subject by analyzing only non-invasively obtained medical images. In some embodiments, one or more of the processes can be automated using an artificial intelligence (AI) and/or machine learning (ML) algorithm. In some embodiments, one or more of the processes described herein can be performed within minutes in a reproducible manner. This is in stark contrast to existing measures today which do not produce reproducible prognosis or assessment, take extensive amounts of time, and/or require invasive procedures. In some embodiments, the systems, methods, and devices described herein comprise and/or are configured to utilize any one or more of such techniques described in US Patent Application Publication No. US 2021/0319558, which is incorporated herein by reference in its entirety.

As such, in some embodiments, the systems, devices, and methods described herein are able to provide physicians and/or patients specific quantified and/or measured data relating to a patient's plaque and/or ischemia that do not exist today. In some embodiments, such detailed level of quantified plaque parameters from image processing and downstream analytical results can provide more accurate and useful tools for assessing the health and/or risk of patients in completely novel ways.

Disclosed are methods for identification of high-risk plaques using volumetric characterization of coronary plaque and perivascular adipose tissue data by computed tomography (CT) scanning. The volumetric characterization of the coronary plaque and perivascular adipose tissue allows for determination of the inflammatory status of the plaque by CT scanning. This is of use in the diagnosis, prognosis, and treatment of coronary artery disease. While certain example embodiments are shown by way of example in the drawings and will herein be described in detail, these embodiments are capable of various modifications and alternative forms. There is no intent to limit example embodiments to the particular forms disclosed, but on the contrary, example embodiments are to cover all modifications, equivalents, and alternatives falling within the scope of example embodiments.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments. As used herein, the term “and/or” includes all combinations of one or more of the associated listed items.

The terminology used herein is for the purpose of describing embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including” when used herein, specify the presence of stated features, integers, steps, operations, elements, and/or components, but do not preclude the presence or addition of one or more other features, integers, steps, operations, elements, components, and/or groups thereof. In this specification, the term “and/or” picks out each individual item as well as all combinations of them.

Unless otherwise defined, all terms (including technical and scientific terms) used herein have the same meaning as commonly understood by one of ordinary skill in the art to which example embodiments belongs. It will be further understood that terms, such as those defined in commonly used dictionaries, should be interpreted as having a meaning that is consistent with their meaning in the context of the relevant art and should not be interpreted in an idealized or overly formal sense unless expressly so defined herein. It should also be noted that in some alternative implementations, the functions/acts noted may occur out of the order noted in the figures. For example, two figures shown in succession may in fact be executed concurrently or may sometimes be executed in the reverse order, depending upon the functionality/acts involved.

In the drawings, the dimensions of layers and regions are exaggerated for clarity of illustration. It will also be understood that when a layer (or tissue) is referred to as being “on” another layer or tissue, it can be directly on the other layer or substrate, or intervening layers may also be present. Further, it will be understood that when a layer is referred to as being “under” another layer, it can be directly under, and one or more intervening layers may also be present. In addition, it will also be understood that when a layer is referred to as being ‘between’ two layers, it can be the only layer between the two layers, or one or more intervening layers may also be present. Like reference numerals refer to like elements throughout.

### Overview of Example Processing System to Characterize Coronary Plaque

This disclosure includes methods and systems of using data generated from images collected by scanning a patient's arteries to identify coronary artery plaques that are at higher risk of causing future heart attack or acute coronary syndrome. In particular, the characteristics of perivascular coronary fat, coronary plaque, and/or the coronary lumen, and the relationship of the characteristics of perivascular coronary fat, coronary plaque, and/or the coronary lumen are discussed to determine ways for identifying the coronary plaque that is more susceptible to implication in future ACS, heart attack and death. The images used to generate the image data may be CT images, CCTA images, or images generated using any applicable technology that can depict the relative densities of the coronary plaque, perivascular fat, and coronary lumen. For example, CCTA images may be used to generate two-dimensional (2D) or volumetric (three-dimensional (3-D)) image data, and this image data may be analyzed to determine certain characteristics that are associated with the radiodensities of the coronary plaque, perivascular fat, and/or coronary lumen. In some implementations, the Hounsfield scale is used to provide a measure of the radiodensity of these features. A Hounsfield unit, as is known, represents an arbitrary unit of x-ray attenuation used for CT scans. Each pixel (2D) or voxel (3D) of a feature in the image data may be assigned a radiodensity value on the Hounsfield scale, and then these values characterizing the features may be analyzed.

In various embodiments, processing of image information may include: (1) determining scan parameters (for example, mA (milliampere), kVp (peak kilovoltage)); (2) determining the scan image quality (e.g., noise, signal-to-noise ratio, contrast to noise ratio); (3) measuring scan-specific coronary artery lumen densities (e.g., from a point distal to a coronary artery wall to a point proximal to the coronary artery wall to distal to the coronary artery, and from a central location of the coronary artery to an outer location (e.g., outer relative to radial distance from the coronary artery): (4) measuring scan-specific plaque densities (e.g., from central to outer, abruptness of change within a plaque from high-to-low or low-to-high) as a function of their 3D shape; and (5) measuring scan-specific perivascular coronary fat densities (from close to the artery to far from the artery) as a function of its 3D shape.

From these measurements, which are agnostic to any commonly known features of ischemia-causing atherosclerosis, the systems and methods of some embodiments described herein can determine several characteristics, including but not limited to one or more of:
1. A ratio of lumen attenuation to plaque attenuation, wherein the volumetric model of scan-specific attenuation density gradients within the lumen adjusts for reduced luminal density across plaque lesions that are more functionally significant in terms of risk value;
2. A ratio of plaque attenuation to fat attenuation, wherein plaques with high radiodensities are considered to present a lower risk, even within a subset of plaques considered “calcified,” where there can be a gradation of densities (for example, 130 to 4000 HU) and risk is considered to be reduced as density increases;
3. A ratio of lumen attenuation/plaque attenuation/fat attenuation;
4. A ratio of #1-3 as a function of 3D shape of atherosclerosis, which can include a 3D texture analysis of the plaque;
5. The 3D volumetric shape and path of the lumen along with its attenuation density from the beginning to the end of the lumen;
6. The totality of plaque and plaque types before and after any given plaque to further inform its risk; or
7. Determination of “higher plaque risks” by “subtracting” calcified (high-density) plaques to obtain a better absolute measure of high risk plaques (lower-density plaques). In other words, this particular embodiment involves identifying calcified plaque and excluding it from further analysis of plaque for the purpose of identifying high risk plaques.

### Other Characteristics can Also be Determined.

The above listed characteristics/metrics, and others, can be analyzed together to assess the risk of the plaque being implicated in future heart attack, ACS, ischemia, or death. This can be done through development and/or validation of a traditional risk score or through machine learning methods. Factors for analysis from the metrics that are likely to be associated with heart attack, ACS, ischemia or death, may include one or more of: (1) a ratio of [bright lumen: dark plaque]; (2) a ratio of [dark plaque: light fat]; (3) a ratio of [bright lumen: dark plaque: light fat]; or (4) a low ratio of [dark lumen: dark myocardium in 1 vessel area]/[lumen: myocardium in another vessel area]. Some improvements in the disclosed methods and systems may include one or more of: (1) using numerical values from ratios of [lumen:plaque], [plaque:fat] and [lumen:plaque: fat] instead of using qualitative definitions of atherosclerotic features; (2) using a scan-specific [lumen:plaque attenuation] ratio to characterize plaque; (3) using a scan-specific [plaque: fat attenuation] ratio to characterize plaque; (4) using ratios of [lumen:plaque: fat circumferential] to characterize plaque; or (5) integration of plaque volume and type before and after as a contributor to risk for any given individual plaque.

A therosclerotic plaque features may change over time with medical treatment (e.g., colchicine and statin medications) and while some of these medications may retard progression of plaque, they also have very important roles in promoting the change in plaque. While statin medications may have reduced the overall progression of plaque, they may also have actually resulted in an increased progression of calcified plaque and a reduction of non-calcified plaque. This change will be associated with a reduction in heart attack or ACS or death, and the disclosed methods can be used to monitor the effects of medical therapy on plaque risk over time. Also, this method can also be used to identify individuals whose atherosclerotic plaque features or [lumen: plaque]/[plaque:fat]/[lumen:plaque:fat] ratios indicate that they are susceptible to rapid progression or malignant transformation of disease. In addition, these methods can be applied to single plaques or to a patient-basis wherein whole-heart atherosclerosis tracking can be used to monitor risk to the patient for experiencing heart attack (rather than trying to identify any specific plaque as being causal for future heart attack). Tracking can be done by automated co-registration processes of image data associated with a patient over a period of time.

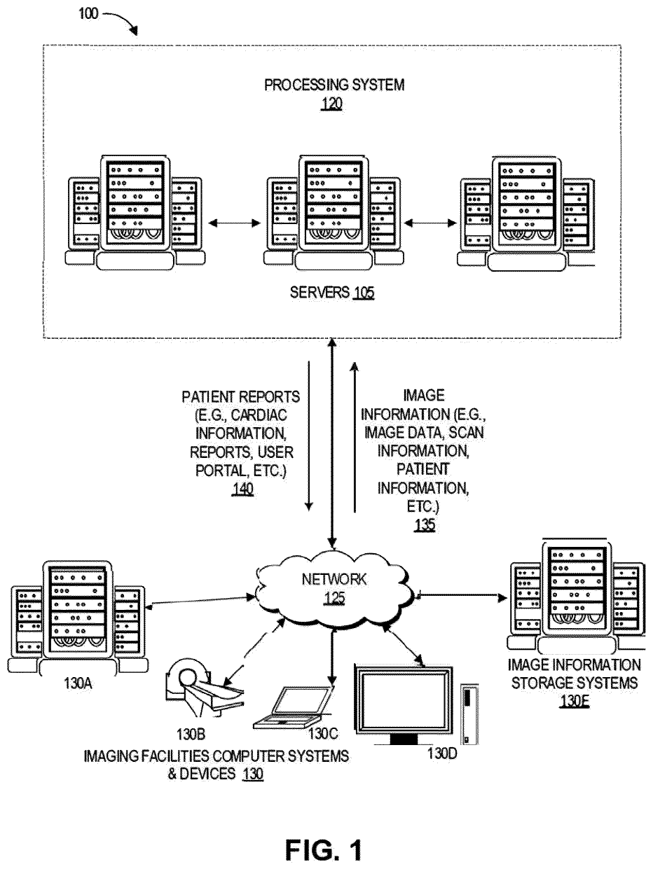

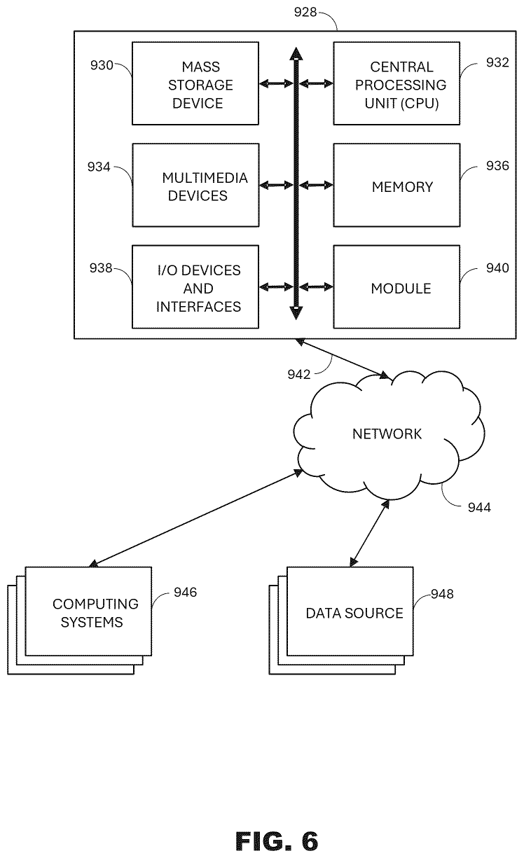

FIG. 1 depicts a schematic of an example of an embodiment of a system 100 that includes a processing system 120 configured to characterize coronary plaque. The processing system 120 may include one or more servers (or computers) 105 each configured with one or more processors. In some embodiments, the processing system 120 includes non-transitory computer memory components for storing data and non-transitory computer memory components for storing instructions that are executed by the one or more processors, the instructions causing the one or more processors to perform methods of analyzing image information. A more detailed example of a server/computer 105 is described in reference to FIG. 6.

In some embodiments, the system 100 also includes a network. The processing system 120 can be in communication with the network 125. The network 125 may include, as at least a portion of the network 125, the Internet, a wide area network (WAN), a wireless network, or the like. In some embodiments, the processing system 120 is part of a “cloud” implementation, which can be located anywhere that is in communication with the network 125. In some embodiments, the processing system 120 is located in the same geographic proximity as an imaging facility that images and stores patient image data. In some embodiments, the processing system 120 is located remotely from where the patient image data is generated or stored.

FIG. 1 also illustrates in system 100 various computer systems and devices 130 (e.g., of an imaging facility) that may be related to generating patient image data and that are also connected to the network 125. One or more of the devices 130 may be at an imaging facility that generates images of a patient's arteries, a medical facility (e.g., a hospital, doctor's office, etc.) or may be the personal computing device of a patient or care provider. For example, as illustrated in FIG. 1, an imaging facility server (or computer) 130A may be connected to the network 125. Also, in this example, a scanner 130B in an imaging facility may be connected to the network 125. One or more other computer devices may also be connected to the network 125. For example, a laptop 130C, a personal computer 130D, and/or and an image information storage system 130E may also be connected to the network 125, and communicate with the processing system 120, and each other, via the network 125.

In some examples, the scanner 130B can be a computed tomography (CT) scanner that uses a rotating X-ray tube and a row of detectors to measure X-ray attenuations by different tissues in the body and form a corresponding image. In another example, a scanner 130B can use a spinning tube (“spiral CT”) in which an entire X-ray tube and detectors are spun around a central axis of the area being scanned. In another example, the scanner 130B can utilize electron beam tomography (EBT). In another example, the scanner 130B can be a dual source CT scanner with two X-ray tube systems. In another example, the scanner 130B can be a multi-source CT scanner with more than two X-ray tube systems. In another example, the scanner 130B can include a fast switching X-ray tube system. The methods and systems described herein can also use images from other CT scanners. In some examples, the scanner 130B is a photon counting CT scanner, a spectral CT scanner, or a dual energy CT scanner. A photon counting CT scanner, a spectral CT scanner, or a dual energy CT scanner can help provide more detailed higher resolution images that better show small blood vessels, plaque, and other vascular pathologies, and allow for the determination of absolute material densities over relative densities. In general, a photon counting CT scanner may use an X-ray detector to count photons and quantifies the energy, determining the count of the number of photons in several discrete energy bins, resulting in higher contrast to noise ratio, and improved spatial resolution and spectral imaging compared to conventional CT scanners. Each registered photon can be assigned to a specific bin depending on its energy, such that each pixel measures a histogram of the incident X-ray spectrum. This spectral information can provide several advantages. First, it can be used to quantitatively determine the material composition of each pixel in the reconstructed CT image, as opposed to the estimated average linear attenuation coefficient obtained in a conventional CT scan. The spectral/energy information can be used to remove beam hardening artifacts that occur higher linear attenuation of many materials that shifts mean energy of the X-ray spectrum towards higher energies. Also, use of more than two energy bins can allow discrimination between objects (bone, calcifications, contrast agents, tissue, etc.). In some embodiments, images generated using a photon counting CT scanner can allow assessment of plaques at different monochromatic energies as well as different polychromatic spectra (e.g., 100 KV p, 120 kV p, 140 kV p, etc.), and this can change definition of non-calcified and calcified plaques compared to conventional CT scanners. A spectral CT scanner can use different X-ray wavelengths (or energies) to produce a CT scan. A dual energy CT scanner can use separate X-ray energies to detect two different energy ranges. In an example, a dual energy CT scanner (also known as spectral CT) can use an X-ray detector with separate layers to detect two different energy ranges (′dual layer′). In another example, a dual energy CT scanner can use a single scanner to scan twice using two different energy levels (e.g., electronic kVp switching). Images can be formed from combining the images detected at each different energy level, or the images may be used separately to assess a medical condition of a patient. In addition to providing absolute material densities, a photon counting CT scanner can also allow for evaluation of images that are “monochromatic” as opposed to the typical CT, which is polychromatic spectra of light. As noted above, features (e.g., low density non-calcified plaque, calcified plaque, non-calcified plaque) that are depicted images formed using a photon counting CT scanner, a spectral CT scanner, or a dual energy CT scanner may have different radiodensities than those depicted in images formed from a conventional CT scanner, that is, such images may affect or change the definition of calcified and non-calcified plaque. However, radiodensities of calcified and non-calcified plaque, or other features depicted in images formed from a photon counting CT scanner, a spectral CT scanner, or a dual energy CT scanner, can be normalized to correspond to densities of conventional CT scanners and to the densities disclosed herein. Accordingly, the radiodensities disclosed herein can be directly correlated to radiodensities of images generated with a photon counting CT scanner, a spectral CT scanner, or a dual energy CT scanner such that the systems and methods, analysis, plaque densities etc. disclosed herein are directly applicable to images formed from a photon counting CT scanner, a spectral CT scanner, or a dual energy CT scanner, and can be directly applicable to images formed from a photon counting CT scanner, a spectral CT scanner, or a dual energy CT scanner that are normalized to equivalent conventional CT scanner radiodensities.

The information communicated from the devices 130 to the processing system 120 via the network 125 may include image information 135. In various embodiments, the image information 135 may include 2D or 3D image data of a patient, scan information related to the image data, patient information, and other imagery or image related information that relates to a patient. For example, the image information may include patient information including (one or more) characteristics of a patient, for example, age, gender, body mass index (BMI), medication, blood pressure, heart rate, height, weight, race, whether the patient is a smoker or non-smoker, body habitus (for example, the “physique” or “body type” which may be based on a wide range of factors), medical history, diabetes, hypertension, prior coronary artery disease (CAD), dietary habits, drug history, family history of disease, information relating to other previously collected image information, exercise habits, drinking habits, lifestyle information, lab results and the like. In some embodiments, the image information includes identification information of the patient, for example, patient's name, patient's address, driver's license number, Social Security number, or indicia of another patient identification. Once the processing system 120 analyzes the image information 135, information relating to a patient 140 may be communicated from the processing system 120 to a device 130 via the network 125. The patient information 140 may include for example, a patient report. Also, the patient information 140 may include a variety of patient information which is available from a patient portal, which may be accessed by one of the devices 130.

In some embodiments, image information comprising a plurality of images of a patient's coronary arteries and patient information/characteristics may be provided from one or more of the devices 130 to the one or more servers 105 of the processing system 120 via a network 125. In some embodiments, the processing system 120 is configured to generate coronary artery information using the plurality of images of the patient's coronary arteries to generate two-dimensional and/or three-dimensional data representations of the patient's coronary arteries. In some embodiments, the processing system 120 analyzes the data representations to generate patient reports documenting a patient's health conditions and risks related to coronary plaque. The patient reports may include images and graphical depictions of the patient's arteries in the types of coronary plaque in or near the coronary arteries. Using machine learning techniques or other artificial intelligence techniques, the data representations of the patient's coronary arteries may be compared to other patients' data representations (e.g., that are stored in a database) to determine additional information about the patient's health. For example, based on certain plaque conditions of the patient's coronary arteries, the likelihood of a patient having a heart attack or other adverse coronary effect can be determined. Also, for example, additional information about the patient's risk of CAD may also be determined.

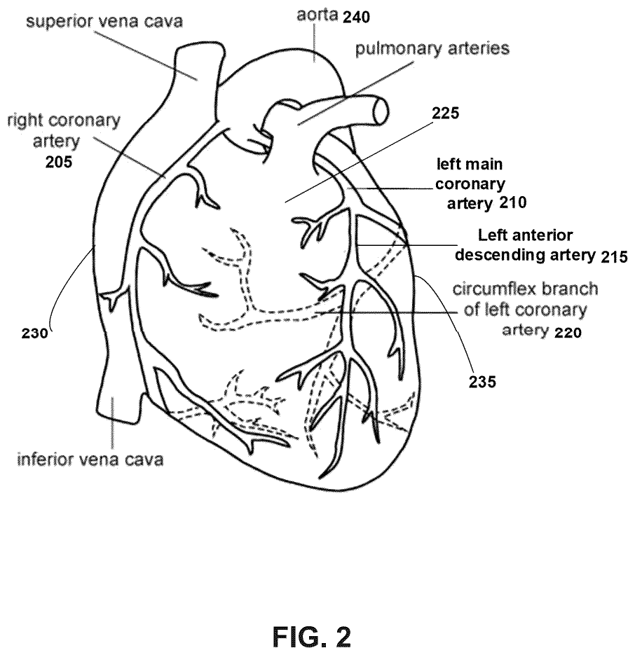

FIG. 2 is a schematic illustrating an example of a heart muscle 225 and its coronary arteries. The coronary vasculature includes a complex network of vessels ranging from large arteries to arterioles, capillaries, venules, veins, etc. FIG. 2 depicts a model of a portion of the coronary vasculature that circulates blood to and within the heart and includes an aorta 240 that supplies blood to a plurality of coronary arteries, for example, a left anterior descending (LAD) artery 215, a left circumflex (LCX) artery 220, and a right coronary (RCA) artery 205, described further below. Coronary arteries supply blood to the heart muscle 225. Like all other tissues in the body, the heart muscle 225 needs oxygen-rich blood to function. Also, oxygen-depleted blood must be carried away. The coronary arteries wrap around the outside of the heart muscle 225. Small branches dive into the heart muscle 225 to bring it blood. The examples of methods and systems described herein may be used to determine information relating to blood flowing through the coronary arteries in any vessels extending therefrom. In particular, the described examples of methods and systems may be used to determine various information relating to one or more portions of a coronary artery where plaque has formed which is then used to determine risks associated with such plaque, for example, whether a plaque formation is a risk to cause an adverse event to a patient.

The right side 230 of the heart 225 is depicted on the left side of FIG. 2 (relative to the page) and the left side 235 of the heart is depicted on the right side of FIG. 2. The coronary arteries include the right coronary artery (RCA) 205 which extends from the aorta 240 downward along the right side 230 of the heart 225, and the left main coronary artery (LMCA) 210 which extends from the aorta 240 downward on the left side 235 of the heart 225. The RCA 205 supplies blood to the right ventricle, the right atrium, and the SA (sinoatrial) and AV (atrioventricular) nodes, which regulate the heart rhythm. The RCA 205 divides into smaller branches, including the right posterior descending artery and the acute marginal artery. Together with the left anterior descending artery 215, the RCA 205 helps supply blood to the middle or septum of the heart.

The LMCA 210 branches into two arteries, the anterior interventricular branch of the left coronary artery, also known as the left anterior descending (LAD) artery 215 and the circumflex branch of the left coronary artery 220. The LAD artery 215 supplies blood to the front of the left side of the heart. Occlusion of the LAD artery 215 is often called the widow-maker infarction. The circumflex branch of the left coronary artery 220 encircles the heart muscle. The circumflex branch of the left coronary artery 220 supplies blood to the outer side and back of the heart, following the left part of the coronary sulcus, running first to the left and then to the right, reaching nearly as far as the posterior longitudinal sulcus.

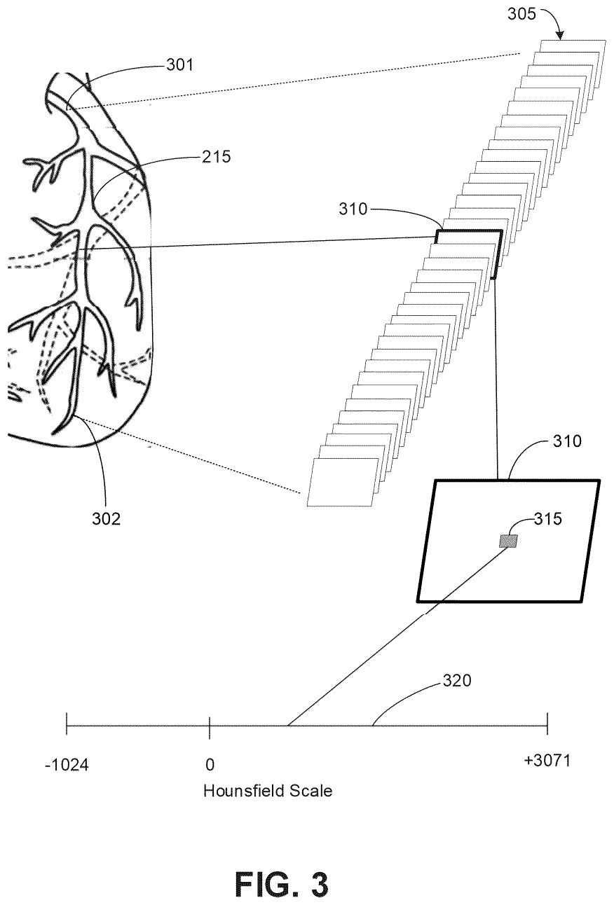

FIG. 3 illustrates an example of a set of images generated from scanning along a coronary artery, including a selected image of a portion of a coronary artery, and how image data may correspond to a value on the Hounsfield Scale. As discussed in reference to FIG. 1, in addition to obtaining image data, scan information including metrics related to the image data, and patient information including characteristics of the patient may also be collected.

A portion of a heart 225, the LMCA 210, and the LAD artery 215 is illustrated in the example of FIG. 3. A set of images 305 can be collected along portions of the LMCA 210 and the LAD artery 215, in this example from a first point 301 on the LMCA 210 to a second point 302 on the LAD artery 215. In some examples, the image data may be obtained using noninvasive imaging methods. For example, CCTA image data can be generated using a scanner to create images of the heart in the coronary arteries and other vessels extending therefrom. Collected CCTA image data may be subsequently used to generate three-dimensional image models of the features contained in the CCTA image data (for example, the right coronary artery 205, the left main coronary artery 210, the left anterior descending artery 215, the circumflex branch of the left coronary artery 220, the aorta 240, and other vessels related to the heart that appear in the image data.

In various embodiments, different imaging methods may be used to collect the image data. For example, ultrasound or magnetic resonance imaging (MRI) may be used. In some embodiments, the imaging methods involve using a contrast agent to help identify structures of the coronary arteries, the contrast agent being injected into the patient prior to the imaging procedure. The various imaging methods may each have their own advantages and disadvantages of usage, including resolution and suitability of imaging the coronary arteries. Imaging methods which may be used to collect image data of the coronary arteries are constantly improving as improvements to the hardware (e.g., sensors and emitters) and software are made. The disclosed systems and methods contemplate using CCTA image data and/or any other type of image data that can provide or be converted into a representative 3D depiction of the coronary arteries, plaque contained within the coronary arteries, and perivascular fat located in proximity to the coronary arteries containing the plaque such that attenuation or radiodensity values of the coronary arteries, plaque, and/or perivascular fat can be obtained. In some embodiments, the imaging modality can comprise one or more of CT, Dual-Energy Computed Tomography (DECT), Spectral CT, photon-counting CT, x-ray, ultrasound, echocardiography, intravascular ultrasound (IVUS), Magnetic Resonance (MR) imaging, optical coherence tomography (OCT), nuclear medicine imaging, positron-emission tomography (PET), single photon emission computed tomography (SPECT), near-field infrared spectroscopy (NIRS), contrast-enhanced CT, or non-contrast CT.

Referring still to FIG. 3, a particular image 310 of the image data 305 is shown, which represents an image of a portion of the left anterior descending artery 215. The image 310 includes image information, the smallest point of the information manipulated by a system referred to herein generally as a pixel, for example pixel 315 of image 310. The resolution of the imaging system used to capture the image data will affect the size of the smallest feature that can be discerned in an image. In addition, subsequent manipulation of the image may affect the dimensions of a pixel. As one example, the image 310 in a digital format, may contain 4000 pixels in each horizontal row, and 3000 pixels in each vertical column. Pixel 315, and each of the pixels in image data 310 and in the image data 305, can be associated with a radiodensity value that corresponds to the density of the pixel in the image. Illustratively shown in FIG. 3 is mapping pixel 315 to a point on the Hounsfield scale 320. The Hounsfield scale 320 is a quantitative scale for describing radiodensity. The Hounsfield unit scale linear transformation of the original linear attenuation coefficient measurement into one in which the radiodensity of distilled water at standard pressure and temperature is defined as zero Hounsfield units (HU), while the radiodensity of air at standard pressure and temperature is defined as −1000 HU. Although FIG. 3 illustrates an example of mapping pixel 315 of image 310 to a point on the Hounsfield scale 320, such an association of a pixel to a radiodensity value can also be done with 3D data. For example, after the image data 305 is used to generate a three-dimensional representation of the coronary arteries.

Once the data has been obtained and rendered into a three-dimensional representation, various processes can be performed on the data to identify areas of analysis. For example, a three-dimensional depiction of a coronary artery may be segmented to define a plurality of portions of the artery and identified as such in the data. In some embodiments, the data may be filtered (e.g., smoothed) by various methods to remove anomalies that are the result of scanning or other various errors. Various known methods for segmenting and smoothing the 3D data may be used, and therefore for brevity of the disclosure will not be discussed in any further detail herein.

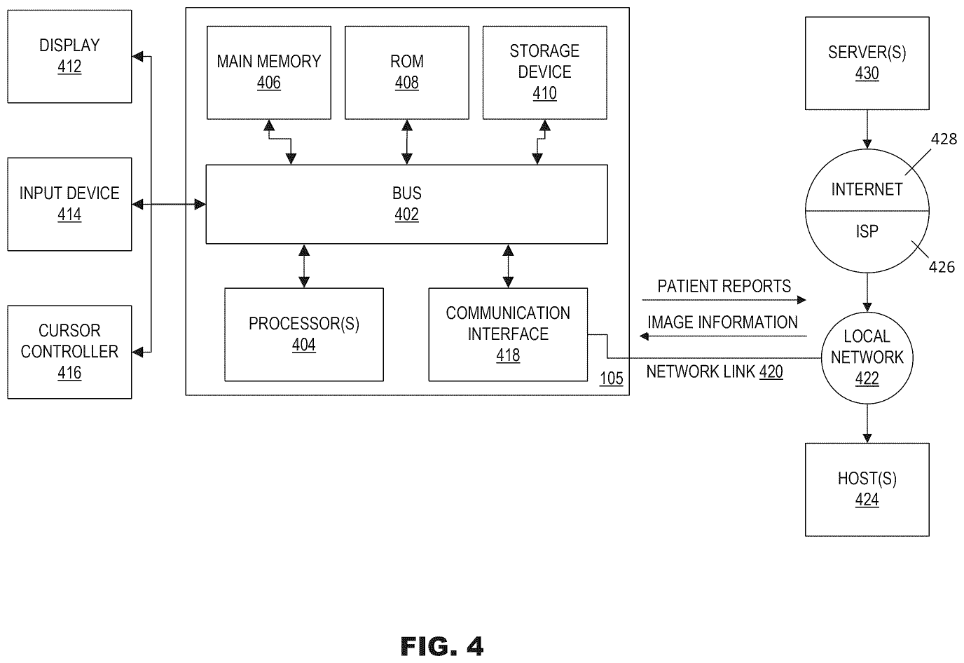

FIG. 4 is a block diagram that illustrates a computer system 400 upon which various embodiments may be implemented. In some embodiments, the computer system 400 includes a bus 402 or other communication mechanism for communicating information, and a hardware processor, or multiple processors, 404 coupled with bus 402 for processing information. Hardware processor(s) 404 may be, for example, one or more general purpose microprocessors.

In some embodiments, the computer system 400 also includes a main memory 406, such as a random access memory (RAM), cache and/or other dynamic storage devices, coupled to bus 402 for storing information and instructions to be executed by processor 404. Main memory 406 also may be used for storing temporary variables or other intermediate information during execution of instructions to be executed by processor 404. Such instructions, when stored in storage media accessible to processor 404, may render the computer system 400 into a special-purpose machine that is customized to perform the operations specified in the instructions. The main memory 406 may, for example, include instructions that analyze image information to determine characteristics of coronary features (e.g., plaque, perivascular fat, and coronary arteries) to produce patient reports containing information that characterizes aspects of the patient's health relating to their coronary arteries. For example, one or more metrics may be determined, the metrics including one or more of a slope/gradient of a feature, a maximum density, minimum density, a ratio of a slope of one feature to the slope of another feature, a ratio of a maximum density of one feature to the maximum density of another feature, a ratio of a minimum density of a feature to the minimum density of the same feature, or a ratio of the minimum density of a feature to the maximum density of another feature.

In some embodiments, the computer system 400 further includes a read only memory (ROM) 408 or other static storage device coupled to bus 402 for storing static information and instructions for processor 404. In some embodiments, a storage device 410, such as a magnetic disk, optical disk, or USB thumb drive (Flash drive), etc., is provided and coupled to bus 402 for storing information and instructions.

Computer system 400 may be coupled via bus 402 to a display 412, such as a cathode ray tube (CRT) or LCD display (or touch screen), for displaying information to a computer user. In some embodiments, an input device 414, including alphanumeric and other keys, is coupled to bus 402 for communicating information and command selections to processor 404. Another type of user input device can include cursor control 416, such as a mouse, a trackball, or cursor direction keys for communicating direction information and command selections to processor 404 and for controlling cursor movement on display 412. In some embodiments, this input device typically has two degrees of freedom in two axes, a first axis (e.g., x) and a second axis (e.g., y), that allows the device to specify positions in a plane. In some embodiments, the same direction information and command selections as cursor control may be implemented via receiving touches on a touch screen without a cursor.

Computing system 400 may include a user interface module to implement a GUI that may be stored in a mass storage device as computer executable program instructions that are executed by the computing device(s). Computer system 400 may further, as described below, implement the techniques described herein using customized hard-wired logic, one or more ASICs or FPGAS, firmware, and/or program logic which in combination with the computer system causes or programs computer system 400 to be a special-purpose machine. According to some embodiments, the techniques herein are performed by computer system 400 in response to processor(s) 404 executing one or more sequences of one or more computer readable program instructions contained in main memory 406. Such instructions may be read into main memory 406 from another storage medium, such as storage device 410. In some embodiments, execution of the sequences of instructions contained in main memory 406 causes processor(s) 404 to perform the process steps described herein. In some embodiments, hard-wired circuitry may be used in place of or in combination with software instructions.

Various forms of computer readable storage media may be involved in carrying one or more sequences of one or more computer readable program instructions to processor 404 for execution. For example, the instructions may initially be carried on a magnetic disk or solid state drive of a remote computer. The remote computer can load the instructions into its dynamic memory and send the instructions over a telephone line using a modem. A modem local to computer system 400 can receive the data on the telephone line and use an infra-red transmitter to convert the data to an infra-red signal. An infra-red detector can receive the data carried in the infra-red signal and appropriate circuitry can place the data on bus 402. Bus 402 carries the data to main memory 406, from which processor 404 retrieves and executes the instructions. The instructions received by main memory 406 may optionally be stored on storage device 410 either before or after execution by processor 404.

In some embodiments, the computer system 400 also includes a communication interface 418 coupled to bus 402. In some embodiments, the communication interface 418 provides a two-way data communication coupling to a network link 420 that is connected to a local network 422. For example, communication interface 418 may be an integrated services digital network (ISDN) card, cable modem, satellite modem, or a modem to provide a data communication connection to a corresponding type of telephone line. As another example, communication interface 418 may be a local area network (LAN) card to provide a data communication connection to a compatible LA N (or WAN component to communicate with a WAN). Wireless links may also be implemented. In any such implementation, communication interface 418 sends and receives electrical, electromagnetic, or optical signals that carry digital data streams representing various types of information.

In some embodiments, the network link 420 typically provides data communication through one or more networks to other data devices. For example, network link 420 may provide a connection through local network 422 to a host computer 424 or to data equipment operated by an Internet Service Provider (ISP) 426. In some embodiments, the ISP 426 in turn provides data communication services through the worldwide packet data communication network now commonly referred to as the “Internet” 428. Local network 422 and Internet 428 may both use electrical, electromagnetic, or optical signals that carry digital data streams. The signals through the various networks and the signals on network link 420 and through communication interface 418, which carry the digital data to and from computer system 400, are example forms of transmission media.

Computer system 400 can send messages and receive data, including program code, through the network(s), network link 420 and communication interface 418. In the Internet example, a server 430 might transmit a requested code for an application program through Internet 428, ISP 426, local network 422 and communication interface 418.

The received code may be executed by processor 404 as it is received, and/or stored in storage device 410, or other non-volatile storage for later execution.

Accordingly, in some embodiments, the computer system 105 comprises a non-transitory computer storage medium storage device 410 configured to at least store image information of patients. The computer system 105 can also include non-transitory computer storage medium storage that stores instructions for the one or more processors 404 to execute a process (e.g., a method) for characterization of coronary plaque tissue data and perivascular tissue data using image data gathered from a computed tomography (CT) scan along a blood vessel, the image information including radiodensity values of coronary plaque and perivascular tissue located adjacent to the coronary plaque. Executing the instructions, in some embodiments, the one or more processors 404 can quantify, in the image data, the radiodensity in regions of coronary plaque, quantify in the image data, radiodensity in at least one region of corresponding perivascular tissue adjacent to the coronary plaque, determine gradients of the quantified radiodensity values within the coronary plaque and the quantified radiodensity values within the corresponding perivascular tissue, determine a ratio of the quantified radiodensity values within the coronary plaque and the corresponding perivascular tissue, and characterizing the coronary plaque by analyzing one or more of the gradients of the quantified radiodensity values in the coronary plaque and the corresponding perivascular tissue, or the ratio of the coronary plaque radiodensity values and the radiodensity values of the corresponding perivascular tissue.

Various embodiments of the present disclosure may be a system, a method, and/or a computer program product at any possible technical detail level of integration. The computer program product may include a computer readable storage medium (or mediums) having computer readable program instructions thereon for causing a processor to carry out aspects of the present disclosure. For example, the functionality described herein may be performed as software instructions are executed by, and/or in response to software instructions being executed by, one or more hardware processors and/or any other suitable computing devices. The software instructions and/or other executable code may be read from a computer readable storage medium (or mediums).

The computer readable storage medium can be a tangible device that can retain and store data and/or instructions for use by an instruction execution device. The computer readable storage medium may be, for example, but is not limited to, an electronic storage device (including any volatile and/or non-volatile electronic storage devices), a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a solid state drive, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. In some embodiments, a network adapter card or network interface in each computing/processing device receives computer readable program instructions from the network and forwards the computer readable program instructions for storage in a computer readable storage medium within the respective computing/processing device.

Computer readable program instructions (as also referred to herein as, for example, “code,” “instructions,” “module,” “application,” “software application,” and/or the like) for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. Computer readable program instructions may be callable from other instructions or from itself, and/or may be invoked in response to detected events or interrupts. Computer readable program instructions configured for execution on computing devices may be provided on a computer readable storage medium, and/or as a digital download (and may be originally stored in a compressed or installable format that requires installation, decompression, or decryption prior to execution) that may then be stored on a computer readable storage medium. Such computer readable program instructions may be stored, partially or fully, on a memory device (e.g., a computer readable storage medium) of the executing computing device, for execution by the computing device. The computer readable program instructions may execute entirely on a user's computer (e.g., the executing computing device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Some aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart(s) and/or block diagram(s) block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. For example, the instructions may initially be carried on a magnetic disk or solid state drive of a remote computer. The remote computer may load the instructions and/or modules into its dynamic memory and send the instructions over a telephone, cable, or optical line using a modem. A modem local to a server computing system may receive the data on the telephone/cable/optical line and use a converter device including the appropriate circuitry to place the data on a bus. The bus may carry the data to a memory, from which a processor may retrieve and execute the instructions. The instructions received by the memory may optionally be stored on a storage device (e.g., a solid state drive) either before or after execution by the computer processor.

The flowchart and block diagrams in the figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. In addition, certain blocks may be omitted in some implementations. The methods and processes described herein are also not limited to any particular sequence, and the blocks or states relating thereto can be performed in other sequences that are appropriate.

Based on the identified features and/or quantified measurements, for example from analyzing one or more medical images, in some embodiments, the system can be configured to generate a risk assessment and/or track the progression of a plaque-based disease or condition, such as for example atherosclerosis, stenosis, ischemia, myocardial infarction, and/or major adverse cardiovascular event (MACE), using raw medical images. As described further herein, in some embodiments the system can perform risk assessment and/or tracking the progression of a plaque-based disease based on other patients' information. For example, by comparing or evaluating features in a patient's medical images and patient information (e.g., age, gender, BMI, medication, blood pressure, heart rate, height, weight, race, whether the patient is a smoker or non-smoker, medical history, family history of disease, etc.) to features in other patients' medical images and their associated patient information including their outcome after a period of time.

Further, in some embodiments, the system can be configured to generate a visualization of GUI of one or more identified features and/or quantified measurements, such as a quantized color mapping of different features. In some embodiments, the systems, devices, and methods described herein are configured to utilize medical image-based processing to assess for a subject his or her risk of a cardiovascular event, major adverse cardiovascular event (MACE), rapid plaque progression, and/or response to non-response to medication and/or lifestyle change and/or other treatment and/or invasive procedure. In particular, in some embodiments, the system can be configured to automatically and/or dynamically assess such health risk of a subject by analyzing only non-invasively obtained medical images. In some embodiments, one or more of the processes can be automated using an artificial intelligence (AI) and/or machine learning (ML) algorithm. In some embodiments, one or more of the processes described herein can be performed within minutes in a reproducible manner. This is in stark contrast to existing measures today which do not produce reproducible prognosis or assessment, take extensive amounts of time, and/or require invasive procedures.

In some embodiments, image information comprising a plurality of images of a patient's coronary arteries and patient information/characteristics may be provided from one or more of the devices to the one or more servers of the processing system via a network. The processing system is configured to generate coronary artery information using the plurality of images of the patient's coronary arteries to generate two-dimensional and/or three-dimensional data representations of the patient's coronary arteries. Then, the processing system analyzes the data representations to generate patient reports documenting a patient's health conditions and risks related to coronary plaque. The patient reports may include images and graphical depictions of the patient's arteries in the types of coronary plaque in or near the coronary arteries. Using machine learning techniques or other artificial intelligent techniques, the data representations of the patient's coronary arteries may be compared to other patients' data representations (e.g., that are stored in a database) to determine additional information about the patient's health. In some embodiments, the artificial intelligence can be trained using a dataset of other patients' data representations to identify correlations in data. For example, based on certain plaque conditions of the patient's coronary arteries, the likelihood of a patient having a heart attack or other adverse coronary effect can be determined. Also, for example, additional information about the patient's risk of CAD may also be determined.

In some embodiments, the coronary plaque information of a patient being examined may be compared to or analyzed in reference to a patient who has one or more of the same or similar patient characteristics. For example, the patient being examined may be compared to a patient that has the same or similar characteristics of sex, age, BMI, medication, blood pressure, heart rate, weight, height, race, body habitus, smoking, diabetes, hypertension, prior coronary artery disease, family history, and lab results. Such comparisons can be done through various means, for example machine learning and/or artificial intelligence techniques. In some examples, neural network is used to compare a patient's coronary artery information to numerous (e.g., 10,000+) other patients' coronary artery information. For such patients that have similar patient information and similar cardiac information, risk assessments of the plaque of the patient being examined may be determined.

In some embodiments, Deep Learning (DL) methods, machine learning (ML) methods, and/or artificial intelligence (AI) methods can be used to analyze image information. In an example, this analysis can comprise image segmentation, feature extraction, and classification. In some embodiments, M L methods can comprise image feature extraction and image-based learning from raw data. In some embodiments, the M L method can receive an input of a large training set to learn to ignore variations that could otherwise skew the results of the method. In some embodiments, DL can comprise a Neural Network (NN) with three or more layers that can improve the accuracy of determinations. Advantageously, in some embodiments, DL can obviate the need for pre-processing data and, instead, process raw data. For example, while a human may input a hierarchy of important features of coronary image information for a ML algorithm to make determinations, DL algorithms can determine which features are important and use these features to make determinations. Advantageously, in some embodiments, a DL algorithm can adjust itself for accuracy and precision. In some embodiments, ML and DL algorithms can perform supervised learning, unsupervised learning, and reinforcement learning.

In some embodiments, NN approaches, including convolutional neural networks (CNN) and recurrent convolutional neural networks (RCNN), among others, can be used to analyze information in a manner similar to high-level cognitive functions of a human mind. In some embodiments, a NN approach can comprise training an object recognition system numerous medical images in order to teach it patterns in the images that correlate with particular labels. In some embodiments, a CNN can comprise a NN where the nodes of each layer are clustered, the clusters overlap, and each cluster feeds data to multiple nodes of the next layer. In some embodiments, a RCNN can comprise a CNN where recurrent connections are incorporated in each convolutional layer. Advantageously, in some embodiments, the recurrent connections can make object recognition a dynamic process despite the fact that the input is static.

In some embodiments, the vessel identification algorithm, coronary artery identification algorithm, and/or plaque identification algorithm can be trained on a plurality of medical images wherein one or more vessels, coronary arteries, and/or regions of plaque are pre-identified. Based on such training, for example by use of a CNN in some embodiments, the system can be configured to automatically and/or dynamically identify from raw medical images the presence and/or parameters of vessels, coronary arteries, and/or plaque. In some embodiments, the system can be configured to utilize one or more AI and/or ML algorithms to identify and/or analyze vessels or plaque, derive one or more quantification metrics and/or classifications, and/or generate a treatment plan. In some embodiments, the system can be configured to utilize an AI and/or ML algorithm to identify areas in an artery that exhibit plaque buildup within, along, inside and/or outside the arteries. In some embodiments, input to the AI and/or ML algorithms can include images of a patient and patient information (or characteristics), for example, one or more of age, gender, body mass index (BMI), medication, blood pressure, heart rate, height, weight, race, whether the patient is a smoker or non-smoker, body habitus (for example, the “physique” or “body type” which may be based on a wide range of factors), medical history, diabetes, hypertension, prior coronary artery disease (CAD), dietary habits, drug history, family history of disease, information relating to other previously collected image information, exercise habits, drinking habits, lifestyle information, or lab results, and the like. In an example where a NN is used, the NN can be trained using information from a plurality of patients, where the information for each patient can include medical images and one or more patient characteristics.

In some embodiments, the system can be configured to utilize one or more AI and/or ML algorithms to automatically and/or dynamically identify one or more regions of plaque using image processing. For example, in some embodiments, the one or more AI and/or ML algorithms can be trained using a CNN on a set of medical images on which regions of plaque have been identified, thereby allowing the AI and/or M L algorithm automatically identify regions of plaque directly from a medical image. In some embodiments, the system can be configured to identify a vessel wall and a lumen wall for each of the identified coronary arteries in the medical image. In some embodiments, the system is then configured to determine the volume in between the vessel wall and the lumen wall as plaque. In some embodiments, the system can be configured to identify regions of plaque based on the radiodensity values typically associated with plaque, for example by setting a predetermined threshold or range of radiodensity values that are typically associated with plaque with or without normalizing using a normalization device.

In some embodiments, the one or more vascular morphology parameters and/or plaque parameters can comprise quantified parameters derived from the medical image. For example, in some embodiments, the system can be configured to utilize an AI and/or ML algorithm or other algorithm to determine one or more vascular morphology parameters and/or plaque parameters. A s another example, in some embodiments, the system can be configured to determine one or more vascular morphology parameters, such as classification of arterial remodeling due to plaque, which can further include positive arterial remodeling, negative arterial remodeling, and/or intermediate arterial remodeling. In some embodiments, the classification of arterial remodeling is determined based on a ratio of the largest vessel diameter at a region of plaque to a normal reference vessel diameter of the same region which can be retrieved from a normal database. In some embodiments, the system can be configured to classify arterial remodeling as positive when the ratio of the largest vessel diameter at a region of plaque to a normal reference vessel diameter of the same region is more than 1.1. In some embodiments, the system can be configured to classify arterial remodeling as negative when the ratio of the largest vessel diameter at a region of plaque to a normal reference vessel diameter is less than 0.95. In some embodiments, the system can be configured to classify arterial remodeling as intermediate when the ratio of the largest vessel diameter at a region of plaque to a normal reference vessel diameter is between 0.95 and 1.1.

In some embodiments, the system is configured to classify atherosclerosis of a subject based on the quantified atherosclerosis as one or more of high risk, medium risk, or low risk. In some embodiments, the system is configured to classify atherosclerosis of a subject based on the quantified atherosclerosis using an AI, ML, and/or other algorithm. In some embodiments, the system is configured to classify atherosclerosis of a subject by combining and/or weighting one or more of a ratio of volume of surface area, volume, heterogeneity index, and radiodensity of the one or more regions of plaque.

In some embodiments, the system can be configured to identify one or more regions of fat, such as epicardial fat, in the medical image, for example using one or more AI and/or ML algorithms to automatically and/or dynamically identify one or more regions of fat. In some embodiments, the one or more AI and/or ML algorithms can be trained using a CNN on a set of medical images on which regions of fat have been identified, thereby allowing the AI and/or ML algorithm automatically identify regions of fat directly from a medical image. In some embodiments, the system can be configured to identify regions of fat based on the radiodensity values typically associated with fat, for example by setting a predetermined threshold or range of radiodensity values that are typically associated with fat with or without normalizing using a normalization device.

In some embodiments, the system is configured to utilize an AI, ML, and/or other algorithm to characterize the change in calcium score based on one or more plaque parameters derived from a medical image. For example, in some embodiments, the system can be configured to utilize an AI and/or ML algorithm that is trained using a CNN and/or using a dataset of known medical images with identified plaque parameters combined with calcium scores. In some embodiments, the system can be configured to characterize a change in calcium score by accessing known datasets of the same stored in a database. For example, the known dataset may include datasets of changes in calcium scores and/or medical images and/or plaque parameters derived therefrom of other subjects in the past. In some embodiments, the system can be configured to characterize a change in calcium score and/or determine a cause thereof on a vessel-by-vessel basis, segment-by-segment basis, plaque-by-plaque basis, and/or a subject basis.

In some embodiments, the systems disclosed herein can be used to dynamically and automatically determine a necessary stent type, length, diameter, gauge, strength, and/or any other stent parameter for a particular patient based on processing of the medical image data, for example using AI, ML, and/or other algorithms.

In some embodiments, the system can be configured to utilize an AI and/or M L algorithm to generate the patient-specific report. In some embodiments, the patient-specific report can include a document, AR experience, VR experience, video, and/or audio component.

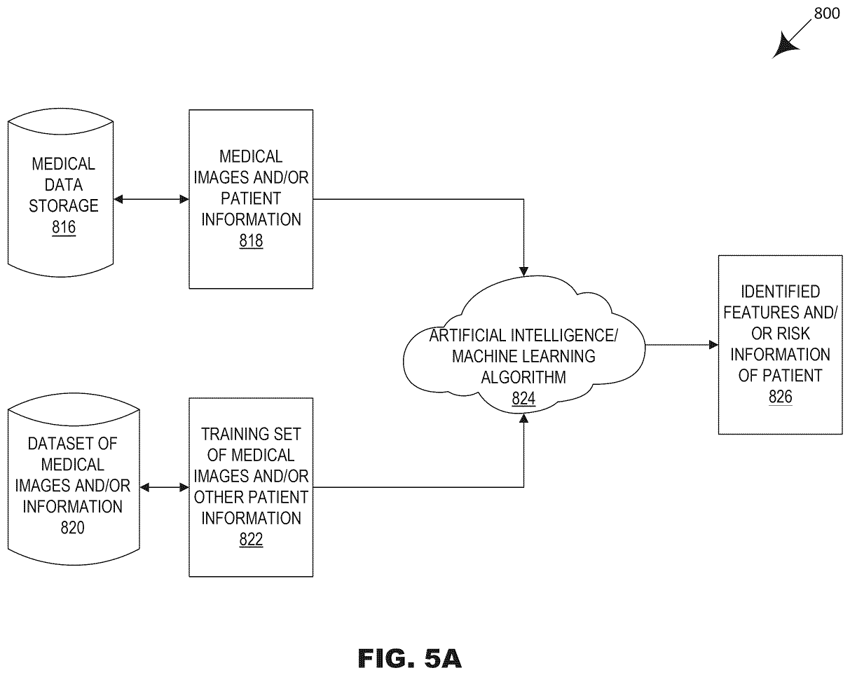

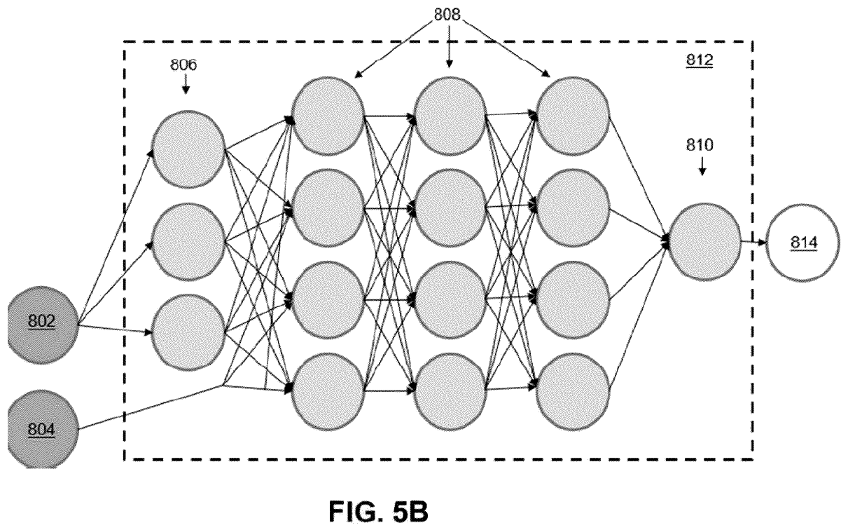

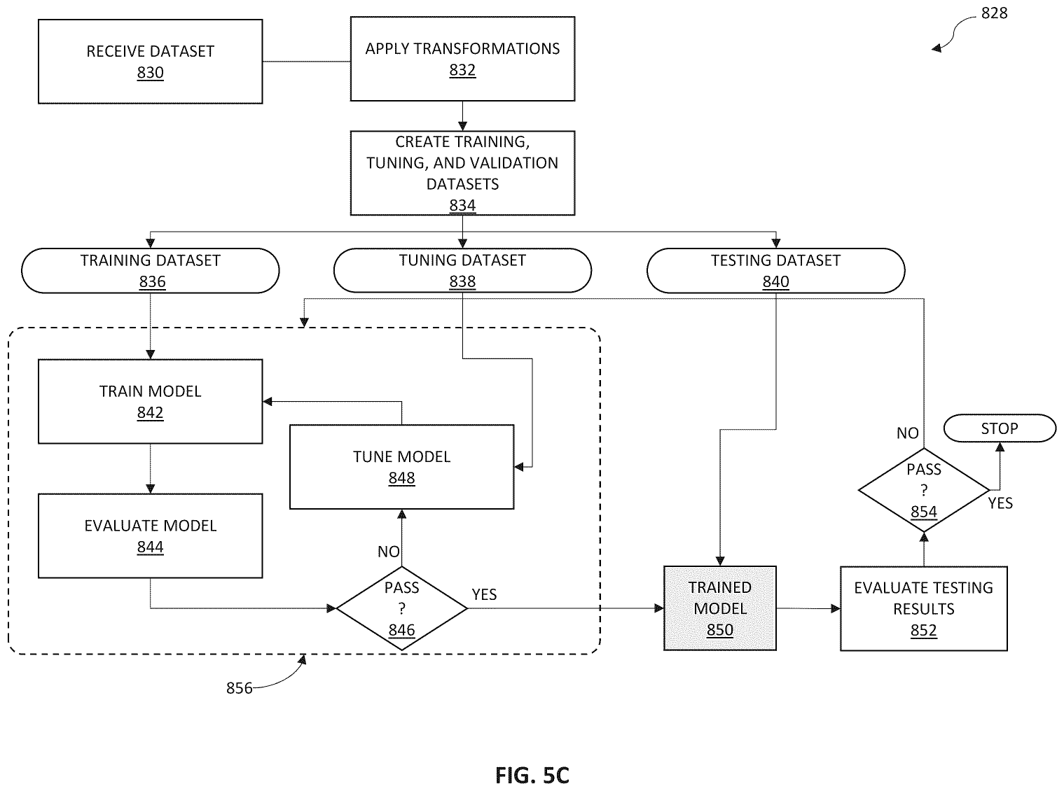

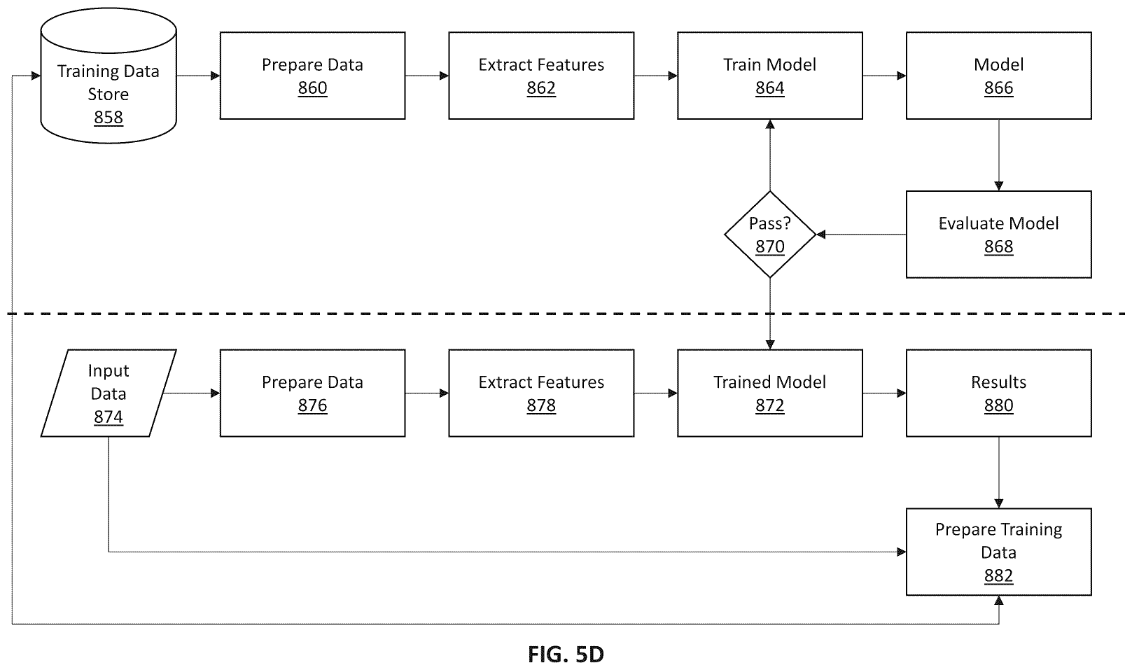

FIG. 5A is a block diagram that illustrates an example of a system and/or process 800 (both referred to here as a “system” for ease of reference) for identifying features and/or risk information of a patient using AI/ML based on non-invasively obtained medical images of the patient and/or patient information. In some embodiments, a current patient's medical data including images and/or patient information is first obtained and electronically stored on medical data storage 816 (e.g., cloud storage, hard disk, etc.). In some embodiments, the system 800 obtains medical images and/or patient information 818 from the medical data storage 816 and preprocess it, if necessary, for example to re-format it as necessary for further processing. The system 800 can also obtain a training set of medical images and/or patient information 822 from a stored dataset 820 of medical images and/or information of other patients (e.g., hundreds, thousands, tens of thousands, or hundreds of thousands or more of other patients). The medical images and information of other patients can be used to train the A I/M L algorithm 824 prior to processing the medical images and/or patient information 818 of the current patient, as described in further detail in reference to FIGS. 5C and 5D. In some embodiments, the AI/ML algorithm 824 can include one or more NN's, for example, as described in reference to the example NN illustrated in FIG. 6. The ML/AI 824 processes the medical images and/or patient information 818 of the current patient and generates outputs of identified features and/or risk information 826 of the current patient.

FIG. 5B is a schematic illustrating an example of a NN 812 that makes determinations 814 about characteristics of a (current) patient based on inputs that include medical images 802. In some embodiments, the NN 812 can be configured to receive other inputs 804. In some embodiments, the other inputs 804 can be medical images of other patients. In some embodiments, the other inputs 804 can be medical history of other patients. In some embodiments, the other inputs 804 can be medical history of the (current) patient. The NN 812 can include an input layer 806. In some embodiments, the NN 812 can be configured to present the training pattern to the input layer 806. In some embodiments, the NN 812 can include one or more hidden layers 808. In some embodiments, the input layer 806 can provide signals to the hidden layers 808, and the hidden layers 808 can receive signals from the input layer 806. In some embodiments, the hidden layers 808 can pass signals to the output layer 810. In some embodiments, one or more hidden layers 808 may be configured as convolutional layers (comprising neurons/nodes connected by weights, the weights corresponding to the strength of the connection between neurons), pooling layers, fully connected layers, and/or normalization layers. In some embodiments, the NN 812 may be configured with pooling layers that combine outputs of neuron clusters at one layer into a single neuron in the next layer. In some embodiments, max pooling and/or average pooling may be utilized. In some embodiments, max pooling may utilize the maximum value from each of a cluster of neurons at the prior layer. In some embodiments, back propagation may be utilized, and the corresponding neural network weights may be adjusted to minimize or reduce the error. In some embodiments, the loss function may comprise the Binary Cross Entropy loss function.

In some embodiments, the NN 812 can include an output layer 810. In some embodiments, the output layer 810 can receive signals from the hidden layers 808. In some embodiments, the output layer can generate determinations 814. In some embodiments, the NN 812 can make determinations 814 about characteristics of the patient. In some embodiments, the determinations 814 can include a characterized set of plaque. In some embodiments, the determinations 814 can include a patient's risk of CAD.

FIG. 5C depicts an example of a process in a flow diagram for training an artificial intelligence or machine learning model. The process 828 can be performed on a computing system. Various embodiments of such a process for training an AI or ML model can include additional features and/or CAN exclude certain illustrated features (for example, when a transformed/preprocessed dataset is received such that “apply transformations” in block 832 does not need to be performed.)

As illustrated in the example of FIG. 5C, at block 830 the system receives a dataset. At block 832, one or more transformations may be performed on the data in the dataset. In an example, data may require transformations to conform to expected input formats to conform with expected formatting, e.g., date formatting, units (e.g., pounds vs kilograms, Celsius vs Fahrenheit, inches vs centimeters, etc.), be of a consistent format, and the like. In some embodiments, the data may undergo conversions to prepare it for use in training an AI or ML algorithm, for example, categorical data may be encoded in a particular manner. In some embodiments, nominal data may be encoded using one-hot encoding, binary encoding, feature hashing, or other suitable encoding methods. In some embodiments, ordinal data may be encoded using ordinal encoding, polynomial encoding, Helmert encoding, and so forth. In some embodiments, numerical data may be normalized, for example by scaling data to a maximum of 1 and a minimum of 0 or −1. In some embodiments, a dataset can include images, and the images can undergo resizing, orienting, color correction, and so forth, color space transformations, and so forth. These are merely examples, and the skilled artisan will readily appreciate that other transformations are possible.

At block 834, the system may create, from the received dataset, training, tuning, and testing/validation datasets. In some embodiments, the training dataset 836 may be used during training to determine features for forming a model that can be used for prediction, classification, and so forth. In some embodiments, the tuning dataset 838 may be used to select final models (e.g., final model weights) and to prevent or correct overfitting that may occur during training with the training dataset 836, which can otherwise lead to poor generalization of the model. In some embodiments, the testing dataset 840 may be used after training and tuning to evaluate the model. For example, in some embodiments, the testing dataset 840 may be used to check if the model is overfitted to the training dataset. For example, when iterative training is used, overfitting can be indicated by continued improvement in the model performance on training data (e.g., the loss function or error continues to improve) while performance on a testing dataset improves for some period of time or number of training iterations, but then starts to decrease.

In some embodiments, the system, in training loop 856, may train the model at block 842 using the training dataset 836. In some embodiments, training may be conducted in a supervised, unsupervised, or partially supervised manner. In some embodiments of the present disclosure, supervised training may be used. At 844, in some embodiments, the system may evaluate the model according to one or more evaluation criteria. For example, in some embodiments, the evaluation may include determining how well the model can determine image transformations to account for changes in image acquisition parameters. At 846, in some embodiments, the system may determine if the model meets the one or more evaluation criteria. In some embodiments, if the model fails evaluation, the system may, at 848, tune the model using the tuning dataset 838, repeating the training 842 and evaluation 844 until the model passes the evaluation at 846. In some embodiments, once the model passes the evaluation at 846, the system may exit the model training loop 856. In some embodiments, the testing dataset 836 may be run through the trained model 842 and, at block 844, the system may evaluate the results. In some embodiments, if the evaluation fails, at block 846, the system may reenter training loop 856 for additional training and tuning. If the model passes, the system may stop the training process, resulting in a trained model 850. In some embodiments, the training process may be modified. For example, in some embodiments, the system may not use a tuning dataset 838. In some embodiments, the model may not use a testing dataset 840.

In some embodiments, testing can be performed within training loop 856, and training can be stopped once improvement in the model's performance on testing data stops improving or starts to decrease. For example, training can stop to avoid overfitting the model to the training data.

FIG. 5D illustrates an example of a process for training and using an AI/ML model. In some embodiments, training data store 858 can store data for training a model. For example, in some embodiments, training data store 858 can store a patient's medical images, as well as information about patient's physiology, such as weight, BMI, and so forth. At block 860, in some embodiments, a system can be configured to prepare the training data if it was not previously prepared for use in training a model. In some embodiments, as described briefly above, preparing the training data can include performing one or more normalization procedures, standardization procedures, and so forth, such as converting units (e.g., between Fahrenheit and Celsius, between inches and centimeters, between pounds and kilograms), converting dates to a standard format, converting times to a standard format, and so forth, processing images (e.g., size, orientation, color space, etc.). In some embodiments, it can be desirable to exclude certain data as additional data can consume additional computing resources and it can take longer to train a model. However, in some embodiments, exclusions may not be desirable as there can be a risk that a model may not accurately account for the influence of changes in image acquisition parameters on a resulting result. At block 862, the system can extract features from the training data and, at block 864, can train the model using the training data to produce model 866. At block 868, in some embodiments, the system can evaluate the model to determine if it passes one or more criteria. In some embodiments, at decision point 870, if the model fails, the system can perform additional training. In some embodiments, if, at decision point 870, the model passes, the system can make available trained model 872, which can be the model 872 after training is complete.

In some embodiments, the trained model 872 can be used to evaluate a particular patient. The input data 874 can relate to a specific input for which the outputs of the trained model 872 are desired. At block 876, the system can prepare the input data 874, for example as described above in relation to the stored training data. In some embodiments, at block 878, the system can extract features from the prepared user data. In some embodiments, the system can be configured to feed the extracted features to the trained model 872 to produce results 880.

In some embodiments, the input data 874, the results 880, and/or other information can be used to train the model. At block 882, in some embodiments, the system can prepare the input data 874 and the results 880 for use in training the model 872. In some embodiments, the system can store the prepared data in training data store 858. In some embodiments, the prepared data can be stored, additionally or alternatively, in another database or data store. In some embodiments, the system can retrain the model on periodically, continuously, or whenever an operator indicates to the system that the model should be retrained. Thus, in some embodiments, the trained model 872 can evolve over time, which can result in improved performance of the model (e.g., improved predictive capability, improved classification capability, and so forth) over time.

In some implementations, one or more machine learning models can be used for, for example, vessel extraction, aorta segmentation, vessel straightening, series ranking, coronary artery tree reconstruction, and so forth. A dataset used for training or testing can include, for example, CT images, coronary computer tomography angiography (CCTA) images, image acquisition parameters, and so forth.

In some embodiments, a machine learning model can be trained using supervised learning, where the training data includes one or more images as input and a desired output, such as a best series, a straightened vessel, a reconstructed coronary artery tree, and so forth. A representation of the input data (e.g., images) can be provided to the model. Output from the model can be compared to the desired output. For example, in a classification model, the desired output can be the true classification of the input, which can be compared with a classification determined by the model. In some embodiments, based on the comparison, the model can be modified, such as by changing weights associated with nodes of the neural network or parameters of the functions used at each node in the neural network (e.g., applying a loss function). The model can be modified until it produces the desired output with a desired accuracy.

### Computer System

In some embodiments, the systems, processes, and methods described herein are implemented using a computing system, such as the one illustrated in FIG. 6. The example computer system 928 is in communication with one or more computing systems 946 and/or one or more data sources 948 via one or more networks 944. While FIG. 5A illustrates an embodiment of a computing system 928, it is recognized that the functionality provided for in the components and modules of computer system 928 can be combined into fewer components and modules, or further separated into additional components and modules.

The computer system 928 can comprise a Plaque Analysis Module 940 that carries out the functions, methods, acts, and/or processes described herein. The Plaque Analysis Module 940 executed on the computer system 928 by a central processing unit 306 discussed further below.

In general the word “module,” as used herein, refers to logic embodied in hardware or firmware or to a collection of software instructions, having entry and exit points. Modules are written in a program language, such as JAVA, C#, C, or C++, or the like. Software modules can be compiled or linked into an executable program, installed in a dynamic link library, or can be written in an interpreted language such as JavaScript, BASIC, PERL, LUA, PHP, or Python and any such languages. Software modules can be called from other modules or from themselves, and/or can be invoked in response to detected events or interruptions. Modules implemented in hardware include connected logic units such as gates and flip-flops, and/or can include programmable units, such as programmable gate arrays or processors.

Generally, the modules described herein refer to logical modules that can be combined with other modules or divided into sub-modules despite their physical organization or storage. The modules are executed by one or more computing systems, and can be stored on or within any suitable computer readable medium, or implemented in-whole or in-part within special designed hardware or firmware. Not all calculations, analysis, and/or optimization require the use of computer systems, though any of the above-described methods, calculations, processes, or analyses can be facilitated through the use of computers. Further, in some embodiments, process blocks described herein can be altered, rearranged, combined, and/or omitted.

The computer system 928 includes one or more processing units (CPU) 932, which can comprise a microprocessor. The computer system 928 further includes a physical memory 936, such as random access memory (RAM) for temporary storage of information, a read only memory (ROM) for permanent storage of information, and a mass storage device 930, such as a backing store, hard drive, rotating magnetic disks, solid state disks (SSD), flash memory, phase-change memory (PCM), 3D X Point memory, diskette, or optical media storage device. Alternatively, the mass storage device can be implemented in an array of servers. Typically, the components of the computer system 928 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 928 includes one or more input/output (I/O) devices and interfaces 938, such as a keyboard, mouse, touch pad, and printer. The I/O devices and interfaces 938 can include one or more display devices, such as a monitor, which allows the visual presentation of data to a user. More particularly, a display device provides for the presentation of GUIs as application software data, and multi-media presentations, for example. The I/O devices and interfaces 938 can also provide a communications interface to various external devices. The computer system 928 can comprise one or more multi-media devices 934, such as speakers, video cards, graphics accelerators, and microphones, for example.

### Computing System Device/Operating System

The computer system 928 can run on a variety of computing devices, such as a server, a Windows server, a Structure Query Language server, a Unix Server, a personal computer, a laptop computer, and so forth. In other embodiments, the computer system 928 can run on a cluster computer system, a mainframe computer system and/or other computing system suitable for controlling and/or communicating with large databases, performing high volume transaction processing, and generating reports from large databases. The computing system 928 is generally controlled and coordinated by an operating system software, such as z/OS, Windows, Linux, UNIX, BSD, PHP, SunOS, Solaris, macOS, iOS, iPadOS, or other compatible operating systems, including proprietary operating systems and/or open source operating systems. Operating systems control and schedule computer processes for execution, perform memory management, provide file system, networking, and I/O services, and provide a user interface, such as a graphical user interface (GUI), among other things.

### Network

The computer system 928 illustrated in FIG. 6 is coupled to a network 944, such as a LAN, WAN, or the Internet via a communication link 942 (wired, wireless, or a combination thereof). Network 944 communicates with various computing devices and/or other electronic devices. Network 944 is communicating with one or more computing systems 946 and one or more data sources 948. The Plaque Analysis Module 914 can access or can be accessed by computing systems 946 and/or data sources 948 through a web-enabled user access point. Connections can be a direct physical connection, a virtual connection, and other connection type. The web-enabled user access point can comprise a browser module that uses text, graphics, audio, video, and other media to present data and to allow interaction with data via the network 944.

The output module can be implemented as a combination of an all-points addressable display such as a cathode ray tube (CRT), a liquid crystal display (LCD), a plasma display, or other types and/or combinations of displays. The output module can be implemented to communicate with input devices 938 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module can communicate with a set of input and output devices to receive signals from the user.

### Other Systems

The computing system 928 can include one or more internal and/or external data sources (for example, data sources 948). In some embodiments, one or more of the data repositories and the data sources described above can be implemented using a relational database, such as DB2, Sybase, Oracle, CodeBase, and Microsoft® SQL Server as well as other types of databases such as a flat-file database, an entity relationship database, and object-oriented database, and/or a record-based database.

The computer system 928 can also access one or more databases 948. The databases 948 can be stored in a database or data repository. The computer system 928 can access the one or more databases 948 through a network 944 or can directly access the database or data repository through I/O devices and interfaces 938. The data repository storing the one or more databases 948 can reside within the computer system 928.

### URLs and Cookies

In some embodiments including any of the embodiments disclosed herein (above or below) one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name, and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

### Invasive Diagnostic Techniques

Evaluating patients often involves the use of invasive techniques. One such invasive technique is fractional flow reserve (FFR). FFR is a diagnostic technique used in cardiology to measure pressure differences across a coronary artery stenosis. FFR can be used to assess the likelihood that stenosis impedes oxygen delivery to the heart muscle. FFR can be especially useful for determining the functional significance of coronary artery disease (CAD). FFR can be used to help determine, for example, whether a stenosis would benefit from revascularization (e.g., angioplasty or coronary artery bypass grafting) or is better managed by non-invasive medical therapy alone. FFR can lead to improved patient outcomes, including, for example, reduced risk of major adverse cardiac events (MACE). FFR can also reduce healthcare costs by avoiding unnecessary procedures, such as revascularization in patients for whom medical therapy alone is sufficient.

However, FFR has significant drawbacks. For example, FFR is an invasive procedure that involves insertion of a catheter into the coronary artery, for example via the femoral or radial artery. A pressure-sensing wire is advanced through the catheter to the site of stenosis. A hyperemic agent, such as adenosine, is administered to the patient to maximize bloodflow and create a state of hyperemia. Pressure can then be measured proximal and distal to the site of stenosis. FFR can then be calculated as the ratio of distal to proximal pressure. Typically, an FFR value of less than 0.8 or about 0.8 indicates that the stenosis is functionally significant and may benefit from revascularization.

Instantaneous Wave-Free Ratio (iFR) is another diagnostic technique that can be used to assess the severity of coronary artery stenosis. iFR can provide a functional assessment of CAD without the need for inducing hyperemia. iFR measurements are taken in a manner similar to FFR, using a catheter and pressure wire. However, iFR measurements are taken during the wave-free period of the cardiac cycle, for example, in diastole when the resistance in the coronary arteries is at its lowest and most stable.

### FFR3D

To address challenges and limitations associated with invasive FFR measurements, non-invasive techniques have been developed that aim to provide similar information, for example using advanced imaging and computational techniques. One such technique is FFR-CT. FFR-CT is a non-invasive imaging technique that combines coronary computed tomography angiography (CCTA) with computational fluid dynamics to estimate FFR values. FFR-CT can provide a functional assessment of coronary artery disease without the need for invasive catheterization. In FFR-CT, a patient undergoes a CCTA scan, which can provide detailed images of the coronary arteries. The CCTA images can be processed and used to simulate bloodflow and pressure within the coronary arteries. Software can be used to calculate FFR values along the coronary arteries, which can help to identify areas where bloodflow is significantly reduced.

FFR-CT can be used for initial assessment, for example, of patients suspected to have CAD. When severity of stenosis is unclear from CCTA, FFR-CT can help determine the functional significance of lesions. FFR-CT can also be suitable for use when patients are at high risk of complications from invasive procedures, which can weigh against performing a traditional FFR procedure.

Typically, FFR-CT uses computational fluid dynamics (CFD) methods, which are complex, computationally intensive, and time-consuming. To make CFD calculations feasible (e.g., taking an acceptable amount of time and/or using an acceptable amount of computational resources), often various assumptions are made. For example, CFD calculations may assume steady-state flow, treat blood as a Newtonian fluid, treat vessel walls as rigid, apply simplified boundary conditions such as inlet and outlet pressures and flow rates, and so forth. CFD calculations often either do not include microcirculation in small blood vessels and capillaries or attempt to approximate the effects of microcirculation. FFR-CT calculations typically assume laminar flow, linear pressure drops across stenoses, no collateral circulation, the same hyperemic flow for all patients, the same microvascular resistance for everyone, the same blood pressure for everyone, and/or that artery size is directly proportional to myocardial mass.

While FFR-CT can provide many benefits, FFR-CT may be less accurate than invasive FFR, particularly in patients with complex coronary anatomy or severe calcification, where the assumptions made in FFR-CT calculations may be inaccurate and result in incorrect FFR values. Moreover, even in healthy patients, FFR-CT can produce inaccurate FFR values. For example, errors in assumptions can be multiplicative, resulting in large errors when there are erroneous assumptions. Another limitation of FFR-CT is its deterministic nature. That is, equations used in FFR calculations are fixed, and new variables cannot be easily added. FFR-CT can produce high precision FFR estimates, but FFR-CT estimates may be inaccurate in many cases.

Thus, there is a need for improved approaches that can provide reliable, actionable FFR values without the need for invasive FFR measurements. Described herein are approaches to non-invasively determine FFR values. The approaches herein, collectively and individually referred to herein as FFR 3D, can utilize 3D-printed models of coronary arteries and various computational techniques to determine FFR values for a subject. FFR3D as described herein can provide several benefits over FFR-CT and/or other invasive and/or non-invasive diagnostic techniques. In some embodiments, FFR 3D can act as an alternative to invasive FFR and/or iFR. In some embodiments, FFR3D approaches as described herein are used to determine prescribed flow reserve (e.g., ischemia at a patient's level of activities for daily living), hyperemic stenosis resistance (HSR) index, mean blood flow (MBF), coronary flow reserve (CFR), CFR-FFR mismatch, and/or wall shear stress. FFR3D, as described herein, may be more broadly applicable to a population. For example, FFR3D as described herein may not rely on invasive FFR measurement, which typically are only captured for subjects with coronary artery disease. Rather, FFR3D can utilize measurements that represent a wide variety of health states, from healthy subjects to those with coronary artery disease.

Building a 3D model of a subject's coronary arteries, printing the model, and subsequently performing fluid flow measurements using the model can be an effective way to identify clinically significant stenoses in patients. However, performing 3D printing and physical measurements at individual subject level can be resource and/or time-intensive, involving significant materials consumption, a large time investment as 3D printing can take hours to complete and conducting measurements also takes significant time, and so forth. Accordingly, it may not be feasible to 3D print the arteries of each patient. However,

In some embodiments, data is collected using 3D models (e.g., physical 3D models) that can be used to determine FFR3D values. For example, various fluid flow properties such as flow rate, pressure, tortuosity, etc., can be set and/or measured in a 3D printed model and used to determine FFR values. As described herein, some embodiments can utilize one or more machine learning models. However, some other embodiments can operate without the use of machine learning models. Some embodiments utilize relationships between fluid flow properties (e.g., pressure, flow rate, etc.) as inputs into a machine learning model, either with or without anatomical information about a subject's coronary anatomy.

In some embodiments, data can be collected using 3D models (e.g., physical 3D models) that can be used to determine FFR 3D values. For example, various fluid flow properties such as flow rate, pressure, tortuosity, etc., can be set and/or measured in a 3D printed model and used to determine FFR 3D values. As described herein, some embodiments can utilize one or more machine learning models. However, some other embodiments can operate without the use of machine learning models.

In some embodiments, machine learning models can be used to automatically segment coronary arteries from CCTA images, for example by identifying coronary artery boundaries. A machine learning model can be trained to extract anatomical features from CCTA images. For example, a machine learning algorithm can identify one or more regions of plaque and/or one or more lesions in a vessel. A machine learning algorithm can be used to determine variables such as stenosis (e.g., percent diameter stenosis), total plaque volume, non-calcified plaque volume, calcified plaque volume, low attenuation plaque volume, lesion length, remodeling index, plaque slice percentage, stenosis area percentage, presence of low-density plaque, stenosis diameter percentage, presence of positive remodeling, reference diameter after stenosis, reference diameter before stenosis, vessel length, lumen volume, number of chronic total occlusions (CTOs), vessel volume, number of stenoses, and/or number of mild stenoses.

In some embodiments, a machine learning model can obviate the need for CFD calculations or for an ML-based surrogate for CFD calculations. For example, in some embodiments, a machine learning model can be trained to predict FFR values directly from CCTA images. For example, a model can be trained to learn the complex relationships between image features and FFR values. In some embodiments, a machine learning model can combine anatomical features, geometric features, and/or physiological measurements (e.g., pressure pullback curves) to determine FFR values.

A significant challenge associated with using machine learning models for FFR is the potential lack of accuracy, lack of generalizability, etc., of such models. For example, ML model performance depends on the quality, quantity, and diversity of training data. However, obtaining a diverse set of high quality FFR data and CCTA images can be challenging. For example, FFR is typically only done on patients with suspected CAD and collected data may not be generalizable to the entire population. Moreover, even patients with suspected or diagnosed CAD may not undergo FFR, for example, due to cost, lack of availability, risks associated with FFR procedures, and so forth. Additionally, data may not always be of high quality. For example, values can depend upon the accuracy of human readers, which can have significant variability. For example, FFR measurements may be accurate, but the determined geometry of the arteries may be inaccurate. Thus, a data set that relies on actual patient data may be limited in size, diversity, quality, etc. As a result, machine learning models for use in FFR value determination may be of limited utility and may even produce values with significant errors in some cases.

In some embodiments, as described herein, a machine learning model can be trained using data that is more accurate, more precise, more diverse, larger, or any combination thereof. Rather than or in addition to training a machine learning model using real patient data, 3D models of patients can be generated, and data can be collected for such models. For example, the flow properties of fluids in 3D printed models of coronary arteries can be determined and used to train a machine learning model to make FFR predictions.

Such an approach can have many advantages. For example, vessel geometry (e.g., length, curvature, diameter, etc.) can be more accurately determined than may be possible when using real patient data in which vessel geometry is determined via imaging alone. That is, for example, the geometry of a 3D printed vessel can be known more accurately as the 3D printed vessel has a well-defined, known geometry based on the 3D representation used to print the vessel and/or the tolerances of the 3D printer used to print the vessel. It can be easier to generate data for a wide variety of vessel geometries, as curves, dimensions, and so forth can be readily varied by printing modified 3D models. In some embodiments, variations of 3D models can be generated automatically. In some embodiments, variations can be generated within certain constraints, for example to ensure that generated 3D models represent possible and/or likely real patient coronary artery vessels. Generating new 3D models can be relatively easy, as doing so may only involve updating a 3D model file and printing a new 3D model, as opposed to performing imaging and invasive FFR on a real patient. Thus, a large volume of diverse, high quality data can be obtained with relative ease.

Information derived from 3D printed models can be used to train a machine learning model or to build a physical model to determine FFR values and/or to determine other values for use in calculating FFR values.

In some embodiments, a single ML model is used for determining FFR values in a patient-agnostic manner. However, it will be appreciated that there can be significant differences between subpopulations within a population. For example, different geographic areas, races, socioeconomic groups, and so forth may have different exposures, behaviors, and so forth that can impact cardiac health. In some embodiments, multiple models can be generated for different populations. For example, models can be generated at a global level, national level, state or province level, county level, city level, metropolitan area level, combined statistical area level, etc. This can be significant as people in different locations may have access to different foods, transit options (e.g., people who live in dense cities may be more likely to walk than someone who lives in a suburb or rural area and relies on a personal automobile for transportation), and so forth. Altitude can significantly impact cardiovascular function, and people who live in higher altitudes may progress differently than those who live at lower altitudes. Weather can impact how much outdoor activity individuals engage in. Areas with certain industries may tend to present with somewhat different pathologies. For example, residents in a mining town—and particularly male residents—may be more likely to suffer the effects of poor air quality and exposure to contaminants in mines. Similarly, people who live close to polluting factories or open air mines may inhale particulates, chemicals, etc., that have adverse health impacts. In some embodiments, models are generated for particular clinics or practitioners. Such models may be especially beneficial for clinics or physicians that tend to treat one type of patient (or a few types of patients), such as a clinic that focuses on treatment of conditions in retired coal miners.

Models can be generated based on CCTA scans for a limited number of subjects in a particular population. For example, a model can be generated using data from 10 subjects, 20 subjects, 50 subjects, 100 subjects, or any other suitable number of subjects. In some embodiments, as described herein, additional data points for training a model are generated using models that are based on CCTA scan data and modified, for example to change lumen diameter, curvature, plaque volume, plaque shape, plaque composition, etc.

In some embodiments, models are static. However, in some embodiments, models undergo continuous learning, periodic retraining, or both. For example, patients who come in for imaging can be asked to consent to having their CCTA scans used for machine learning model training, and CCTA scans for patients who provide consent can be used to update a machine learning model. Updates can be made on various schedules, such as quarterly, annually, etc., on an ad hoc basis, or both.

Past research using 3D models has relied largely on idealized stenoses rather than patient-specific coronary anatomy. Such idealized stenoses fail to account for real-world stenoses which can have irregular shapes, differing lengths, different compositions (e.g., differing levels of calcified plaque, non-calcified plaque, low attenuation plaque, etc., which can impact how stenoses behave). Such models can also fail to account for effects of multiple stenoses, in which multiple stenoses contribute to ischemia. Additionally, such research has typically focused on single vessels, while in real patients, stenoses in one vessel can influence ischemia in other vessels. To be a suitable replacement for invasive FFR, FFR as determined using 3D models (FFR 3D) can be based on measurements across a range of physiologically realistic boundary conditions.

According to some embodiments as described herein, the techniques herein can provide FFR 3D values that a more accurate and reliable. FFR 3D values as determined using the techniques herein can be personalized based on a patient's anatomy, levels of physical activity, etc. FFR3D values can be based upon actual measurements, such as actual measurements of the patient's coronary anatomy (e.g., vessel diameter, lumen diameter, plaque geometry, etc.). In some embodiments, the patient's coronary anatomy can be determined from CCTA images, for example using a machine learning model configured to process CCTA images and extract coronary anatomy information. In some embodiments, the techniques herein can account for arterial wall material properties (e.g., calcified plaque, non-calcified plaque, low attenuation plaque, etc.). In some embodiments, the techniques herein can allow for flexible boundary conditions, such as changing input blood pressure, flow velocity, and so forth.

While FFR is often assumed to be unaffected by physiological conditions, it has been observed that FFR values can be susceptible to factors such as aortic blood flow, blood flow velocity, microcirculatory resistance, heart rate, etc. For example, it has been observed that there is a quadratic relationship between FFR values and blood flow velocity. The relationship between FFR and blood flow rate can depend on the percent diameter stenosis (e.g., the reduction in the diameter of a vessel at the site of stenosis as compared with a normal, healthy segment of the vessel), with a blood flow rate having a much larger impact on pressure ratios (Pd/Pa) for higher percent diameter stenosis.

In some embodiments, the techniques herein can be used to enable analysis such as absolute myocardial blood flow (MBF), which can help to explain mismatches between FFR and coronary flow reserve (CFR). For example, low FFR with normal CFR can indicate compensatory microvascular function, or normal FFR with low CFR can indicate microvascular dysfunction; hyperemic stenosis resistance at normal vs high coronary flow velocity states, prescribed flow reserve (e.g., personalized FFR based upon expected coronary flow velocities from a patient's daily activities); microcirculatory resistance; microvascular resistance; wall shear stress; etc.

Various parameters can be fixed, varied, measured, assumed, or calculated. For example, in some embodiments, the techniques described herein include determining a relationship between pressure drop (e.g., distal pressure (Pd)-proximal pressure (Pa)) and blood flow (Q). In some embodiments, Pa can be assumed or fixed, and Pd can be measured while Q is varied. In some embodiments, FFR (Pd/Pa) can be calculated while Q is varied. In some embodiments, Pd can be measured, and FFR 3D values can be calculated as a function of CFR, which can be assumed.

In some embodiments, pressure pullback curves can be measured in 3D printed models, similar to how invasive FFR procedures are performed in vivo. These values can be used to determine FFR values.

While 3D printed models can offer many advantages, for example, as described herein, there can also be several challenges associated with using 3D printed models instead of FFR data from real patients. For example, a 3D printed model may not behave in the same way as real coronary vessels, which are capable of deforming, expanding, and contracting.

While some embodiments utilize machine learning models, it is not necessary to use a machine learning model for FFR 3D. In some embodiments, the techniques described herein may not make use of machine learning. For example, the techniques herein can analyze fluid flow properties in 3D printed models and determine a relationship between one or more of the fluid flow properties and FFR values.

While 3D models can provide many benefits, there are also some complications associated with using 3D models to analyze circulation. The flexibility and/or movement of coronary arteries plays an important role in maintaining proper blood flow to the heart muscle. When the coronary arteries widen (vasodilation), the increased diameter of the vessels allows more blood to flow through. Conversely, when the arteries narrow (vasoconstriction) the decreased diameter of the vessels decreases blood flow. The coronary arteries are elastic, meaning they can stretch and then return to their original shape. This elasticity helps to maintain a continuous flow of blood even when the heart is in between beats (diastole). While the elasticity of the blood vessels can be important, such elasticity can be difficult to replicate in a 3D printed material.

Additionally, 3D printed vessels typically remain in a static shape. However, during normal function of the heart, the vessels move as the heart beats. Changes in the shape of the vessels can have a significant impact on bloodflow. Curves, kinks, bends, and so forth in vessels can cause disruptions in fluid flow, leading to increased turbulence, energy losses due to friction and changes in momentum, and so forth. Such disruptions can, for example, result in pressure drops across the vessel and/or otherwise reduce flow efficiency. As the vessels change shape, the presence and/or severity of such curves, kinks, bends, etc., can vary, resulting in different bloodflow characteristics throughout the cardiac cycle.

Thus, it can be important to account for differences in the physical behavior of 3D printed vessels as compared with real coronary vessels. In some embodiments, different materials (e.g., materials with different flexibility, elasticity, etc.), different material densities, etc., can be used in a 3D printing process so that the mechanical properties of 3D-printed vessels more closely represent those of in vivo vessels. In some cases, there can still be differences between the behavior of a fluid in 3D printed vessels and in vivo vessels.

Additionally, replicating the fluid behavior of blood can be challenging. For example, blood is generally considered a non-Newtonian fluid. For example, red blood cells can aggregate at low shear rates and disaggregate at higher shear rates, resulting in changes in viscosity.

In some embodiments, a machine learning model can be tuned based on a comparison of modeled FFR values and FFR values determined from invasive FFR. Such an approach can be used to build a model that is more widely generalized, more accurate, etc., than a model trained using only real patient CT scans and invasive FFR data as a larger amount of data, higher quality data, etc., can be used in training the model, but invasive FFR data, when available, can be used to tune the model so that it more accurately reflects in vivo bloodflow through coronary vessels.

In some embodiments, there can be static offsets between modeled FFR values and invasive FFR values. Static offsets can be a single value or multiple values. That is, static offsets may differ based on various properties, flow rates, pressures, etc. By comparing modeled and invasive FFR values across a large number of coronary arteries, an offset table or function can be defined. The offset table or function can be used to adjust modeled FFR values so that they more accurately reflect what would be observed in invasive FFR.

In some embodiments, FFR3D values are determined using blood flow values. For example, there can be a quadratic relationship between FFR and blood flow velocity.

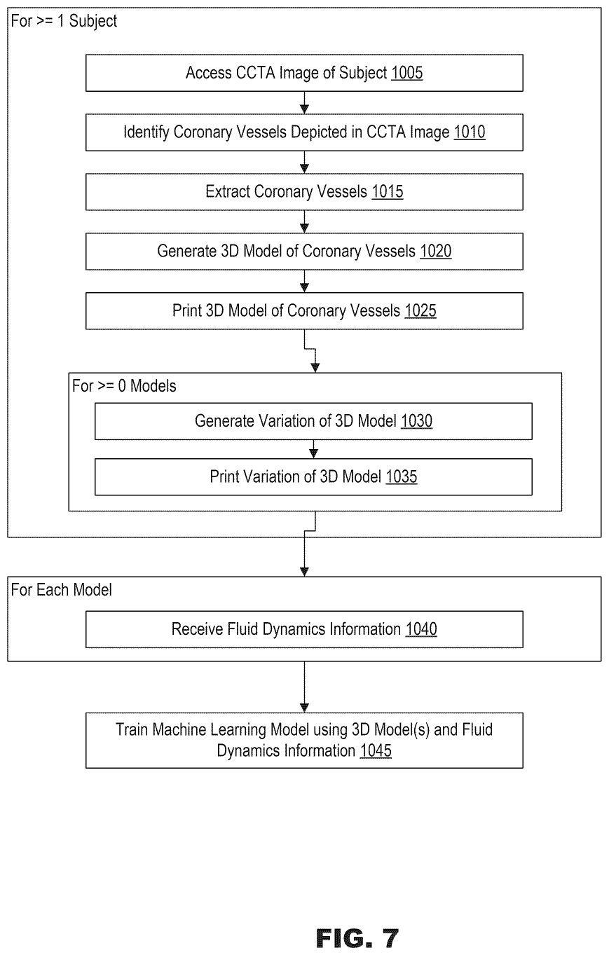

FIG. 7 is a flowchart that illustrates an example process for generating data for a machine learning model and training a machine learning model according to some implementations. The process illustrated in FIG. 7 can be performed using a computer system or multiple computer systems. For each of one or more subjects, the system can access a CCTA image of the subject at operation 1005. At operation 1010, the system can identify coronary vessels depicted in the CCTA image. At operation 1015, the system can extract the coronary vessels. At operation 1020, the system can generate a 3D model of the coronary vessels, for example based on the extracted coronary vessels. The 3D model can be a model that is suitable for 3D printing. For example, the 3D model can be represented by a file such as STL (Stereolithography), OBJ (Object File), AMF (Additive Manufacturing File Format), 3M F (3D Manufacturing Format), PLY (Polygon File Format), VRML (Virtual Reality Modeling Language), G-code, FBX (Filmbox), or any other suitable file format. In some embodiments, the system is configured to generate 3D model files in a particular format. In some embodiments, the system is configured to generate 3D model files in one or more of a variety of formats, for example, based on a target output device (e.g., 3D printer). At operation 1025, the system can cause a 3D printer to print the 3D model, for example by creating a print job and transmitting the print job to the 3D printer.

As described herein, in some embodiments, 3D models can be varied to generate additional 3D models. Operations 1030 and 1035 can be carried out for zero or more models for zero or more subjects. At operation 1030, the system can generate a variation of the 3D model, and can cause printing of the variation of the 3D model at operation 1035.

The 3D printed models can be used to collect fluid dynamics information, such as flow rate, pressure, and so forth. In some embodiments, the information can include pressure pullback curves, for example as determined using a catheter and pressure sensor that is pulled through the 3D printed vessels. At operation 1040, the system can receive fluid dynamics information captured using one or more printed 3D models. At operation 1045, the system can train a machine learning model using 3D model information (e.g., a description of the 3D models contained in 3D model files) and the corresponding fluid dynamics information.

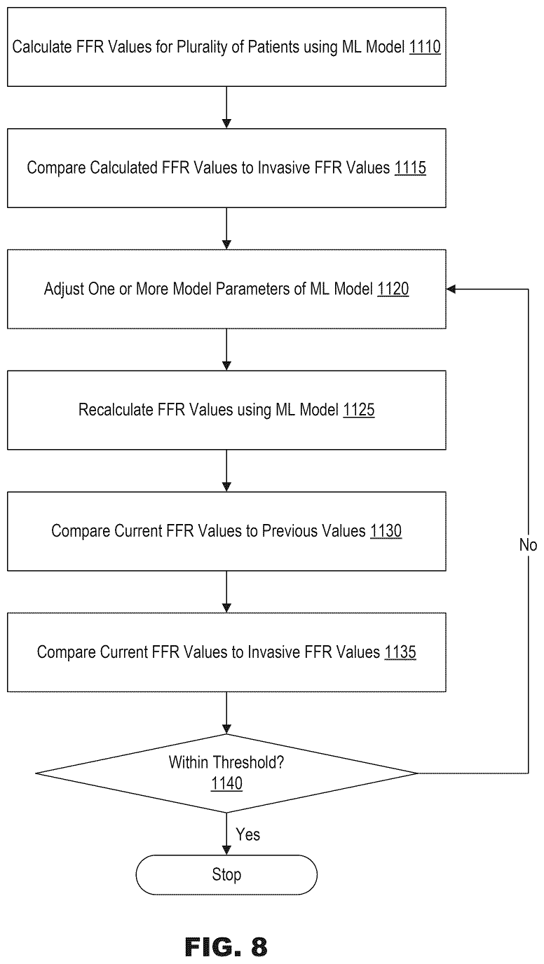

FIG. 8 is a flowchart that illustrates an example process for adjusting a machine learning model according to some embodiments. At operation 1110, a system can calculate FFR values for a plurality of patients using the M L model. At operation 115, the system can compare the calculated FFR values to invasive FFR values for the patients. At operation 1120, the system can adjust one or more model parameters of the ML model. At operation 1125, the system can recalculate FFR values for the patients using the ML model. At operation 1130, the system can compare the updated FFR values to the previous values. At operation 1135, the system can compare the updated FFR values to the invasive FFR values. The comparison between the updated FFR values and the previous FFR values can indicate directionality of the changes in the FFR values resulting from the modification of the model parameters. Comparison to the invasive FFR values can indicate if the ML-derived values are closer or further from the invasive FFR values. At operation 1140, the system can determine if the updated FFR values are within a threshold amount from the invasive FFR values. If so, the process can stop. If not, the system can proceed to operation 1120 and adjust one or more model parameters of the ML model.

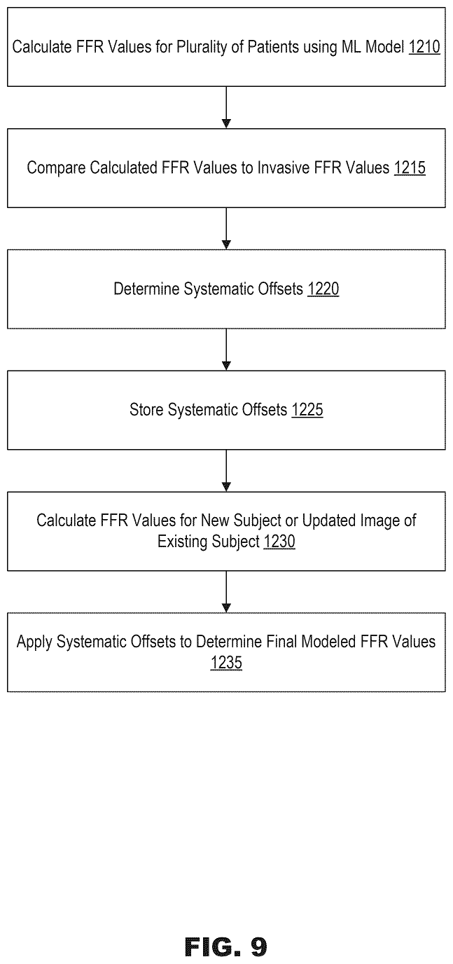

FIG. 9 is a flowchart that illustrates an example process for determining systematic offsets that can be applied to the outputs of a machine learning model according to some embodiments. At operation 1210, a system can calculate FFR values for a plurality of patients using the ML model. At operation 1215, the system can compare the calculated FFR values to invasive FFR values. At operation 1220, the system can determine systematic offsets between the calculated FFR values and the invasive FFR values. At operation 1225, the system can store the systematic offsets for future use. At operation 1230, the system can calculate FFR values for a new subject (or a new image for an existing subject) using the ML model. At operation 1235, the system can apply systematic offsets to the calculated FFR values to determine final modeled FFR values.

Various methods can be used to determine relationships between FFR values determine from 3D-printed vessels (referred to as FFR3D values) and invasive FFR values. For example, FFR3D values can be determined on a per-vessel, per-segment, or per-unit-distance (e.g., per-millimeter) basis. FFR3D values can be determined using only physics principles, only machine learning, or a combination of both.

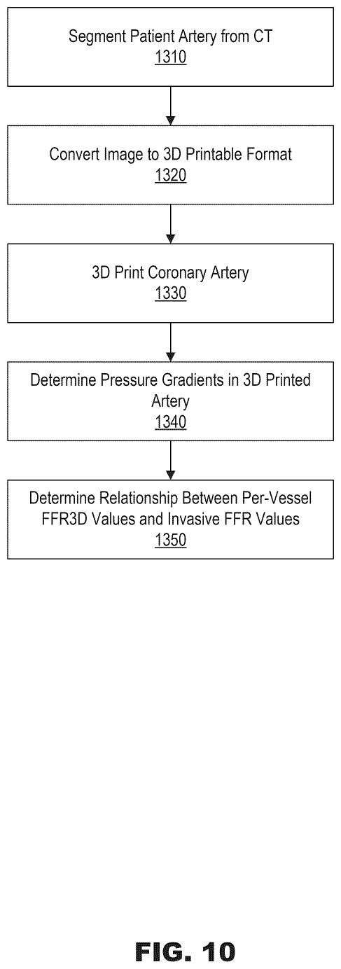

FIG. 10 is a flowchart that illustrates an example physics-based, per-vessel approach for mapping FFR 3D and FFR values according to some embodiments. At operation 1310, a system can segment patient arteries from one or more CT images. At operation 1320, the system can convert the image to a 3D printable format. For example, the system can extract coronary vessels from the CT images and generate a 3D model such as an STL file that can be 3D printed. At operation 1330, the system can 3D print the coronary vessels. For example, the system can instruct a 3D printer to print the artery using a 3D printer. At operation 1340, the system can determine pressure gradients in the 3D-printed vessels. The system can use the determined pressure gradients to generate FFR 3D values. At operation 1350, the system can determine a relationship between the FFR3D values and invasive FFR values on a per-vessel basis. In some embodiments, the relationship can be a quadratic relationship. In some embodiments, only a single vessel is 3D printed. In some embodiments, multiple vessels are 3D printed. In some embodiments, all or substantially all of a coronary artery tree is 3D printed.

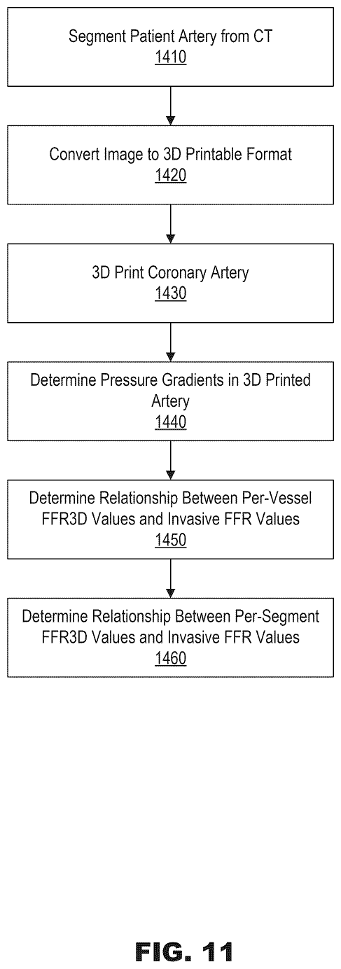

FIG. 11 is a flowchart that illustrates an example physics-based, per-segment process for mapping FFR 3D and FFR values according to some embodiments. At operation 1410, a system can segment patient coronary vessels from one or more CT images. At operation 1420, the system can generate a representation of the vessels in a 3D printable file format. At operation 1430, the system can 3D print the coronary vessels. At operation 1440, the system can determine pressure gradients in the 3D-printed vessels, which can be used to determine FFR 3D values. At operation 1450, the system can determine a relationship between per-vessel FFR 3D values and invasive FFR values. At operation 1460, the system can determine per-segment relationships between FFR3D values and invasive FFR values.

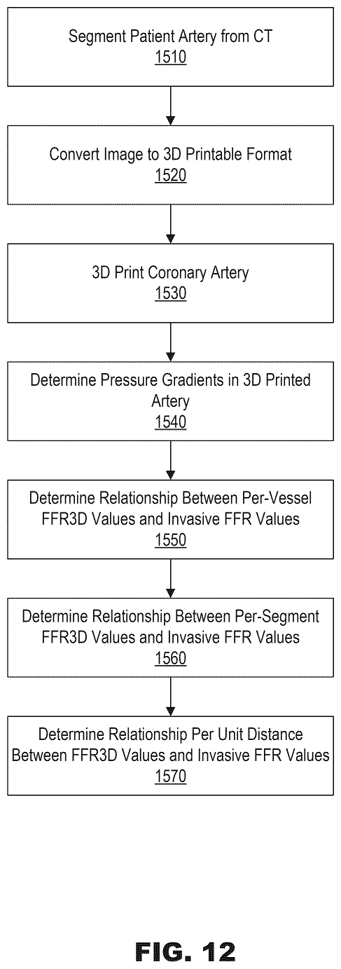

FIG. 12 is a flowchart that illustrates an example physics-based, per-unit-length process for mapping FFR 3D and FFR values according to some embodiments. At operation 1510, a system can segment a subject's coronary vessels from CT images. At operation 1520, the system can generate a 3D-printable file of the coronary vessels. At operation 1530, the system can 3D print the coronary vessels. At operation 1540, the system can determine pressure gradients in the 3D printed coronary vessels. At operation 1550, the system can determine per-vessel relationships between FFR 3D values and invasive FFR values. At operation 1560, the system can determine per-segment relationships between FFR 3D values and invasive FFR values. At operation 1570, the system can determine per-unit-distance FFR 3D values and invasive FFR values. The unit distance can be, for example, 0.5 mm, 1 mm, 1.5 mm, 2 mm, or any other distance.

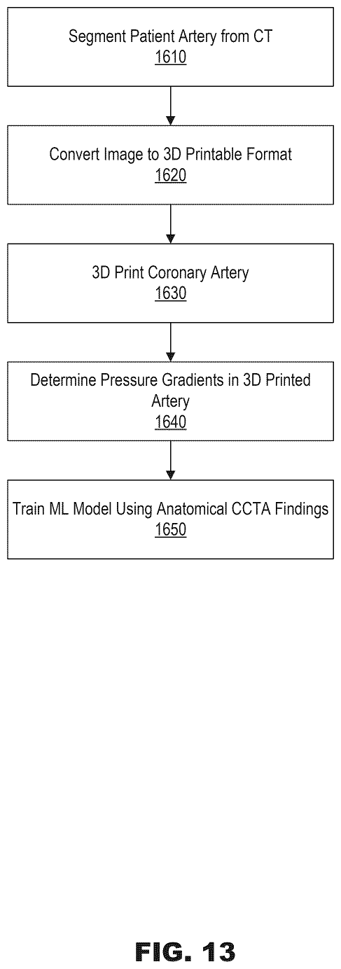

FIG. 13 is a flowchart that illustrates an example process for training a machine learning model to generate FFR3D values according to some embodiments. In the process of FIG. 13, only anatomical data is used as an input to the machine learning model. At operation 1610, a system can segment coronary vessels from one or more CT images. At operation 1620, the system can generate a 3D printable file from the segmented vessels. At operation 1630, the system can 3D-print the vessels. At operation 1640, the system can determine pressure gradients in the 3D printed vessels. At operation 1650, the system can train a machine learning model. The machine learning model can be trained using anatomical CCTA findings as inputs. The anatomical CCTA findings can indicate, for example, lumen diameter, curvature, and so forth, for example as described herein. The machine learning model can be trained using supervised learning, in which the machine learning model is trained to reproduce the determined pressure gradients (or FFR3D values determined from the pressure gradients).

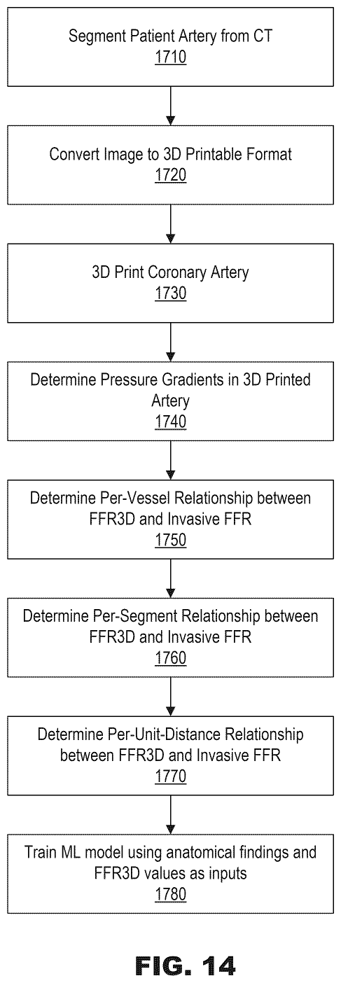

FIG. 14 is a flowchart that illustrates an example process that combines physics-based and anatomical-based approaches according to some embodiments. At operation 1710, a system can segment coronary vessels from one or more CT images. At operation 1720, the system can generate a 3D printable file representing the coronary vessels. At operation 1730, the system can 3D print the coronary vessels. At operation 1740, the system can determine pressure gradients in the 3D-printed coronary vessels. At operation 1750, the system can determine per-vessel relationships between FFR3D and invasive FFR values. At operation 1760, the system can determine per-segment relationships between FFR 3D and invasive FFR values. At operation 1770, the system can determine per-unit-distance relationships between FFR 3D and invasive FFR values. At operation 1780, the system can train a machine learning model using anatomical findings and FFR3D values as inputs, and can train the model to reproduce the determined pressure gradients.

As another example, in some embodiments, a 3D printed model can include embedded barbs and pressure sensors that allow for direct measurement of pressure across a range of physiologically realistic boundary conditions. In some embodiments, a patient can undergo a CT scan (e.g., a CCTA scan), and the resulting CCTA image can be used to 3D print a representation of the patient's vessels. The 3D printed representation can be used to measure FFR values for that correspond to the patient without the use of a model.

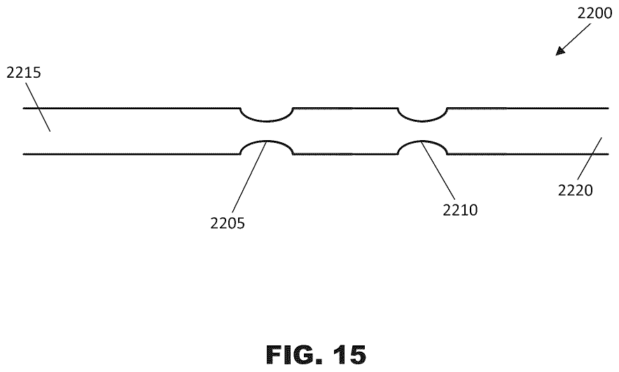

FIG. 15 is a drawing that illustrates idealized stenoses and pressure within a vessel at various locations. In FIG. 15, a vessel 2200 includes a first stenosis 2205 and a second stenosis 2210. The vessel has an inlet 2215 and an outlet 2220. The first stenosis 2105 can reduce the vessel diameter from normal diameter r_{NORM} to a first restricted diameter r_{S1}, and the second stenosis 2210 can reduce the vessel diameter from r_{NORM} to r_{S2}. The stenoses may have different diameters, lengths, and so forth. There can be a pressure drop across the first stenosis 2205, which can recover and then drop again across second stenosis 2210. The pressure difference across the stenoses (Pa-Pd)/Pa can define an FFR value.

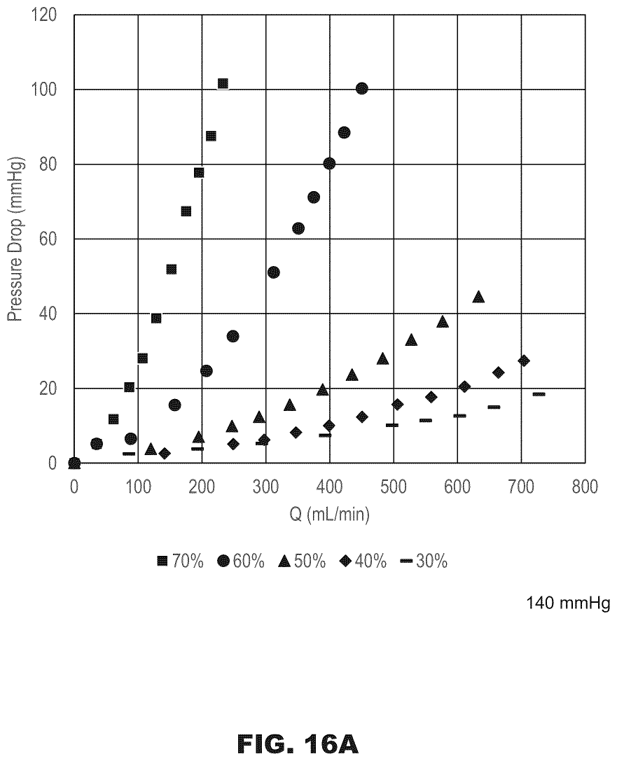

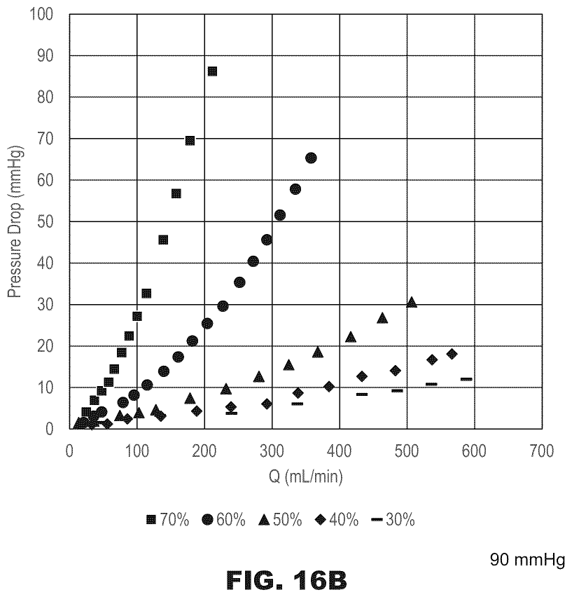

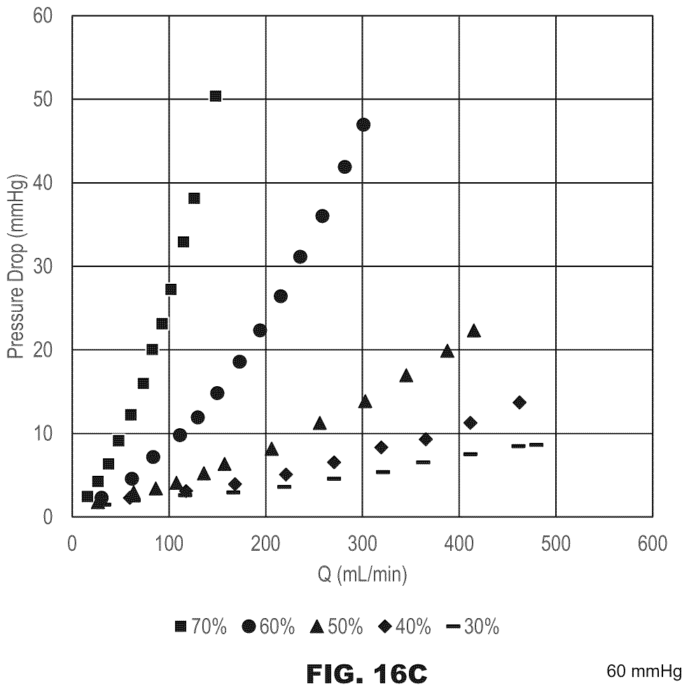

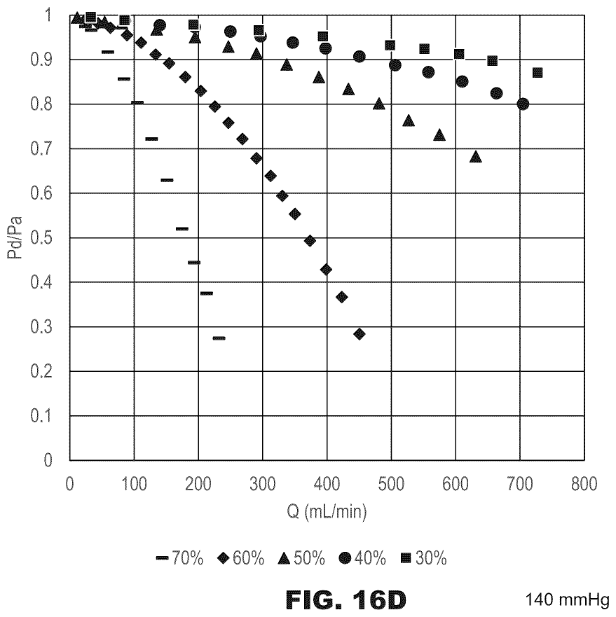

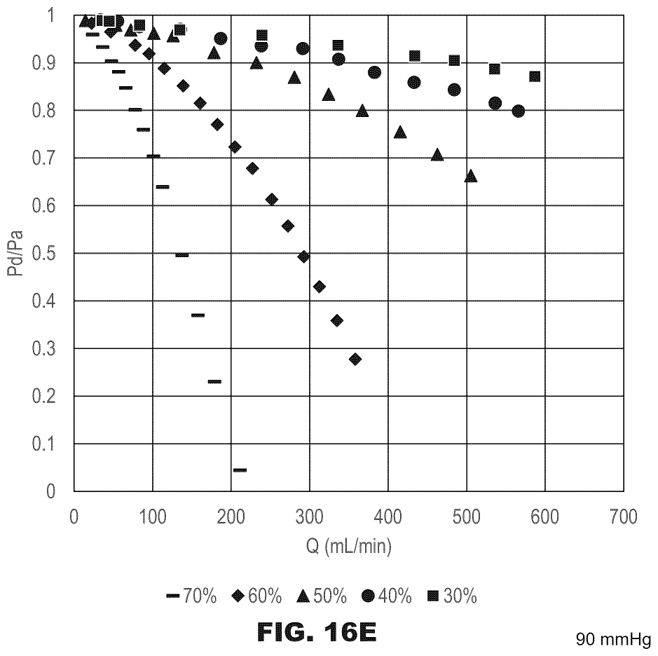

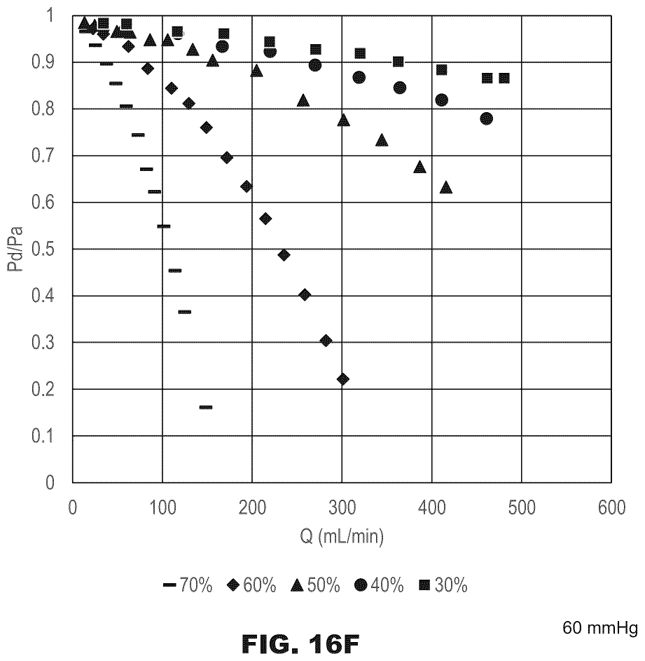

FIGS. 16A-C illustrate pressure drops as a function of blood flow rates with various percent diameter stenosis. As shown in FIGS. 16A-C, there can be a quadratic relationship between pressure drop and blood flow rate. In FIGS. 16A-C, curves are shown under hypertensive (140 mmHg), normal (90 mmHg), and hypotensive (60 mmHg) conditions.

FIGS. 16D-F illustrate pressure ratios (e.g., Pd/Pa) at varying flow rates with various percent diameter stenoses. As shown in FIGS. 16D-F, there can be a quadratic relationship between Pd/Pa and blood flow rate. In FIGS. 16D-F, curves are shown under hypertensive (140 mmHg), normal (90 mmHg), and hypotensive (60 mmHg) conditions.

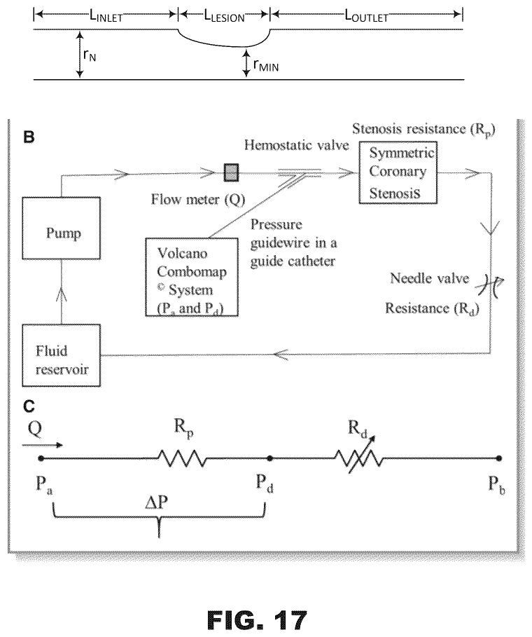

FIG. 17 is a diagram that schematically illustrates stenosis and fluid flow.

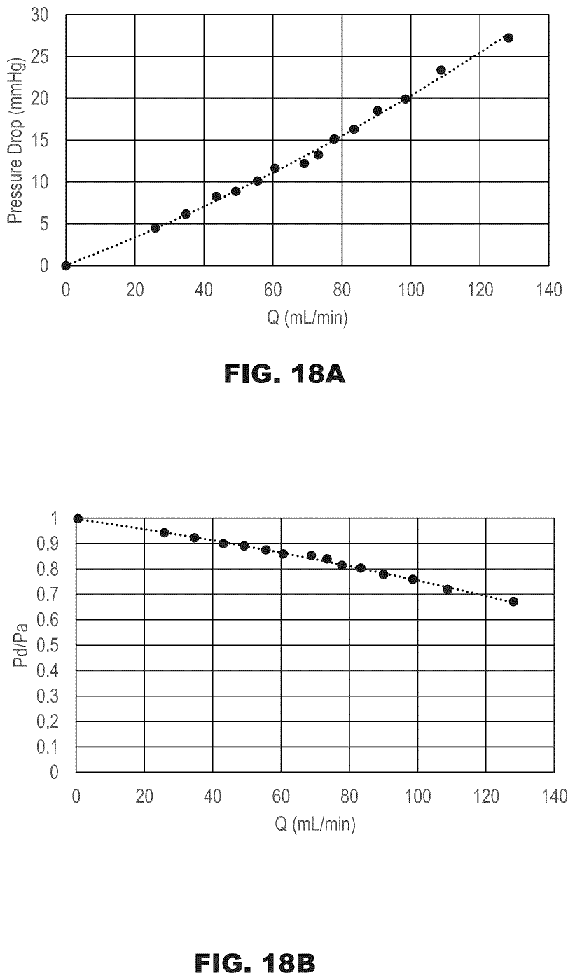

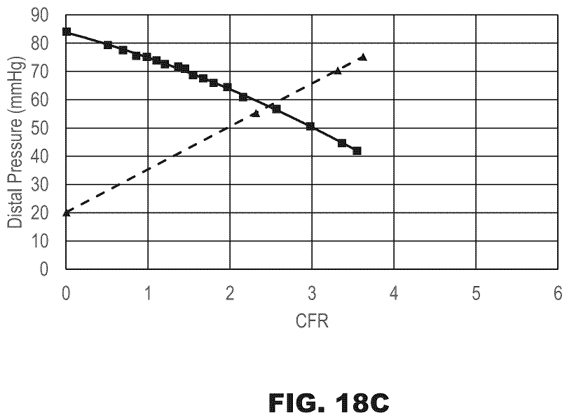

FIGS. 18A-18C illustrates graphs of pressure drop as a function of blood flow rate, pressure ratio as a function of blood flow rate, and distal pressure as a function of CFR. As shown in FIG. 18C, in some embodiments, FFR3D values can be determined based at least in part on the intersection of the two curves.

FIG. 18C is an example graph that shows coronary pressure as a function of coronary flow reserve. The CFR can be defined as the ratio of flow rate to a baseline flow rate. The baseline flow rate can be a resting flow rate. The dashed line relates the maximum possible flow for a particular coronary perfusion pressure under hyperemic conditions. The solid line is representative of the impact of a given stenosis. More specifically, the solid line represents pressure distal to the stenosis as a function of flow rate. The graph in FIG. 18C can be particular to specific proximal pressure and percent diameter stenosis. The dashed line indicates the relationship between coronary perfusion pressure and coronary flow reserve. The intersection of the two curves gives the distal pressure at hyperemia.

As described herein, FF3D can offer various advantages and features. For example, FFR 3D can be advantageous in that it is based upon actual measurements, is personalized for the patient, accounts for plaque (e.g., using materials with different properties for the atrial walls to represent soft plaque, calcified plaque, etc.), and so forth. FFR3D, as described herein, offers technical improvements and can be relatively inexpensive as compared with techniques such as invasive FFR. As discussed herein, the techniques in this disclosure are not limited to FFR values, but can, additionally or alternatively, be used to determine prescribed flow reserve, iFR, HSR, MBF/CFR, CFR-FFR mismatch, and/or wall shear stress.

As described herein, idealized stenoses models have a number of limitations. Idealized stenoses can be used to provide a simplified model of narrowing in a blood vessel. However, there can be many limitations associated with the use of idealized stenoses. For example, idealized stenoses typically have some symmetry (e.g., symmetric about the central axis of the blood vessel), have smooth contours, and so forth. These idealized stenoses can deviate significantly from real stenoses, which can be irregular, of varying lengths, be composed of different amounts and/or types of plaque, and so forth. In some cases, idealized modeling may include only one stenosis, but in real patients there can be multiple stenoses that can each contribute to ischemia. Additionally, stenoses in one vessel can influence ischemia in another vessel.

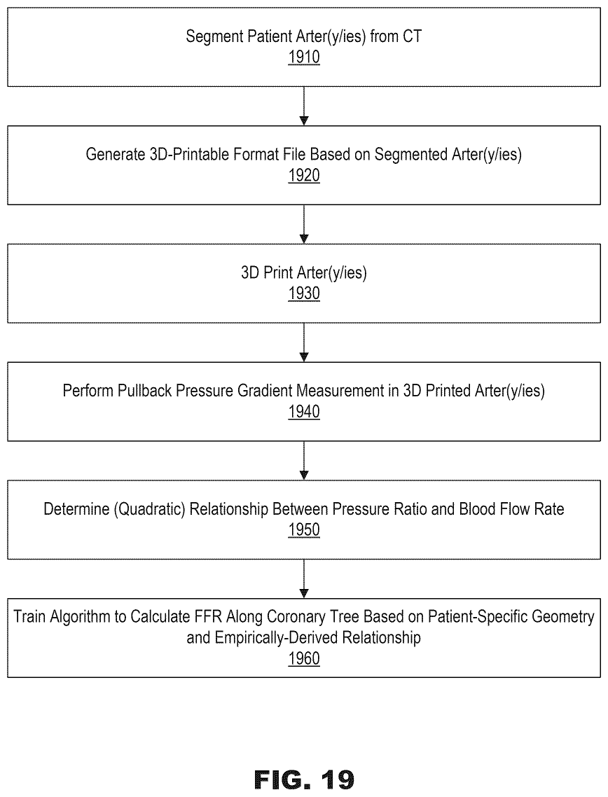

FIG. 19 is a flowchart that illustrates an example process for training an algorithm to predict FFR values along a coronary tree according to some embodiments. While described with respect to a coronary tree, it will be appreciated that the process illustrated in FIG. 19 can be applied to a portion of the coronary tree or to other vessels in the body. At operation 1910, a system can segment patient arteries from a CT image (e.g., a CCTA image). For example, the system can use a first machine learning algorithm configured to access a CT image and segment vessels in the image. At operation 1920, the system can generate a 3D-printable file (e.g., an STL file) based on the segmented arteries. At operation 1930, the system can 3D print the arteries. For example, the system can include or can be in communication with a suitable 3D printer and can cause the 3D printer to print the arteries (e.g., using the STL file). At operation 1940, a user can perform pullback pressure gradient measurements in the 3D-printed arteries and the system can receive the pullback pressure gradient measurements, for example either automatically or via user input of measurement values.

At operation 1950, the system can determine a relationship (e.g., a quadratic relationship) between a pressure ratio (e.g., Pd/Pa) and a fluid flow rate. In some implementations, the fluid can be a fluid that mimics the fluid properties of blood (e.g., the fluid can be a non-Newtonian fluid). A blood-mimicking fluid can be any fluid with suitable flow properties, such as a mixture of water and glycerol, for example a 3:2 mixture by volume of distilled water and glycerol. At operation 1960, the system can train an algorithm (e.g., a second machine learning algorithm) to calculate FFR values along a coronary tree based on patient-specific geometry and an empirically derived relationship (e.g., the relationship derived at operation 1950 between pressure and fluid flow rate).

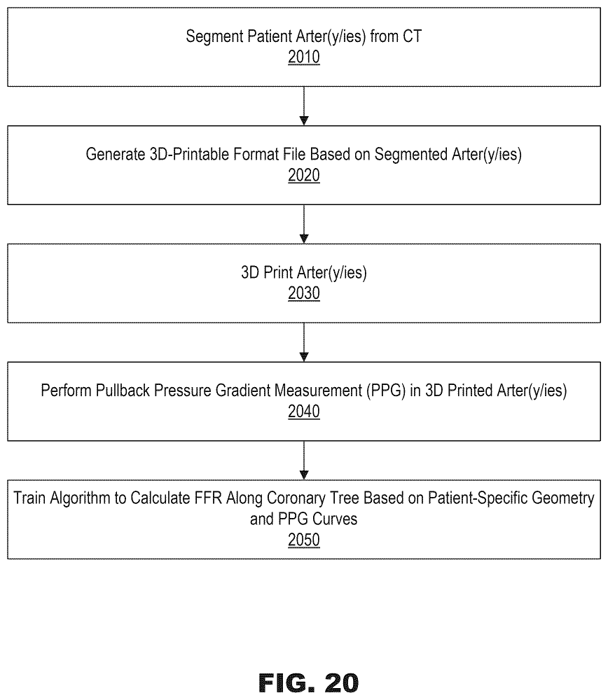

FIG. 20 is a flowchart the illustrates an example process for training an algorithm to predict FFR based on patient-specific geometry and pressure pullback gradient (PPG) curves according to some embodiments. While described in the context of multiple arteries and a coronary tree, it will be appreciated that the approach described in FIG. 20 can be readily applied to any vessel or to multiple vessels. At operation 2010, a system can segment patient arteries from a CT image (e.g., a CCTA image). At operation 2020, the system can generate a 3D-printable file based on the segmented arteries, such as an STL file. At operation 2030, the system can 3D print the arteries, for example by communicating with a 3D printer. At operation 1740, a user can PPG measurements in the 3D printed arteries, and the system can receive the PPG measurements, for example either automatically or via user input of measurement values. At operation 2050, the system can train an algorithm to calculate FFR values along a coronary tree based on patient-specific geometry (e.g., as determined from the CT image) and the PPG curves.

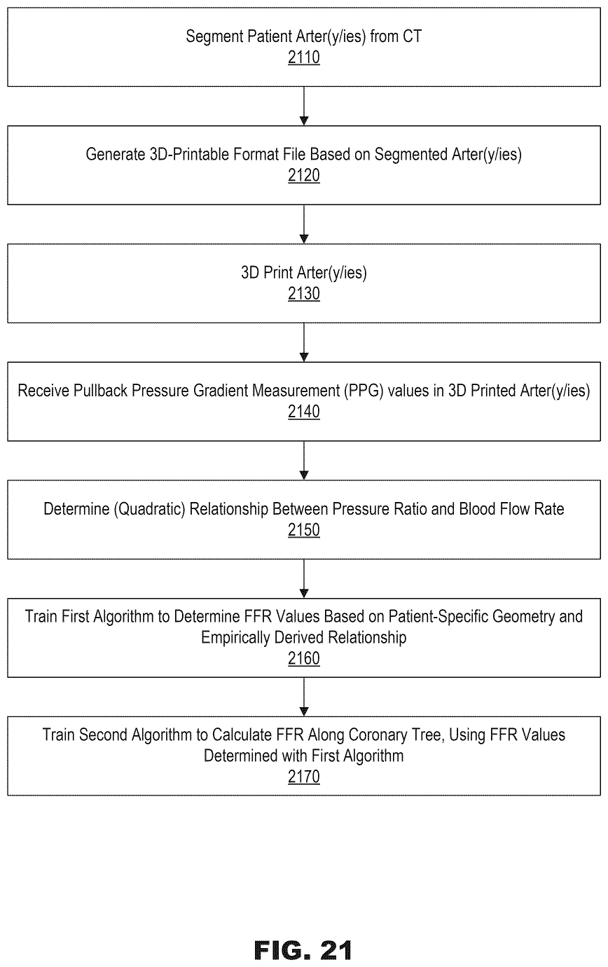

FIG. 21 is a flowchart that illustrates an example multi-algorithm process according to some embodiments. It will be appreciated that the process shown in FIG. 21 is not strictly limited to coronary arteries. Several operations in FIG. 21 are broadly similar to those in, for example, FIGS. 19 and 20, and are discussed only briefly in the following description. At operation 2110, a system can segment patient arteries from a CT image. At operation 2120, the system can generate a 3D-printable file (e.g., STL file) based on the segmented arteries. At operation 2130, the system can 3D print the arteries. At operation 2140, the system can receive PPG values measured in the 3D-printed arteries. At operation 2150, the system can determine a quadratic relationship between PPG and blood flow rate. At operation 2160, the system can train a first algorithm for predicting FFR values based on patient-specific geometry and the relationship determined at operation 2150. At operation 2170, the system can train a second algorithm to calculate FFR along a coronary tree using FFR values determined with the first algorithm.

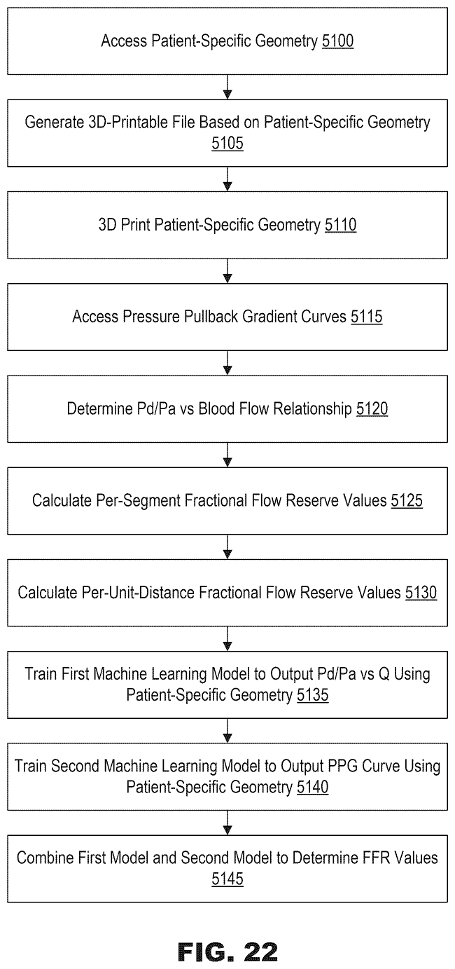

FIG. 22 is a drawing that illustrates an example process for developing an algorithm for FFR estimation/calculation using patient-specific geometry/anatomic inputs to estimate quadratic relationships and PPG curves according to some embodiments. At operation 5100, a system can access patient-specific geometry that describes or depicts one or more vessels, such as coronary vessels. The patient-specific geometry can be captured in a CCTA image or set of CCTA images. In some embodiments, a machine learning algorithm is used to extract vessels (e.g., coronary vessels) from one or more CCTA images. At operation 5105, the system can generate a 3D-printable file based on the patient-specific geometry. At operation 5110, the system can 3D print the patient-specific geometry using the 3D-printable file. For example, the system can instruct a 3D printer to print the patient-specific geometry. In some embodiments, only a portion of the patient-specific geometry, such as a specific vessel, is 3D printed. At operation 5115, the system can access one or more pressure pullback gradient curves. The pressure pullback gradient curves can be generated by accessing pressure measurements collected by flowing a fluid through the 3D-printed vessels and sensing pressure within the vessels. At operation 5120, the system can analyze the pressure data, along with relevant parameters such as inlet pressure, to determine a ratio of distal and proximal pressure (Pd/Pa) as a function of flow rate Q. As described herein, the ratio Pd/Pa vs. Q can be used to determine FFR values. At operation 5125, the system can calculate per-segment FFR values. At operation 5130, the system can determine per-unit-distance FFR values. In some embodiments, FFR values may be determined at any combination of one or more of a segment level, unit distance level, or vessel level. At operation 5135, the system can train a first machine learning model to output Pd/Pa vs. Q using patient-specific geometry as inputs. For example, operations 5100-5130 can be performed for a plurality of subjects, vessels, or both, and the resulting information can be used for training the first machine learning model. Similarly, at operation 5140, the system can train a second machine learning model to output PPG curves using patient-specific geometry. The second machine learning model can similarly be trained using data collected and analyzed for a plurality of subjects, vessels, or both. At operation 5145, the system can combine the first machine learning model and second machine learning model to produce a final model that can be used for determined FFR values. For example, for a new patient, the new patient's vessel structure can be extracted from one or more images (e.g., CCTA) images, and this information can be provided to the first machine learning model and the second machine learning model. Both the first and second machine learning models can output FFR values. Final FFR values can be, for example, an average from the first machine learning model and the second machine learning model. In some embodiments, FFR values can be identified as valid or invalid (or an indication of confidence can be otherwise provided) based on a degree of mismatch between outputs from the first machine learning model and the second machine learning model. FIG. 22 shows two independent machine learning models. However, it will be appreciated that the machine learning models are not necessarily independent. For example, in some embodiments, outputs of the first machine learning model are used as inputs for training the second machine learning model, or outputs from the second machine learning model are used as inputs for training the first machine learning model. In such embodiments, new patient geometry information is run through one model, then the other (which uses outputs of the one model as inputs to the other model), rather than, for example, parallel application of the first machine learning model and the second machine learning model.

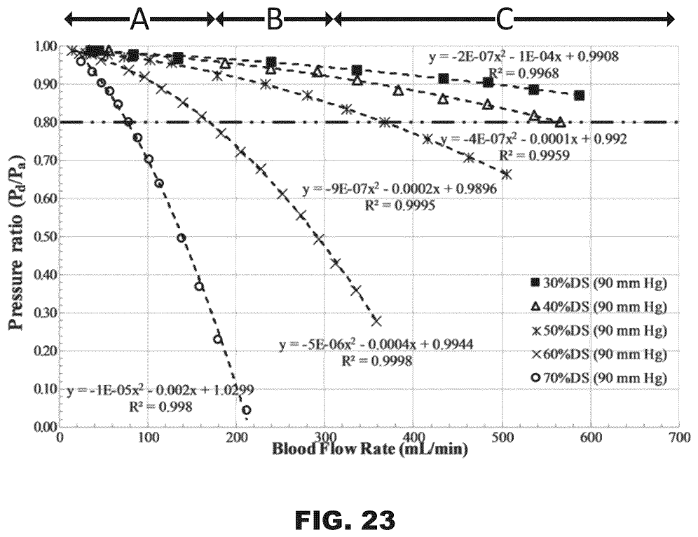

FIG. 23 is a plot that illustrates prescribed flow reserve concepts according to some embodiments. As described herein, FFR measurements, which relate to hyperemic states, may not be reflective of a patient's regular activities of daily living. For example, a patient who does not engagement in vigorous exercise may rarely or never achieve blood flow rates in the zone labeled “C.” For example, a patient who only goes for casual walks or whose physical activities are limited to, for example, grocery shopping or getting the mail may only regularly achieve flow rates in the zone labeled “B.” Prescribed flow reserve can be used to investigate the impact of stenoses in the context of a patient's activities of daily living.

As described herein, computational fluid dynamics (CFD), while a powerful tool, can have many drawbacks. The computational demands to perform high quality, accurate CFD calculations can be very computationally expensive. To make calculations computationally feasible, many assumptions and simplifications are often made, which can lead to inaccurate results, especially for vessels with severe stenoses or other issues that result in significant deviations from idealized behavior. However, CFD calculations can be useful for generating initial training data for a machine learning algorithm. The algorithm can be fine-tuned using data obtained from 3D printed arteries.

Zero dimensional CFD (OD CFD) can be used to model fluid flow in vessels. In OD CFD, spatial information is absent, and flow is purely time dependent. That is, OD CFD calculations can provide global information about flow, but not spatial information. OD CFD can be used to simulate bulk flow rates, pressures, etc., within a vessel. In OD CFD, a fluidic system can be modeled similarly to an electronic circuit. For example, resistors can represent viscosity or stenoses, capacitors can represent the compliance of a vessel wall, inductors can represent inertia of fluid flow, and so forth. In some implementations, multiple circuit elements can be combined to define a stenosis element. In some implementations, the stenosis element can be used to model the energy dissipation in a fluid caused by stenosis.

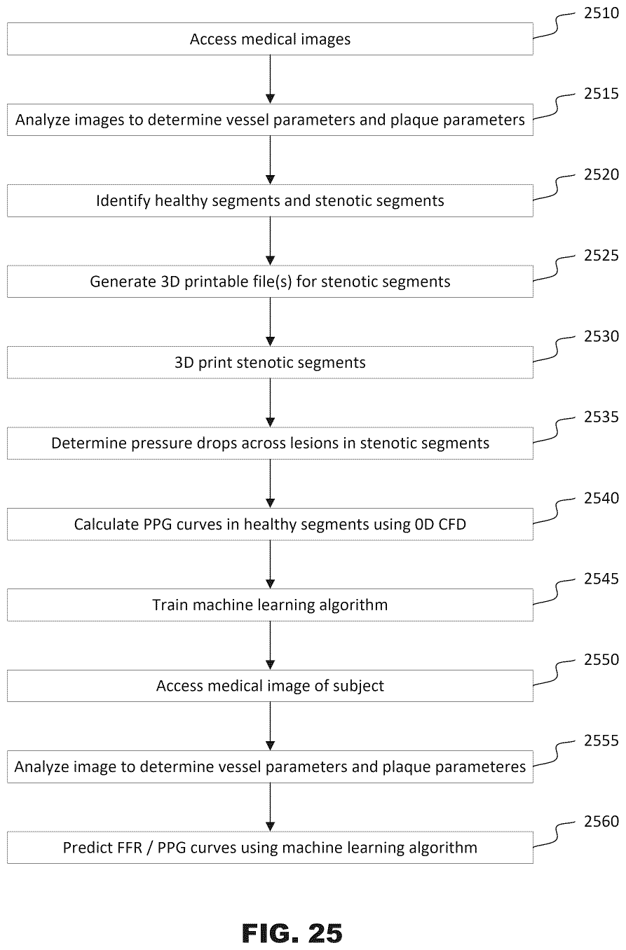

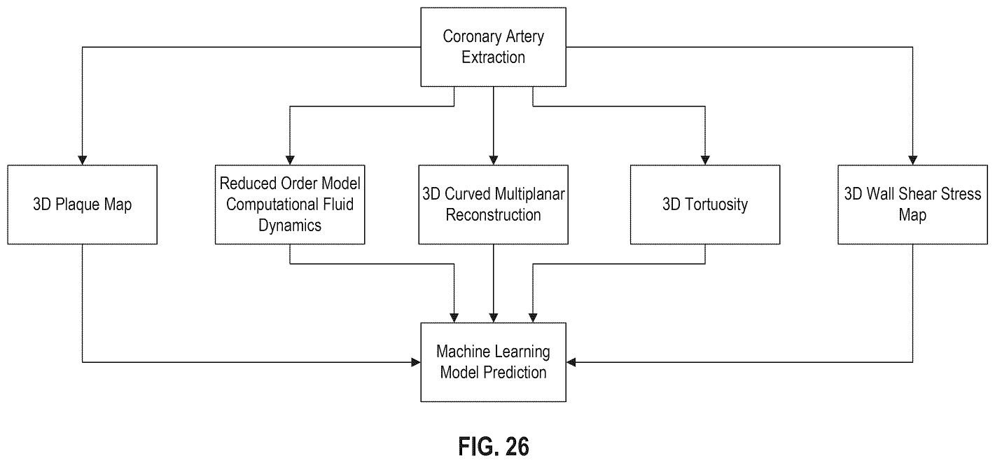

OD CFD calculations can perform relatively well (e.g., can closely represent fluid flow in actual vessels) for simple scenarios, such as locations within vessels that have relatively constant luminal diameters, but may perform poorly when there is significant stenosis present in the vessel. In some implementations, OD CFD calculations are used as part of a training process, as discussed above and described in more detail below with respect to FIG. 26. In some embodiments, a combination of OD CFD and machine learning are used to model fluid flow properties in vessels, with OD CFD being used in healthy vessel regions and machine learning model(s) being used for more complex stenotic regions, for example as described below with respect to FIG. 25.

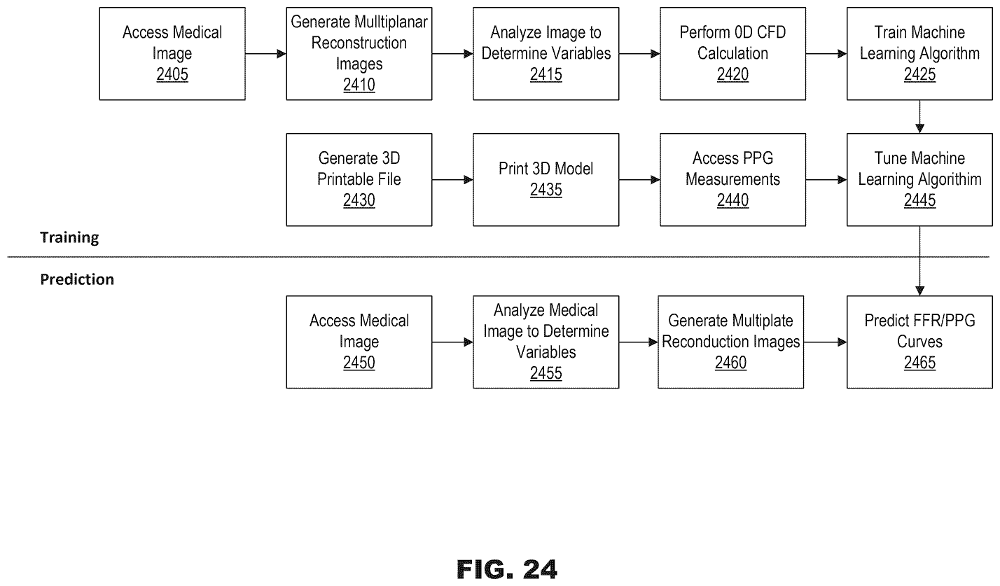

FIG. 24 is a flowchart that illustrates an example process for training and deploying a machine learning model according to some implementations.

At operation 2405, a system can access a medical image, for example a CCTA image. At operation 2410, the system can generate one or more reconstructions (e.g., straightened multiplanar reconstructions and/or curved multiplanar reconstructions). At operation 2415, the system can analyze the medical image to determine one or more variables, such as lumen diameter, plaque volume, plaque length, percent diameter stenosis, and so forth. At operation 2420, the system can perform a OD CFD calculation based on the determined variables. Operations 2405-2020 can be performed for a plurality of medical images to produce a training data set. At operation 2425, the system can train a machine learning algorithm based on the OD CFD calculations and the determined variables and/or the reconstructed images. For example, the machine learning model can take the variables and/or reconstructed images as input and can be trained using supervised learning to reproduce the outputs of the OD CFD calculations and/or values derived from the OD CFD calculations (e.g., FFR values). At operation 2430, the system can generate a 3D printable file (e.g., an STL file) of the arteries. At operation 2435, the system can cause the file to be printed to generate a physical 3D model. At operation 2440, the system can access pressure pullback gradient (PPG) measurements captured by measuring flow properties within the physical 3D model. For example, a user can input values or values can be collected automatically by the system, for example from a pressure sensor in communication with the system. At operation 2445, the system can tune the machine learning algorithm based on the PPG measurements and the known or measured geometry of the vessels in the physical 3D model.

After training, the machine learning algorithm can be deployed for use on new medical images. At operation 2450, the system (which can be the same system or a different system from the one used for training) can access a medical image of a subject. At operation 2455, the system can analyze the medical image to determine one or more parameters. At operation 2460, the system can generate one or more multiplanar reconstructions based on the medical image of the subject. At operation 2465, the system can, using the machine learning algorithm, predict fractional flow reserve parameters, pressure pullback gradient parameters (e.g., PPG curves), or both. In some embodiments, multiplanar reconstructions are not generated. For example, in some embodiments, values to be input into the machine learning model (e.g., lumen diameter, stenosis area/length/thickness, etc., can be derived from a medical image without multiplanar reconstruction).

In FIG. 24, the process includes both multiplanar reconstruction images and vessel/plaque parameters. In some implementations, both are used to train the algorithm and/or as inputs to the algorithm. However, it is not necessary to use both. In some implementations, only multiplanar reconstruction images (e.g., curved multiplanar reconstructions (CM PRs) or straightened multiplanar reconstructions (SM PRs)) or only determined parameters are used in training and/or deploying the machine learning algorithm. Thus, in some implementations, the system may generate only one of multiplanar reconstruction images or vessel/plaque parameters, or the system may generate both but only use one as inputs to the machine learning model. For example, vessel and/or plaque parameters can be determined from multiplanar reconstructions and input into the machine learning model.

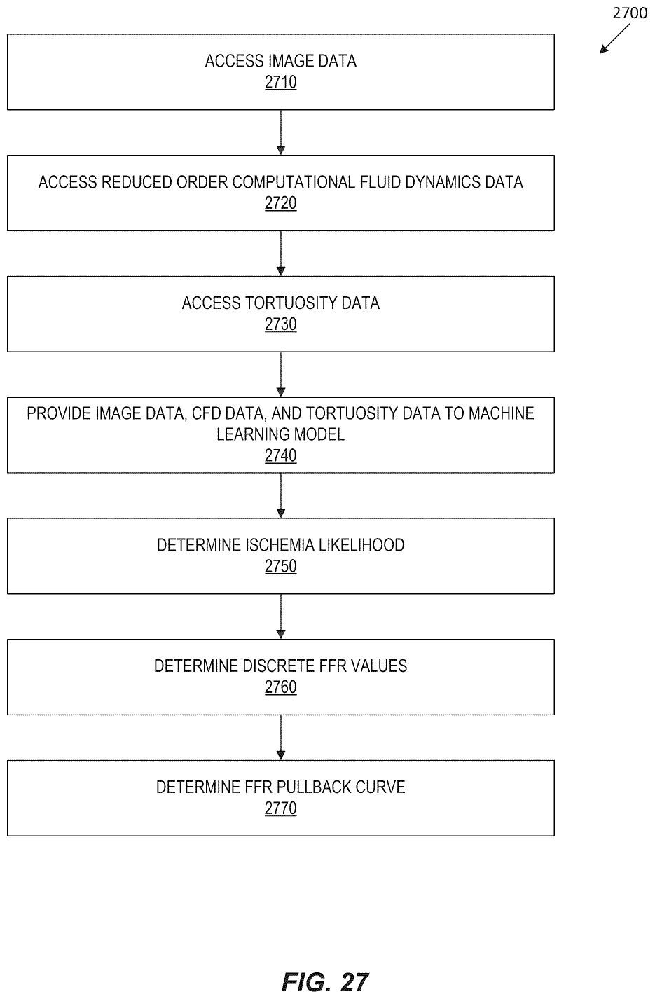

FIG. 25 is a flowchart that illustrates an example process for combining OD CFD calculations and 3D printing according to some implementations. In FIG. 27, OD CFD calculations are used in healthy segments of vessels, while a machine learning algorithm is used in stenotic segments of vessels. The machine learning model can be trained in various manners, for example, as described herein, and can be trained using OD CFD calculations or not (e.g., using only OD CFD calculations, using OD CFD calculations and measurements collected from 3D-printed vessels, using only measurements collected from 3D-printed vessels, or, in some embodiments, using invasive FFR measurements from actual patients), or any combination thereof).

At operation 2510, a system can access one or more medical images, for example, CCTA images. At operation 2515, the system can analyze the medical images to determine vessel parameters and plaque parameters. At operation 2520, the system can identify healthy segments and stenotic segments of the vessels. At operation 2525, the system can generate 3D printable files (e.g., STL files) of the stenotic segments. At operation 2530, the system can cause a 3D printer to print physical representations of the stenotic segments. At operation 2535, the system can determine pressure drops across lesions in the stenotic segments. For example, a technique can collect PPG data by pulling a pressure-sensitive catheter through the stenotic segments while a fluid flows through the stenotic segments. At operation 2540, the system can calculate PPG curves in healthy segments using OD CFD. At operation 2545, the system can train a machine learning algorithm using the data collected from the 3D printed stenotic segments and the OD CFD calculations. For example, the determined plaque parameters and vessel parameters can be provided as inputs and the machine learning algorithm can be trained using supervised learning to produce the values determined from OD CFD calculations and the data obtained from the 3D-printed stenotic segments.

At operation 2550, the system can access a medical image of a subject, for example, a CCTA image. At operation 2555, the system can analyze the image to determine vessel parameters and plaque parameters. At operation 2560, the system can use the machine learning algorithm to predict FFR values, FFR curves, PPG values, etc.

### Example Non-ML FFR3D Embodiments

Embodiment 1. A computer-implemented method for estimating fractional flow reserve for a subject based on image analysis of a medical image of the subject, the computer-implemented method comprising: accessing, by a computer system, the medical image of the subject, wherein the medical image of the subject depicts one or more regions of one or more coronary arteries of the subject; analyzing, by the computer system, the medical image of the subject to identify the one or more regions of the one or more coronary arteries; determining, by the computer system, vessel geometry of the identified one or more regions of the one or more coronary arteries, wherein the vessel geometry is determined based at least in part on a lumen wall and a vessel wall of the identified one or more regions of the one or more coronary arteries; determining, by the computer system, a fluid flow characteristic of the identified one or more regions of the one or more coronary arteries based on the determined vessel geometry and predetermined relationships between vessel geometry and fluid flow determined using a plurality of three-dimensional (3D) printed models of coronary arteries of a plurality of sample subjects, wherein each of the predetermined relationships comprises a relationship between pressure and fluid flow rate, wherein the predetermined relationships are determined by: for each sample medical image of a plurality of sample medical images associated with a plurality of sample subjects: accessing the sample medical image; analyzing the sample medical image to identify a plurality of vessels depicted in the sample medical image; determining a geometry of each vessel of the plurality of vessels; generating a 3D-printable model of the plurality of vessels, wherein the 3D-printable model comprises a file stored in a non-transitory computer-readable medium; accessing pressure measurements collected from a physical 3D model of the plurality of vessels produced by a 3D printer using the 3D-printable model, wherein the pressure measurements are collected by positioning a pressure sensor in a lumen of the physical 3D model at a plurality of locations within the physical 3D model, wherein the pressure measurements are collected at a plurality of inlet pressures, and wherein the pressure measurements are collected at a plurality of fluid flow rates; determining, by the computer system using the determined fluid flow characteristic of the identified one or more regions of the one or more coronary arteries, one or more fractional flow reserve values associated with one or more locations within the one or more regions of the one or more coronary arteries of the subject, wherein the computer system comprises a computer processor and an electronic storage medium.
Embodiment 2. A computer-implemented method for estimating fractional flow reserve for a subject based on image analysis of a medical image of the subject, the computer-implemented method comprising: accessing, by a computer system, the medical image of the subject, wherein the medical image of the subject depicts one or more regions of one or more coronary arteries of the subject; analyzing, by the computer system, the medical image of the subject to identify the one or more regions of the one or more coronary arteries; determining, by the computer system, vessel geometry of the identified one or more regions of the one or more coronary arteries, wherein the vessel geometry is determined based at least in part on a lumen wall and a vessel wall of the identified one or more regions of the one or more coronary arteries; determining, by the computer system, a fluid flow characteristic of the identified one or more regions of the one or more coronary arteries based on the determined vessel geometry and predetermined relationships between vessel geometry and fluid flow determined using a plurality of three-dimensional (3D) printed models of coronary arteries of a plurality of sample subjects; determining, by the computer system using the determined fluid flow characteristic of the identified one or more regions of the one or more coronary arteries, one or more fractional flow reserve values associated with one or more locations within the one or more regions of the one or more coronary arteries of the subject, wherein the computer system comprises a computer processor and an electronic storage medium.
Embodiment 3. The computer-implemented method of embodiment 2, wherein the predetermined relationships are determined by: for each sample medical image of a plurality of sample medical images associated with a plurality of sample subjects: accessing the sample medical image; analyzing the sample medical image to identify a plurality of vessels depicted in the sample medical image; determining a geometry of each vessel of the plurality of vessels; generating a 3D-printable model of the plurality of vessels, wherein the 3D-printable model comprises a file stored in a non-transitory computer-readable medium; accessing pressure measurements collected from a physical 3D model of the plurality of vessels produced by a 3D printer using the 3D-printable model, wherein the pressure measurements are collected by positioning a pressure sensor in a lumen of the physical 3D model at a plurality of locations within the physical 3D model, wherein the pressure measurements are collected at a plurality of inlet pressures, and wherein the pressure measurements are collected at a plurality of fluid flow rates.
Embodiment 4. The computer-implemented method of embodiment 3, wherein the predetermined relationship is a relationship between pressure and fluid flow rate.
Embodiment 5. The computer-implemented method of embodiment 3 or 4, wherein the medical image is a coronary computed tomography angiography image.
Embodiment 6. The computer-implemented method of any of embodiments 3-5, wherein the wherein the one or more coronary arteries comprise one or more of left main (LM), ramus intermedius (RI), left anterior descending (LAD), diagonal one (D1), diagonal two (D2), left circumflex (Cx), obtuse marginal one (OM1), obtuse marginal two (OM2), left posterior descending (L-PDA), left posterolateral branch (L-PLB), right coronary (RCA), right posterior descending (R-PDA), or right posterolateral branch (R-PLB).
Embodiment 7. The computer-implemented method of embodiment 4 or 5, wherein at least one physical 3D model is printed using a plurality of materials, wherein at least one material of the plurality of materials is selected based on a density of a region of plaque identified in the sample medical image.
Embodiment 8. The computer-implemented method of embodiment 7, wherein the density of the region of plaque is determined based on a radiodensity of the region of plaque in the sample medical image, wherein the density is at least one of: low density non-calcified plaque, non-calcified plaque, or calcified plaque.
Embodiment 9. The computer-implemented method of embodiment 8, wherein low density non-calcified plaque corresponds to a radiodensity of between about −189 Hounsfield units (HU) and about 30 HU, wherein non-calcified plaque corresponds to a radiodensity of between about 31 HU and about 350 HU, and wherein calcified plaque corresponds to a radiodensity of between about 351 HU and about 2500 HU.
Embodiment 10. The computer-implemented method of any of embodiments 3-9, wherein a fractional flow reserve value of the one or more fractional flow reserve values is determined as a ratio of pressure distal to a region of plaque and pressure proximal to the region of plaque.
Embodiment 11. The computer-implemented method of any of embodiments 3-10, wherein the one or more fractional flow reserve values is determined for at least one of: a vessel, a vessel segment, or a vessel unit length.
Embodiment 12. A system for estimating fractional flow reserve for a subject based on image analysis of a medical image of the subject, the system comprising: at least one processor; and a computer-readable medium storing instructions that, when executed by the system, cause the system to: access the medical image of the subject, wherein the medical image of the subject depicts one or more regions of one or more coronary arteries of the subject; analyze the medical image of the subject to identify the one or more regions of the one or more coronary arteries; determine vessel geometry of the identified one or more regions of the one or more coronary arteries, wherein the vessel geometry is determined based at least in part on a lumen wall and a vessel wall of the identified one or more regions of the one or more coronary arteries; determine a fluid flow characteristic of the identified one or more regions of the one or more coronary arteries based on the determined vessel geometry and predetermined relationships between vessel geometry and fluid flow determined using a plurality of three-dimensional (3D) printed models or coronary arteries of a plurality of sample subjects; determine, using the determined fluid flow characteristic of the identified one or more regions of the one or more coronary arteries, one or more fractional flow reserve values associated with one or more locations within the one or more regions of the one or more coronary arteries of the subject.
Embodiment 13. The system of embodiment 12, wherein the predetermined relationships are determined by: for each sample medical image of a plurality of sample medical images associated with a plurality of sample subjects: accessing the sample medical image; analyzing the sample medical image to identify a plurality of vessels depicted in the sample medical image; determining a geometry of each vessel of the plurality of vessels; generating a 3D-printable model of the plurality of vessels, wherein the 3D-printable model comprises a file stored in a non-transitory computer-readable medium; accessing pressure measurements collected from a physical 3D model of the plurality of vessels produced by a 3D printer using the 3D-printable model, wherein the pressure measurements are collected by positioning a pressure sensor in a lumen of the physical 3D model at a plurality of locations within the physical 3D model, wherein the pressure measurements are collected at a plurality of inlet pressures, and wherein the pressure measurements are collected at a plurality of fluid flow rates.
Embodiment 14. The system of embodiment 13, wherein the predetermined relationship is a relationship between pressure and fluid flow rate.
Embodiment 15. The system of embodiment 13 or 14, wherein the medical image is a coronary computed tomography angiography image.
Embodiment 16. The system of embodiment 13, 14, or 15, wherein the wherein the one or more coronary arteries comprise one or more of left main (LM), ramus intermedius (RI), left anterior descending (LAD), diagonal one (D1), diagonal two (D2), left circumflex (Cx), obtuse marginal one (OM1), obtuse marginal two (OM2), left posterior descending (L-PDA), left posterolateral branch (L-PLB), right coronary (RCA), right posterior descending (R-PDA), or right posterolateral branch (R-PLB).
Embodiment 17. The system of embodiment 14, wherein at least one physical 3D model is printed using a plurality of materials, wherein at least one material of the plurality of materials is selected based on a density of a region of plaque identified in the sample medical image.
Embodiment 18. The system of embodiment 17, wherein the density of the region of plaque is determined based on a radiodensity of the region of plaque in the sample medical image, wherein the density is at least one of: low density non-calcified plaque, non-calcified plaque, or calcified plaque.
Embodiment 19. The system of embodiment 18, wherein low density non-calcified plaque corresponds to a radiodensity of between about −189 Hounsfield units (HU) and about 30 HU, wherein non-calcified plaque corresponds to a radiodensity of between about 31 HU and about 350 HU, and wherein calcified plaque corresponds to a radiodensity of between about 351 HU and about 2500 HU.
Embodiment 20. The system of any of embodiments 13-19, wherein a fractional flow reserve value of the one or more fractional flow reserve values is determined as a ratio of pressure distal to a region of plaque and pressure proximal to the region of plaque.

### Example ML FFR3D Embodiments

Embodiment 1. A computer-implemented method for estimating fractional flow reserve for a subject based on image analysis of a medical image of the subject, the computer-implemented method comprising: accessing, by a computer system, the medical image of the subject, wherein the medical image of the subject depicts one or more regions of one or more coronary arteries of the subject; analyzing, by the computer system, the medical image of the subject to identify the one or more regions of the one or more coronary arteries; determining, by the computer system, vessel geometry of the identified one or more regions of the one or more coronary arteries, wherein the vessel geometry is determined based at least in part on a lumen wall and a vessel wall of the identified one or more regions of the one or more coronary arteries; generating an input for a machine learning model for determining a fluid flow characteristic, the input based at least in part on the determined vessel geometry; determining, by the computer system using the machine learning model, the fluid flow characteristic of the identified one or more regions of the one or more coronary arteries, wherein the machine learning model is trained to determine the fluid flow characteristic using fluid flow measurements collected from a plurality of three-dimensional (3D) printed models of coronary arteries of a plurality of sample subjects, and wherein the machine learning model is trained to determine the fluid flow characteristic by: for each sample medical image of a plurality of sample medical images associated with a plurality of sample subjects: accessing the sample medical image; analyzing the sample medical image to identify a plurality of vessels depicted in the sample medical image; determining a geometry of each vessel of the plurality of vessels; generating a 3D-printable model of the plurality of vessels, wherein the 3D-printable model comprises a file stored in a non-transitory computer-readable medium; and accessing pressure measurements collected from a physical 3D model of the plurality of vessels produced by a 3D printer using the 3D-printable model, wherein the pressure measurements are collected by positioning a pressure sensor in a lumen of the physical 3D model at a plurality of locations within the physical 3D model, wherein the pressure measurements are collected at a plurality of inlet pressures, and wherein the pressure measurements are collected at a plurality of fluid flow rates; and providing the determined geometries and the pressure measurements to the machine learning model, wherein the determined geometries are used as inputs, and wherein the determined pressure measurements are used as outputs, wherein the machine learning model is trained to output a pressure pullback gradient curve; determining, by the computer system using the determined fluid flow characteristic of the identified one or more regions of the one or more coronary arteries, one or more fractional flow reserve values associated with one or more locations within the one or more regions of the one or more coronary arteries of the subject, wherein the computer system comprises a computer processor and an electronic storage medium.
Embodiment 2. A computer-implemented method for estimating fractional flow reserve for a subject based on image analysis of a medical image of the subject, the computer-implemented method comprising: accessing, by a computer system, the medical image of the subject, wherein the medical image of the subject depicts one or more regions of one or more coronary arteries of the subject; analyzing, by the computer system, the medical image of the subject to identify the one or more regions of the one or more coronary arteries; determining, by the computer system, vessel geometry of the identified one or more regions of the one or more coronary arteries, wherein the vessel geometry is determined based at least in part on a lumen wall and a vessel wall of the identified one or more regions of the one or more coronary arteries; generating an input for a machine learning model for determining a fluid flow characteristic, the input based at least in part on the determined vessel geometry; determining, by the computer system using the machine learning model, the fluid flow characteristic of the identified one or more regions of the one or more coronary arteries, wherein the machine learning model is trained to determine the fluid flow characteristic using fluid flow measurements collected from a plurality of three-dimensional (3D) printed models of coronary arteries of a plurality of sample subjects; and determining, by the computer system using the determined fluid flow characteristic of the identified one or more regions of the one or more coronary arteries, one or more fractional flow reserve values associated with one or more locations within the one or more regions of the one or more coronary arteries of the subject, wherein the computer system comprises a computer processor and an electronic storage medium.
Embodiment 3. The computer-implemented method of embodiment 2, wherein the machine learning model is trained to determine the fluid flow characteristic by: for each sample medical image of a plurality of sample medical images associated with a plurality of sample subjects: accessing the sample medical image; analyzing the sample medical image to identify a plurality of vessels depicted in the sample medical image; determining a geometry of each vessel of the plurality of vessels; generating a 3D-printable model of the plurality of vessels, wherein the 3D-printable model comprises a file stored in a non-transitory computer-readable medium; and accessing pressure measurements collected from a physical 3D model of the plurality of vessels produced by a 3D printer using the 3D-printable model, wherein the pressure measurements are collected by positioning a pressure sensor in a lumen of the physical 3D model at a plurality of locations within the physical 3D model, wherein the pressure measurements are collected at a plurality of inlet pressures, and wherein the pressure measurements are collected at a plurality of fluid flow rates; and providing the determined geometries and the pressure measurements to the machine learning model, wherein the determined geometries are used as inputs and wherein the determined pressure measurements are used as outputs, wherein the machine learning model is trained to output the pressure measurements.
Embodiment 4. The computer-implemented method of embodiment 2 or 3, wherein the machine learning model is trained to output a pressure pullback gradient curve.
Embodiment 5. The computer-implemented method of any of embodiments 2-4, wherein the input includes information about a region of plaque detected in the one or regions of the one or more coronary arteries of the subject, wherein the information about the region of plaque includes one or more of: plaque length, plaque area, plaque volume, or plaque density.
Embodiment 6. The computer-implemented method of embodiment 5, wherein the plaque density is one or more of: low density non-calcified plaque, non-calcified plaque, or calcified plaque.
Embodiment 7. The computer-implemented method of embodiment 6, wherein the medical image is a coronary computed tomography angiography image, wherein low density non-calcified plaque corresponds to a radiodensity of between about −189 Hounsfield units (HU) and about 30 HU, wherein non-calcified plaque corresponds to a radiodensity of between about 31 HU and about 350 HU, and wherein calcified plaque corresponds to a radiodensity of between about 351 HU and about 2500 HU.
Embodiment 8. The computer-implemented method of any of embodiments 2-7, wherein the medical image is a coronary computed tomography angiography image.
Embodiment 9. The computer-implemented method of any of embodiments 2-8, wherein the wherein the one or more coronary arteries comprise one or more of left main (LM), ramus intermedius (RI), left anterior descending (LAD), diagonal one (D1), diagonal two (D2), left circumflex (Cx), obtuse marginal one (OM1), obtuse marginal two (OM2), left posterior descending (L-PDA), left posterolateral branch (L-PLB), right coronary (RCA), right posterior descending (R-PDA), or right posterolateral branch (R-PLB).
Embodiment 10. The computer-implemented method of embodiment 3, wherein at least one physical 3D model is printed using a plurality of materials, wherein at least one material of the plurality of materials is selected based on a density of a region of plaque identified in the sample medical image.
Embodiment 11. The computer-implemented method of embodiment 10, wherein the density of the region of plaque is determined based on a radiodensity of the region of plaque in the sample medical image, wherein the density is at least one of: low density non-calcified plaque, non-calcified plaque, or calcified plaque.
Embodiment 12. The computer-implemented method of claim any of embodiments 2-11, wherein a fractional flow reserve value of the one or more fractional flow reserve values is determined as a ratio of pressure distal to a region of plaque and pressure proximal to the region of plaque.
Embodiment 13. The computer-implemented method of any of embodiments 2-12, wherein the one or more fractional flow reserve values is determined for at least one of: a vessel, a vessel segment, or a vessel unit length.
Embodiment 14. A system for estimating fractional flow reserve for a subject based on image analysis of a medical image of the subject, the system comprising: at least one processor; and a computer-readable medium storing instructions that, when executed by the system, cause the system to: access the medical image of the subject, wherein the medical image of the subject depicts one or more regions of one or more coronary arteries of the subject; analyze the medical image of the subject to identify the one or more regions of the one or more coronary arteries; determine vessel geometry of the identified one or more regions of the one or more coronary arteries, wherein the vessel geometry is determined based at least in part on a lumen wall and a vessel wall of the identified one or more regions of the one or more coronary arteries; generate an input for a machine learning model for determining a fluid flow characteristic, the input based at least in part on the determined vessel geometry; determine, using the machine learning model, the fluid flow characteristic of the identified one or more regions of the one or more coronary arteries, wherein the machine learning model is trained to determine the fluid flow characteristic using fluid flow measurements collected from a plurality of three-dimensional (3D) printed models of coronary arteries of a plurality of sample subjects; and determine, using the determined fluid flow characteristic of the identified one or more regions of the one or more coronary arteries, one or more fractional flow reserve values associated with one or more locations within the one or more regions of the one or more coronary arteries of the subject.
Embodiment 15. The system of embodiment 14, wherein the machine learning model is trained to determine the fluid flow characteristic by: for each sample medical image of a plurality of sample medical images associated with a plurality of sample subjects: accessing the sample medical image; analyzing the sample medical image to identify a plurality of vessels depicted in the sample medical image; determining a geometry of each vessel of the plurality of vessels; generating a 3D-printable model of the plurality of vessels, wherein the 3D-printable model comprises a file stored in a non-transitory computer-readable medium; and accessing pressure measurements collected from a physical 3D model of the plurality of vessels produced by a 3D printer using the 3D-printable model, wherein the pressure measurements are collected by positioning a pressure sensor in a lumen of the physical 3D model at a plurality of locations within the physical 3D model, wherein the pressure measurements are collected at a plurality of inlet pressures, and wherein the pressure measurements are collected at a plurality of fluid flow rates; providing the determined geometries and the pressure measurements to the machine learning model, wherein the determined geometries are used as inputs and wherein the determined pressure measurements are used as outputs, wherein the machine learning model is trained to output the pressure measurements.
Embodiment 16. The system of embodiment 14 or 15, wherein the machine learning model is trained to output a pressure pullback gradient curve.
Embodiment 17. The system of any of embodiments 14-16, wherein the input includes information about a region of plaque detected in the one or regions of the one or more coronary arteries of the subject, wherein the information about the region of plaque includes one or more of: plaque length, plaque area, plaque volume, or plaque density.
Embodiment 18. The system of embodiment 17, wherein the plaque density is one or more of: low density non-calcified plaque, non-calcified plaque, or calcified plaque.
Embodiment 19. The system of embodiment 18, wherein the medical image is a coronary computed tomography angiography image, wherein low density non-calcified plaque corresponds to a radiodensity of between about −189 Hounsfield units (HU) and about 30 HU, wherein non-calcified plaque corresponds to a radiodensity of between about 31 HU and about 350 HU, and wherein calcified plaque corresponds to a radiodensity of between about 351 HU and about 2500 HU.
Embodiment 20. The system of claim any of embodiments 14-19, wherein the medical image is a coronary computed tomography angiography image.
Embodiment 21. The system of claim any of embodiments 14-20, wherein the wherein the one or more coronary arteries comprise one or more of left main (LM), ramus intermedius (RI), left anterior descending (LA D), diagonal one (D1), diagonal two (D2), left circumflex (Cx), obtuse marginal one (OM1), obtuse marginal two (OM2), left posterior descending (L-PDA), left posterolateral branch (L-PLB), right coronary (RCA), right posterior descending (R-PDA), or right posterolateral branch (R-PLB).
Embodiment 22. The system of embodiment 15, wherein at least one physical 3D model is printed using a plurality of materials, wherein at least one material of the plurality of materials is selected based on a density of a region of plaque identified in the sample medical image.
Embodiment 23. The system of embodiment 22, wherein the density of the region of plaque is determined based on a radiodensity of the region of plaque in the sample medical image, wherein the density is at least one of: low density non-calcified plaque, non-calcified plaque, or calcified plaque.
Embodiment 24. The system of claim any of embodiments 14-23, wherein a fractional flow reserve value of the one or more fractional flow reserve values is determined as a ratio of pressure distal to a region of plaque and pressure proximal to the region of plaque.
Embodiment 25. The system of any of embodiments 14-24, wherein the one or more fractional flow reserve values is determined for at least one of: a vessel, a vessel segment, or a vessel unit length. Summary

### Example Combined Non-ML and ML FFR 3D Embodiments

Embodiment 1. A computer-implemented method for estimating fractional flow reserve for a subject based on image analysis of a medical image of the subject, the computer-implemented method comprising: accessing, by a computer system, the medical image of the subject, wherein the medical image of the subject depicts one or more regions of one or more coronary arteries of the subject; analyzing, by the computer system, the medical image of the subject to identify the one or more regions of the one or more coronary arteries; determining, by the computer system, vessel geometry of the identified one or more regions of the one or more coronary arteries, wherein the vessel geometry is determined based at least in part on a lumen wall and a vessel wall of the identified one or more regions of the one or more coronary arteries; determining, by the computer system, a pressure-flow rate relationship for the identified one or more regions of the one or more coronary arteries based on the determined vessel geometry and predetermined pressure-flow rate relationships for a plurality of vessel geometries, wherein the predetermined pressure-flow rate relationships are determined by: for each sample medical image of a plurality of sample medical images associated with a plurality of sample subjects: accessing the sample medical image; analyzing the sample medical image to identify a plurality of vessels depicted in the sample medical image; determining a geometry of each vessel of the plurality of vessels; generating a three-dimensional (3D)-printable model of the plurality of vessels, wherein the 3D-printable model comprises a file stored in a non-transitory computer-readable medium; accessing pressure measurements collected from a physical 3D model of the plurality of vessels produced by a 3D printer using the 3D-printable model, wherein the pressure measurements are collected by positioning a pressure sensor in a lumen of the physical 3D model at a plurality of locations within the physical 3D model, wherein the pressure measurements are collected at a plurality of inlet pressures, and wherein the pressure measurements are collected at a plurality of fluid flow rates; generating, by the computer system, an input for a machine learning model for determining a fluid flow characteristic, the input based at least in part on the determined vessel geometry and the pressure-flow rate relationship; determining, by the computer system using the machine learning model, a fluid flow characteristic of the identified one or more regions of the one or more coronary arteries, wherein the machine learning model is trained to determine the fluid flow characteristic using fluid flow measurements collected from a plurality of 3D printed models of coronary arteries of a plurality of sample subjects, wherein the machine learning model is trained to determine the fluid flow characteristic by: for each sample medical image of a plurality of sample medical images associated with a plurality of sample subjects: accessing the sample medical image; analyzing the sample medical image to identify a plurality of vessels depicted in the sample medical image; determining a geometry of each vessel of the plurality of vessels; generating a 3D-printable model of the plurality of vessels, wherein the 3D-printable model comprises a file stored in a non-transitory computer-readable medium; and accessing pressure measurements collected from a physical 3D model of the plurality of vessels produced by a 3D printer using the 3D-printable model, wherein the pressure measurements are collected by positioning a pressure sensor in a lumen of the physical 3D model at a plurality of locations within the physical 3D model, wherein the pressure measurements are collected at a plurality of inlet pressures, and wherein the pressure measurements are collected at a plurality of fluid flow rates; and providing the determined geometries and the pressure measurements to the machine learning model, wherein the determined geometries are used as inputs, and wherein the determined pressure measurements are used as outputs, wherein the machine learning model is trained to output a pressure pullback gradient curve; determining, by the computer system using the determined fluid flow characteristic of the identified one or more regions of the one or more coronary arteries, one or more fractional flow reserve values associated with one or more locations within the one or more regions of the one or more coronary arteries of the subject, wherein the computer system comprises a computer processor and an electronic storage medium.
Embodiment 2. The method of embodiment 1, wherein the one or more fractional flow reserve values are determined based at least in part on an intersection of a curve of distal pressure and a curve of coronary perfusion pressure as a function of coronary flow reserve.
Embodiment 3. A computer-implemented method for estimating fractional flow reserve for a subject based on image analysis of a medical image of the subject, the computer-implemented method comprising: accessing, by a computer system, the medical image of the subject, wherein the medical image of the subject depicts one or more regions of one or more coronary arteries of the subject; analyzing, by the computer system, the medical image of the subject to identify the one or more regions of the one or more coronary arteries; determining, by the computer system, vessel geometry of the identified one or more regions of the one or more coronary arteries, wherein the vessel geometry is determined based at least in part on a lumen wall and a vessel wall of the identified one or more regions of the one or more coronary arteries; determining, by the computer system, a pressure-flow rate relationship for the identified one or more regions of the one or more coronary arteries based on the determined vessel geometry and predetermined pressure-flow rate relationships for a plurality of vessel geometries; generating, by the computer system, an input for a machine learning model for determining a fluid flow characteristic, the input based at least in part on the determined vessel geometry and the pressure-flow rate relationship; determining, by the computer system using the machine learning model, a fluid flow characteristic of the identified one or more regions of the one or more coronary arteries, wherein the machine learning model is trained to determine the fluid flow characteristic using fluid flow measurements collected from a plurality of three-dimensional (3D) printed models of coronary arteries of a plurality of sample subjects; determining, by the computer system using the determined fluid flow characteristic of the identified one or more regions of the one or more coronary arteries, one or more fractional flow reserve values associated with one or more locations within the one or more regions of the one or more coronary arteries of the subject.
Embodiment 4. The computer-implemented method of embodiment 3, wherein the machine learning model is trained to determine the fluid flow characteristic by: for each sample medical image of a plurality of sample medical images associated with a plurality of sample subjects: accessing the sample medical image; analyzing the sample medical image to identify a plurality of vessels depicted in the sample medical image; determining a geometry of each vessel of the plurality of vessels; generating a 3D-printable model of the plurality of vessels, wherein the 3D-printable model comprises a file stored in a non-transitory computer-readable medium; and accessing pressure measurements collected from a physical 3D model of the plurality of vessels produced by a 3D printer using the 3D-printable model, wherein the pressure measurements are collected by positioning a pressure sensor in a lumen of the physical 3D model at a plurality of locations within the physical 3D model, wherein the pressure measurements are collected at a plurality of inlet pressures, and wherein the pressure measurements are collected at a plurality of fluid flow rates; and providing the determined geometries and the pressure measurements to the machine learning model, wherein the determined geometries are used as inputs and the machine learning model is trained to output the pressure measurements.
Embodiment 5. The computer-implemented method of embodiment 3 or 4, wherein the machine learning model is trained to output a pressure pullback gradient curve.
Embodiment 6. The computer-implemented method of any of embodiments 3-5, wherein the input includes information about a region of plaque detected in the one or regions of the one or more coronary arteries of the subject, wherein the information about the region of plaque includes one or more of: plaque length, plaque area, plaque volume, or plaque density.
Embodiment 7. The computer-implemented method of embodiment 6, wherein the plaque density is one or more of: low density non-calcified plaque, non-calcified plaque, or calcified plaque.
Embodiment 8. The computer-implemented method of embodiment 7, wherein the medical image is a coronary computed tomography angiography image, wherein low density non-calcified plaque corresponds to a radiodensity of between about −189 Hounsfield units (HU) and about 30 HU, wherein non-calcified plaque corresponds to a radiodensity of between about 31 HU and about 350 HU, and wherein calcified plaque corresponds to a radiodensity of between about 351 HU and about 2500 HU.
Embodiment 9. The computer-implemented method of any of claims embodiment 3-8, wherein the medical image is a coronary computed tomography angiography image.
Embodiment 10. The computer-implemented method of any of embodiments 3-9, wherein the wherein the one or more coronary arteries comprise one or more of left main (LM), ramus intermedius (RI), left anterior descending (LA D), diagonal one (D1), diagonal two (D2), left circumflex (Cx), obtuse marginal one (OM1), obtuse marginal two (OM2), left posterior descending (L-PDA), left posterolateral branch (L-PLB), right coronary (RCA), right posterior descending (R-PDA), or right posterolateral branch (R-PLB).
Embodiment 11. The computer-implemented method of embodiment 4, wherein at least one physical 3D model is printed using a plurality of materials, wherein at least one material of the plurality of materials is selected based on a density of a region of plaque identified in the sample medical image.
Embodiment 12. The computer-implemented method of embodiment 11, wherein the density of the region of plaque is determined based on a radiodensity of the region of plaque in the sample medical image, wherein the density is at least one of: low density non-calcified plaque, non-calcified plaque, or calcified plaque.
Embodiment 13. The computer-implemented method of any of embodiments 3-12, wherein a fractional flow reserve value of the one or more fractional flow reserve values is determined as a ratio of pressure distal to a region of plaque and pressure proximal to the region of plaque.
Embodiment 14. The computer-implemented method of any of embodiments 3-13, wherein the one or more fractional flow reserve values is determined for at least one of: a vessel, a vessel segment, or a vessel unit length.
Embodiment 15. A system for estimating fractional flow reserve for a subject based on image analysis of a medical image of the subject, the system comprising: at least one processor; and a computer-readable medium storing instructions that, when executed by the system, cause the system to: access the medical image of the subject, wherein the medical image of the subject depicts one or more regions of one or more coronary arteries of the subject; analyze the medical image of the subject to identify the one or more regions of the one or more coronary arteries; determine vessel geometry of the identified one or more regions of the one or more coronary arteries, wherein the vessel geometry is determined based at least in part on a lumen wall and a vessel wall of the identified one or more regions of the one or more coronary arteries; determine a pressure-flow rate relationship for the identified one or more regions of the one or more coronary arteries based on the determined vessel geometry and predetermined pressure-flow rate relationships for a plurality of vessel geometries; generate an input for a machine learning model for determining a fluid flow characteristic, the input based at least in part on the determined vessel geometry and the pressure-flow rate relationship; determine, using the machine learning model, a fluid flow characteristic of the identified one or more regions of the one or more coronary arteries, wherein the machine learning model is trained to determine the fluid flow characteristic using fluid flow measurements collected from a plurality of three-dimensional (3D) printed models of coronary arteries of a plurality of sample subjects; determine, using the determined fluid flow characteristic of the identified one or more regions of the one or more coronary arteries, one or more fractional flow reserve values associated with one or more locations within the one or more regions of the one or more coronary arteries of the subject.
Embodiment 16. The system of embodiment 15, wherein the machine learning model is trained to determine the fluid flow characteristic by: for each sample medical image of a plurality of sample medical images associated with a plurality of sample subjects: accessing the sample medical image; analyzing the sample medical image to identify a plurality of vessels depicted in the sample medical image; determining a geometry of each vessel of the plurality of vessels; generating a 3D-printable model of the plurality of vessels, wherein the 3D-printable model comprises a file stored in a non-transitory computer-readable medium; and accessing pressure measurements collected from a physical 3D model of the plurality of vessels produced by a 3D printer using the 3D-printable model, wherein the pressure measurements are collected by positioning a pressure sensor in a lumen of the physical 3D model at a plurality of locations within the physical 3D model, wherein the pressure measurements are collected at a plurality of inlet pressures, and wherein the pressure measurements are collected at a plurality of fluid flow rates; providing the determined geometries and the pressure measurements to the machine learning model, wherein the determined geometries are used as inputs and the machine learning model is trained to output the pressure measurements.
Embodiment 17. The system of embodiment 15 or 16, wherein the machine learning model is trained to output a pressure pullback gradient curve.
Embodiment 18. The system of embodiment 15, 16, or 17, wherein the input includes information about a region of plaque detected in the one or regions of the one or more coronary arteries of the subject, wherein the information about the region of plaque includes one or more of: plaque length, plaque area, plaque volume, or plaque density.
Embodiment 19. The system of embodiment 18, wherein the plaque density is one or more of: low density non-calcified plaque, non-calcified plaque, or calcified plaque.
Embodiment 20. The system of embodiment 19, wherein the medical image is a coronary computed tomography angiography image, wherein low density non-calcified plaque corresponds to a radiodensity of between about −189 Hounsfield units (HU) and about 30 HU, wherein non-calcified plaque corresponds to a radiodensity of between about 31 HU and about 350 HU, and wherein calcified plaque corresponds to a radiodensity of between about 351 HU and about 2500 HU.
Embodiment 21. The system of any of embodiments 15-20, wherein the medical image is a coronary computed tomography angiography image.
Embodiment 22. The system of any of embodiments 15-21, wherein the wherein the one or more coronary arteries comprise one or more of left main (LM), ramus intermedius (RI), left anterior descending (LAD), diagonal one (D1), diagonal two (D2), left circumflex (Cx), obtuse marginal one (OM1), obtuse marginal two (OM2), left posterior descending (L-PDA), left posterolateral branch (L-PLB), right coronary (RCA), right posterior descending (R-PDA), or right posterolateral branch (R-PLB).
Embodiment 23. The system of embodiment 16, wherein at least one physical 3D model is printed using a plurality of materials, wherein at least one material of the plurality of materials is selected based on a density of a region of plaque identified in the sample medical image.
Embodiment 24. The system of embodiment 23, wherein the density of the region of plaque is determined based on a radiodensity of the region of plaque in the sample medical image, wherein the density is at least one of: low density non-calcified plaque, non-calcified plaque, or calcified plaque.
Embodiment 25. The system of any of embodiments 15-24, wherein a fractional flow reserve value of the one or more fractional flow reserve values is determined as a ratio of pressure distal to a region of plaque and pressure proximal to the region of plaque.
Embodiment 26. The system of any of embodiments 15-25, wherein the one or more fractional flow reserve values is determined for at least one of: a vessel, a vessel segment, or a vessel unit length.

### Deep Learning Fractional Flow Reserve Estimation

As described herein, fractional flow reserve (FFR) is a diagnostic technique used to measure pressure differences in coronary arteries, for example across a coronary artery stenosis and can be used, for example, to estimate the likelihood that a stenosis significantly impedes oxygen delivery to cardiac muscle.

In conventional invasive FFR procedures, a catheter is inserted into a patient (e.g., via the femoral or radial arteries). A pressure sensor is used to measure pressure as the catheter is pulled back. Lower FFR values (e.g., higher pressure drops) can indicate that a lesion is more hemodynamically significant. Typically, a threshold FFR value of from about 0.75 to about 0.8 is used to differentiate between ischemic and non-ischemic lesions (e.g., an FFR value above the threshold FFR value is considered non-ischemic, while an FFR value below the threshold is considered ischemic). However, there is no absolute cutoff where an FFR value is considered abnormal. Moreover, low FFR values do not necessarily indicate ischemia. For example, even a healthy vessel can show significant pressure changes (and thus potentially lower FFR values) due to the tapering of vessels near the distal end. Additionally, different lesions can present differently and pose challenges in determining how, where, and even if intervention should be performed. For example, lesions can be relatively long or short, and in some cases, there may be multiple lesions. This can present challenges in deciding where to measure pressure values. At the distal end, a measured FFR can represent a combination of the effects of various lesions, which can be informative but may provide limited information about exactly where to perform an intervention as it lacks information about individual lesions. For example, when there are multiple stenoses, it may be unclear which of the stenoses are clinically significant.

As described herein, Fractional Flow Reserve-Computed Tomography (FFR-CT) is an alternative to invasive FFR, in which coronary flow is modeled from coronary CT angiography (CCTA) images. FFR-CT can involve complex computational fluid dynamics calculations. There can be many inputs when performing CFD calculations, such as inflow, aortic outlet, and coronary outlet boundary conditions. Various parameters can be used and/or optimized, such as the terminal resistance at the end of a vessel supplying the left ventricle, terminal resistance at the end of a vessel supplying the right ventricle, pressure difference between left and right ventricle beds due to left ventricle myocardium pressure, flow total estimate, left main artery cross-sectional area, allometric effects of volumes, lengths, and/or cross-sectional areas on total flow, resistance in stenoses due to turbulence, blood density, viscosity, and so forth. It can be difficult to determine boundary conditions, optimize parameters for specific patients, and so forth. Thus, obtaining accurate, clinically useful results using complex CFD calculations can be difficult.

Reduced order CFD can be used in some implementations and may enable more computationally efficient FFR calculations. Simplified, reduced order models (e.g., OD or 1D models) can be used to carry out CFD calculations. Various assumptions can be made depending upon the specific implementation. For example, vessels can be treated as rigid structures in some implementations. While reduced order models can eliminate considerable complexity and many parameters, such simplification can come at the cost of clinical accuracy, as simplifications, assumptions such as boundary conditions, and so forth can significantly impact CFD results.

In some cases, vessels can be straightened, and calculations can be carried out on the straightened vessels. However, such approaches can offer limited insight as the tortuosity of vessels can play an important role in the dynamics of fluids moving inside the vessels. For example, a fluid may behave very differently in a vessel with a turn or kink than it would inside a vessel with the same diameter but which lacks any turns.

In conventional approaches to using computational fluid dynamics to simulate fractional flow reserve based on CCTA images, there can be significant errors, for example due to the determination of boundary conditions and parameter optimization for patient-specific simulation, which often requires intensive computational power and time. Previous methods using deep learning approaches have had several disadvantages. For example, some deep learning approaches provide only a single FFR value per patient or per artery. Some approaches have proposed using deep learning-based methods to predict FFR pullback measurements along an artery. However, such approaches have used 1D anatomical measurement data extracted from the vessel as inputs to the neural networks to generate predictions. This can provide inaccurate predictions because such an approach does not utilize holistic features including, for example, vascular morphology, stenosis, and atherosclerosis, all of which can contribute significantly to hemodynamically significant coronary artery disease.

Accordingly, there is a need for improved methods that can combine the simplicity of approaches such as reduced order CFD while still delivering accurate, clinically actionable results.

In some implementations, as described herein, deep learning can be used to identify ischemia, to produce FFR pullback curves, and so forth. In some implementations, multiple inputs can be provided to a deep learning model and used to generate clinically relevant outputs. For example, in some implementations, 3D curved multiplanar reconstruction (CM PR), reduced order CFD, and 3D tortuosity estimation can be provided to a deep learning model. In some implementations, additionally or alternatively, information such as a 3D plaque map, 3D wall shear stress map, myocardial mass at risk (M MAR) measurements, etc., can be provided to the deep learning model. In some implementations, different reconstructions (e.g., SM PR), different forms of computational fluid dynamics data, and/or different tortuosity information (e.g., 3D tortuosity estimates) can be provided to a model. In some implementations, the inputs can be provided to a deep learning model that is configured to output one or more of: a continuous FFR pullback curve; discrete CFD outputs at one or more of the ostium, distal vessel, a location before a lesion, or a location after a lesion; and/or a binarized ischemia prediction (e.g., ischemic or not ischemic, or likely ischemic or likely not ischemic). In some implementations, the deep learning model can be configured to generate ischemia predictions in more than two categories, which can include, for example, an indeterminate prediction, which can be appropriate if the model's decisions do not strongly point to ischemia or not. In some implementations, a continuous FFR pullback curve can be quasi-continuous. For example, the FFR pullback curve can have a relatively small step size. In some embodiments, the step size for the FFR pullback curve can be limited by the pixel size in a CCTA image or other representation.

By providing multiple types of input data and producing multiple outputs with a single model, more reliable and/or accurate outputs can be obtained, while still enjoying the benefits of simplified calculations such as reduced order CFD. Moreover, since multiple outputs can be produced by a single model, the outputs can be consistent with one another. In contrast, in some conventional approaches, when calculations are performed separately on different sources of data, the different calculations can provide results that are in conflict with one another. For example, a first calculation may indicate ischemia, while a second, different calculation may not indicate ischemia. Approaches that consider only a single or otherwise limited number of data types may be more prone to inaccuracy because, for example, less information is considered when generating an output, some correlations and interactions between different features may not be considered, and so forth.

In some implementations, a reduced order CFD calculation can include a OD or 1D CFD calculation. In some implementations, the CFD calculation can be a relatively simple flow gradient simulation that assumes rigid walls. In some implementations, such a reduced order CFD calculation can, when combined with other data, be used to make determinations that are more accurate than determinations that may be made by a model that considers reduced order CFD calculations alone.

Various techniques can be used to visualize coronary arteries in CCTA images, such as maximum intensity projection, volume render techniques, stretched/straightened multiplanar reconstruction (SM PR), curved multiplanar reconstruction (CM PR), and so forth. In some implementations, different visualization techniques may be used in making FFR predictions. In some implementations, some techniques may result in better predictions than some other techniques. For example, CM PR may typically result in better predictions than SM PR, for example because data about the curvature of the vessels is maintained. Some techniques may be associated with higher computational demands than some other techniques. For example, CM PR may, in some cases, require more computational resources than SM PR.

The use of simplified models or reconstructions in isolation can have several drawbacks. For example, the use of SM PR imaging can fail to account for the significant effects that curvature of the vessels can have on flow. CFD, as described herein, can be prohibitively computationally intensive, and it can be difficult to determine appropriate boundary conditions. Reduced order CFD can simplify calculations, but doing so comes at a cost of result accuracy. For example, a reduced order CFD calculation may not consider that the vessels are not rigid but rather can expand, contract, and/or shift in response to pumping action and fluid flow. However, reliable results can be achieved when multiple sources of information are used together as inputs into a deep learning model, even if the underlying information sources use simplified models, reconstructions, etc.

In some implementations, a deep learning model can be trained to provide multiple outputs, which can, for example, enable an interventionist to use the model outputs to perform varying levels of analysis or investigation. For example, in some implementations, a system can be configured to provide outputs that allow an interventionist to review high level predictions (e.g., classification as ischemic or not ischemic) and then dive into the outputs for further investigation. For example, in some implementations, a deep learning model can be trained to output a classification (e.g., likely ischemic or likely not ischemic), an FFR pullback curve, and/or discrete FFR values at locations along a vessel, such as just before or just after a lesion.

While determining ischemia can be at least in part an objective process, expert analysis can play an important role, as experts may have developed insights through years of experience that enable them to make better decisions. In some implementations, a model can be trained using training data that is labeled with expert conclusions. The expert conclusions can be, for example, conclusions made by physicians after reviewing imaging data, conventional FFR data, FFR-CT data, and so forth. Training using expert conclusions can be important because, as described herein, there may not be clear numerical cutoffs where lesions are deemed ischemic or not ischemic. Rather, experts can form opinions based on a wide range of information and experience.

In some approaches to machine learning, large data sets are used without careful selection of input parameters. The machine learning model then goes through training to determine weights that give different parameters different importance in determining the outputs. Such an approach can work well when there is a large amount of training data available. However, such an approach can deliver poor results (e.g., the trained model may output results with significant errors) when there is limited training data available. For example, training a machine learning model with limited data can lead to overfitting, where the model becomes optimized for the training data but does not learn general patterns. For example, if only limited training data is available, the model can become overfitted to the training data, where the model learns to fit the noise in the data rather than the underlying patterns. Depending upon the model, the available data, and so forth, various techniques can be used to mitigate such issues. For example, in some embodiments, techniques such as data augmentation, regularization, and/or transfer learning can be used. In some embodiments, simpler models may be more generalizable with limited training data than more complex models.

In some embodiments, features used for training can be carefully curated based on their relevance, predictive power, and/or the like. In some embodiments, a model can be initially trained using FFR-CT results, which may be more numerous than invasive FFR results. For example, during initial training, a relatively large number of FFR-CT results can be used for training (e.g., about 1000 vessels, with about 800 for training and about 200 for testing). In some embodiments, the model can be fine-tuned using a smaller number of invasive FFR results (e.g., about 100 vessels). In some embodiments, rather than or in addition to FFR-CT results, a model can be trained using FFR 3D results as may be determined using the approaches described herein, and/or can be trained using measurement data collected from 3D printed models of coronary arteries.

In some embodiments, features used as inputs to a model can be selected based at least in part on clinical prior knowledge, e.g., features that physicians have identified as important. It will be appreciated, however, that a machine learning model as described herein can offer a level of analysis that would be infeasible for a human physician to do. For example, a physician typically only considers one or a few features, and does not consider the complex, and possibly unknown, interplay between different features, between different types of data, and so forth. It is often not clear, for example, how an anatomical measure relates to a functional measure. As an example, it may be known that the presence of a lesion in a turn in a vessel can be more impactful than a lesion in a relatively straight portion of a vessel, but it may be difficult or impossible for a physician to consider all or even more than a handful of factors that can influence how impactful the location of a plaque is likely to be.

While in some embodiments, limited feature sets are considered and calculations are carried out on, for example, extracted vessels, it will be appreciated that in some embodiments, greater numbers of features and/or different features can be used. In some embodiments, a level of pre-processing can be different. For example, if there are enough available CCTA images, a model can be trained to operate on CCTA images rather than extracted vessels. That is, extracting vessels from CCTA images as a separate step or steps in a process may not be necessary.

FIG. 26 is a block diagram that illustrates an example of ischemia prediction according to some embodiments. In FIG. 26, a computer system can be configured to extract a coronary artery tree, for example based on CCTA data. The coronary arteries can have associated therewith various data including, for example, a 3D plaque map, reduced order model computational fluid dynamics data, a 3D curved multiplanar reconstruction image, 3D tortuosity data, a 3D wall shear stress map, and/or other data. The various types of data can be provided to a machine learning model (e.g., a deep learning model) that can predict, for example, whether or not the data shows presence of ischemia, FFR pullback curves, and/or discrete FFR values. A practitioner can analyze the outputs to determine if a patient exhibits signs of ischemia or not. In some embodiments, the clinician can perform further investigation, for example to analyze FFR pullback curves, to evaluate FFR values at specific locations within a vessel, and so forth. The clinician can use this information to, for example, determine whether or not pharmaceutical intervention, surgical intervention, both, or neither is indicated. If surgical intervention is indicated, the clinician can analyze the outputs when developing a treatment plan. For example, the clinician can review discrete FFR values before and after lesions, FFR pullback curves across lesions, and so forth to determine which lesions to treat, for example by stenting. While described in terms of CM PR data, 3D tortuosity data, etc., it will be appreciated that the process shown in FIG. 26 can be carried out using two-dimensional data.

FIG. 27 is a block diagram that illustrates an example process for determining various ischemia-related information using a machine learning model according to some implementations. The process 2700 can be performed on a computer system. At operation 2710, the system can access image data (e.g., 3D CMPR data). At operation 2720, the system can access computational fluid dynamics data (e.g., reduced order computational fluid dynamics data). In some implementations, the system can perform a reduced order CFD calculation to obtain the reduced order CFD data. At operation 2730, the system can receive tortuosity data (e.g., 3D tortuosity data). At operation 2740, the system can provide the image data, CFD data, and tortuosity data to a machine learning model. For example, in some implementations, the system can generate a feature vector that encodes the CM PR data, the reduced order CFD data, and the 3D tortuosity data. In some implementations, the system can provide additional data, such as 3D plaque map data and/or 3D wall shear stress map data. At operation 2750, the system can, using the machine learning model, determine a likelihood of ischemia. If the likelihood is at or above a threshold value (e.g., due to similarity in the data to other vessels with clinically significant ischemia), the system can determine that ischemia is likely. If the likelihood is below the threshold value, the system can determine that ischemia is not likely. In some embodiments, the process can stop if ischemia is determined not to be likely; in other embodiments the process can continue as there may still be significance in determining FFR values. At operation 2760, the system can determine one or more discrete FFR values, for example at the proximal and/or distal ends of a lesion. At operation 2770, the system can determine an FFR pullback curve. In some implementations, the system can determine the FFR pullback curve prior to determining the discrete FFR values. In some embodiments, the discrete FFR values can be extracted from the FFR pullback curve. In some implementations, the FFR pullback curve can be a continuous FFR pullback curve. As used herein, the term “continuous” can mean that the FFR pullback curve is computed with a step size at or below a threshold value. While described in terms of CM PR data, 3D tortuosity data, etc., it will be appreciated that the process shown in FIG. 27 can be carried out using two-dimensional data. Automatic Vessel Labeling

Identifying and labeling coronary arteries in coronary computed tomography angiography (CCTA) images can be a significant challenge. For example, coronary arteries can be small and intricately arranged throughout the heart muscle. Extensive branching and anatomical variation can make identification and labeling difficult. Moreover, some patients may not conform to typical branching patterns.

Accurate identification and labeling of coronary arteries can be important or even required for certain downstream processes, for accurate diagnosis, for surgical planning, and so forth. Errors in identification and labeling can lead to misinterpretation of image, incorrect diagnosis, or incorrect treatment decisions, which can in some cases result in patient harm or otherwise sub-optimal patient outcomes.

In some embodiments, a machine learning model can be trained to automatically extract and label coronary arteries, for example as depicted in a CCTA image, to automatically extract and label the coronary arteries. In some embodiments, the machine learning algorithm can incorporate prior knowledge of vessel anatomy. For example, prior knowledge can include known typical relationships between vessels, known person-to-person variations in anatomy, and so forth.

In some embodiments, a computer system can be configured to run a machine learning model to label coronary arteries in CCTA images.

In some embodiments, a coronary artery tree can be extracted from a CCTA image. In some embodiments, the coronary artery tree can be represented as a mesh. In some embodiments, the coronary artery tree can be represented as a point. In some embodiments, a point transformer (or other suitable algorithm) can be trained to model vessels in the coronary artery tree. In some embodiments, anatomical prior knowledge can be incorporated via post-processing. In some embodiments, a Viterbi algorithm can be used in post-processing. For example, along each vessel, a transition probability matrix can be created or referenced based on the vessel anatomy to ensure valid labeling transitions. For example, left main artery to left main descending artery is logical, while a transition from the first diagonal branch (D1) of the left anterior descending (LA D) artery to the second diagonal branch (D2) is anatomically illogical. Such post-processing can identify potential mistakes made by the point transformer model (or other suitable model) in labeling the arteries. In some cases, such errors may indicate a failure of the model. In other cases, errors may be indicative of anatomical abnormalities that may warrant further investigation by a physician.

In some embodiments, a point transformer model (or other suitable model) can receive a coronary artery tree. In some embodiments, the model can label the vessels. In some embodiments, the coronary artery tree can be sub-sampled. For example, the coronary artery tree can be converted into units of millimeters, and subsamples can be taken every 1 mm. This is merely an example, and other suitable subsample size can be used. In some embodiments, the subsample size can be limited by a resolution or slice thickness of an image used for generating the coronary artery tree. In some embodiments, the coronary artery tree can be represented as slices having a fixed thickness, for example 0.25 mm), and the sub-sampling step size can be defined in terms of number of slices (e.g., four slices for a slice size of 0.25 mm and a step size of 1 mm). While in some embodiments, vessel labeling can be carried out by analyzing slices, in other embodiments, vessel labeling is based on, for example, analysis of a 3D volume.

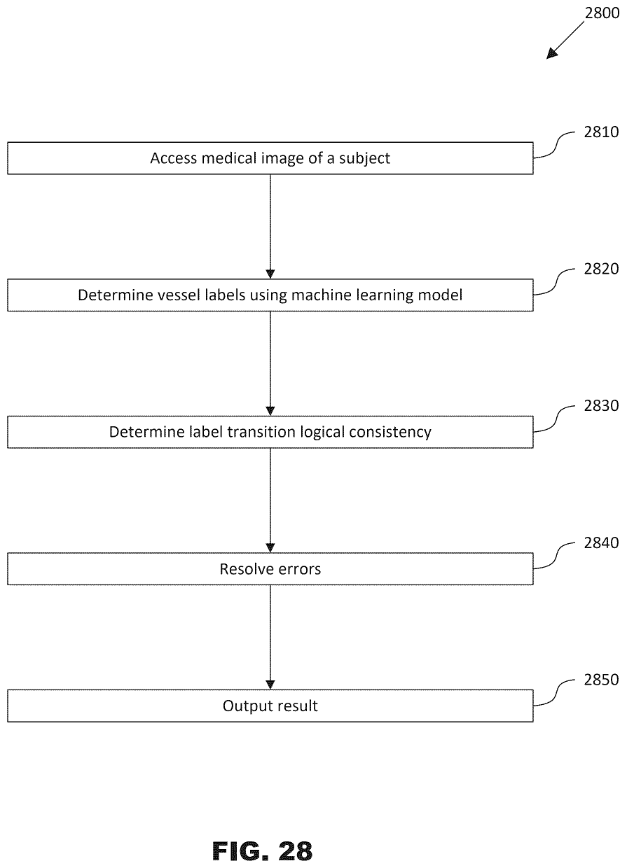

FIG. 28 is a flowchart of an example method for determining and checking the correctness of vessel labeling according to some embodiments. The process 2800 illustrated in FIG. 28 can be carried out on a computer system.

At operation 2810, the system can access a medical image of a subject, for example a CCTA image of a subject. In some embodiments, the medical image can comprise a coronary artery tree extracted from an image, such as a CCTA image.

At operation 2820, the system can determine vessel labels using a machine learning model. In some embodiments, the machine learning model can determine a probability associated with a label. In some embodiments, the machine learning model can determine a plurality of labels, each label having a probability associated therewith. For example a vessel can be assigned more than one label, and each of the labels can have an associated probability which may indicate a level of confidence that the particular label is correct for the vessel.

At operation 2830, the system can determine label transition logical consistency, for example using an algorithm such as a Viterbi algorithm and known anatomical relationships.

At operation 2840, the system can, if there are any errors or inconsistencies, resolve the errors, for example by prompting the user to correct an error by selecting a correct label. In some embodiments, selection options can be ordered based on label probabilities determined by the machine learning model. In some embodiments, selection options can be limited based on anatomical consistency. In some embodiments, the system can automatically determine a label to resolve the inconsistency.

At operation 2850, the system can output results (e.g., labels). In some embodiments, the system can output a labeled image, a JSON file, and/or an XML file, or any other suitable output.

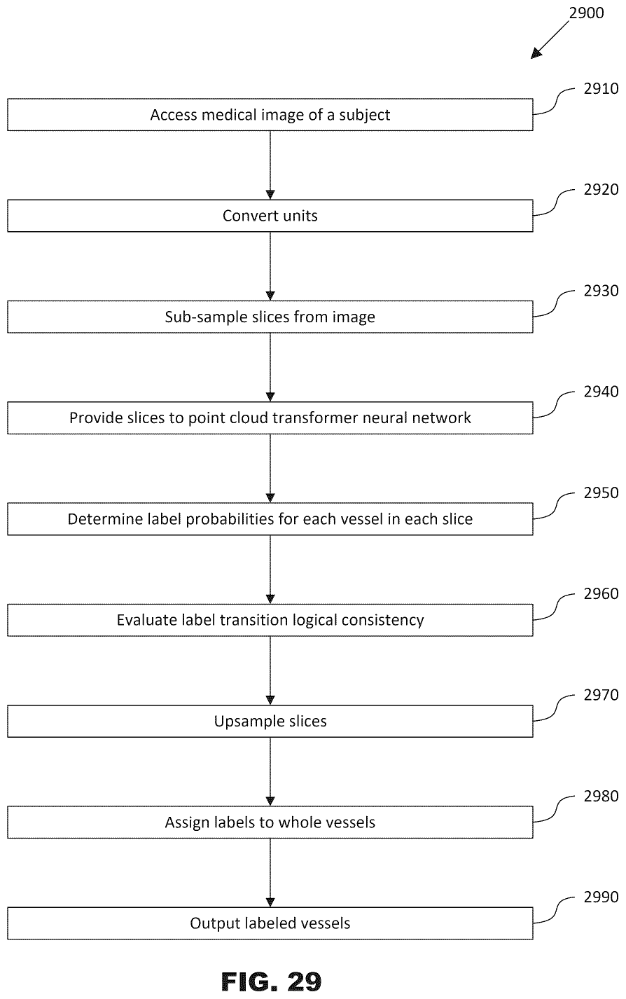

FIG. 29 is a flowchart of an example method for determining and checking the correctness of vessel labeling according to some embodiments. The process 2900 illustrated in FIG. 29 can be carried out on a computer system.

At operation 2910, the system can access a medical image of a subject, for example a CCTA image. At operation 2920, the system can convert one or more units of the image. For example, the system can determine units in millimeters or another unit of measurement, which can be beneficial as it describes a physical distance rather than, for example, measurements in pixels, which may represent any physical distance depending upon the scale. At operation 2930, the system can sub-sample slices from the image. For example, the system can sample a slice every 0.5 mm, 1 mm, 2 mm, 3 mm, 4 mm, etc. At operation 2940, the system can provide the slices to a point cloud transformer neural network or other suitable machine learning model. At operation 2950, the point cloud transformer or other model can determine label probabilities for each vessel in each slice.

At operation 2960, the system can evaluate label transition logical consistency, for example using a Viterbi algorithm.

At operation 2970, the system can upsample the slices. For example, if a slice thickness is 0.25 mm, and samples were taken every fourth slice, the samples can be upsampled to four slices.

At operation 2980, the system can assign labels to whole vessels, for example based on the labels determined at operation 2950 and checked for consistency at operation 2960. At operation 2990, the system can provide an output, such as a labeled image, JSON file, XML file, or any other suitable output.

### Calcium Blooming

As described herein, coronary computed tomography angiography (CCTA) is a non-invasive imaging technique that can be used to identify plaque in coronary vessels, and different types of plaque (e.g., calcified vs. non-calcified) can have significantly different clinical significance. Additionally, the size and shape of plaque are significant drivers of clinical significance. Thus, there is a need to accurately identify and quantify different types of plaque.

M any approaches to classifying plaque as calcified or non-calcified have relied on fixed cutoff values. For example, 350 Hounsfield Units (HU) is typically used as the lower clinical threshold for identifying calcified plaque, with any plaque having a value of equal to or greater than 350 HU being classified as calcified plaque. However, this likely overestimates the true calcified plaque volume in some cases, and may underestimate calcified plaque in other cases, for example depending on specific scan parameters, patient anatomy, and so forth.

A significant challenge with analyzing CT images is calcium blooming. Calcium blooming is a phenomenon that occurs in CT imaging studies, where the presence of dense calcified structures, such as calcified plaque in the coronary arteries, causes imaging artifacts that exaggerate the size and density of such structures. Calcium blooming has been attributed to various causes, including partial volume averaging, motion, and beam hardening. Partial volume averaging can occur because of the limited spatial resolution of a CT scanner and can be influenced by factors such as detector cell size, focal spot size, azimuthal blur, crosstalk, and the reconstruction algorithm used, which can introduce blooming due to interpolation or other types of processing.

Various techniques and hardware solutions have been developed to address calcium blooming, such as high-resolution imaging hardware, high resolution reconstruction that can preserve edges, dual energy CT or dual contrast CT, and various post-processing algorithms. However, these approaches can each add significant complexity, cost, or both. Moreover, these approaches do not address the issue of analyzing images that exhibit calcium blooming. That is, these techniques and hardware solutions may prevent or reduce calcium blooming, but do not address the problem of analyzing an image that does exhibit blooming.

As described herein, using a variable calcified plaque threshold can help to mitigate calcium blooming issues. Calcium blooming can be affected by scan parameters such as peak kilovoltage (kVp), current (mA), and so forth, imaging technology (e.g., photon counting CT vs. multi-detector CT), reconstruction algorithm, patient factors (e.g., sex, body mass index, calcified plaque burden, total plaque burden), or any combination thereof.

In some embodiments, a plurality of CT images (e.g., for multiple patients) is analyzed (e.g., using a machine learning model) and various parameters such as volume, length, area, geometry, etc., for total plaque, calcified plaque, non-calcified plaque, low attenuation plaque (which can be a sub-category of non-calcified plaque), etc., can be determined. Any combination of these and/or other parameters can be determined. In some embodiments, the parameters determined from CT analysis are compared with reference parameters determined from invasive techniques such as intravascular ultrasound (IV US), optical coherence tomography (OCT), or both. Other techniques can be used additionally or alternatively. It will be appreciated that the reference parameters are not necessarily correct (that is, a plaque volume determined from OCT may not equal the true plaque volume); however, such reference parameters can be useful as they are widely used in the medical field.

This analysis and comparison can be used to determine how to adjust a calcified plaque threshold (e.g., in Hounsfield Units (HU)) to mitigate the effects of calcium blooming and determine calcium parameters that are more closely aligned with those determined by invasive techniques such as IVUS or OCT.

One approach is to generate a table (e.g., a multi-dimensional table) that indicates an optimized calcified plaque threshold based on one or more input factors. The input factors can include scan parameters, reconstruction algorithm, patient factors, or any combination thereof. Such an approach can provide certain benefits. For example, applying the table to a new image is computationally easy, as determining the calcified plaque threshold can be accomplished by looking up certain parameters in the table and identifying the calcified plaque threshold associated with that particular combination of parameters. Such an approach can also offer a relatively high degree of explainability, as the parameters that affect the calcified plaque threshold are concretely defined, and their impacts are both known and fixed. In some cases, an input factor value may not match a value included in the table. In some embodiments, a system can be configured to use interpolation methods when determining a plaque calcification threshold for input parameter values that are not included in the table.

In some circumstances, a relatively simple table can be useful. However, a lookup table can have significant drawbacks. For example, when interpolating, errors can be made if the dependence of the calcified plaque threshold on a particular input factor is incorrect, for example if a linear relationship is assumed when the relation is in fact quadratic or exponential. When there are many input factors, a table can become relatively sparse if the data used to generate the table is not sufficiently large or diverse, which can lead to a greater need to estimate or interpolate, potentially resulting in significant errors in the determined calcified plaque threshold.

To be manageable and/or relatively densely populated, a table may include only a subset of possible input factors. For example, in some embodiments, only the input factors with the greatest observed impact on calcified plaque threshold are included in the table. While such an approach may work well in many cases, in other cases, it may fail as certain factors are ignored. Additionally, input factors may not be independent of one another. There can be complex interplay between input factors that may not be fully represented in the table.

Accordingly, it can be significant to utilize other, potentially non-static techniques for determining calcified plaque thresholds. A machine learning model can be configured to determine calcified plaque thresholds. Machine learning models can have many advantages. For example, a machine learning model can be more scalable and flexible as compared with a fixed table that may have limited data, limited parameters, or both. A machine learning model can handle a large number of variables and the complex relationships between them and can easily scale to include new data and variables without significant rework. In contrast, a table becomes unwieldy and difficult to manage as the number of variables and possible combinations increases, and it can be difficult to add new variables. For example, there may be limited data available such that the table becomes unacceptably sparse when a new variable is added. A machine learning model can generalize from training data to make predictions on new, unseen data. That is, a machine learning model can provide reasonable outputs (e.g., reasonable calcified plaque thresholds) even for combinations of variables that were not present in the training data. In contrast, a lookup table is not generalized and limited approaches such as interpolation may be used when particular input variable values are not included in the table. Machine learning models, on the other hand, can capture and model complex, non-linear relationships and interactions between variables, in some cases even when there is limited training data available.

As described herein, a table can be relatively easy to create and use, at least so long as the table is fairly simple. In contrast, a machine learning model can undergo a potentially computationally intensive training process before being deployed. However, once deployed, a machine learning model may provide better performance than a table, particularly in cases where the table includes many input variables, which can require searching through a large dataset to determine an appropriate plaque calcification threshold for a given set of input variable values. Moreover, when the table is used in conjunction with rigid techniques such as linear interpolation, a table may produce inferior results, slower results, or both.

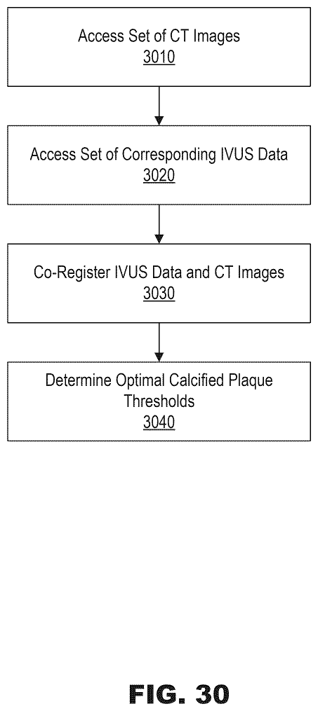

FIG. 30 is a flowchart that illustrates an example process for determining calcified plaque thresholds according to some embodiments. At operation 3010, a system can access a set of CT images. At operation 3020, the system can access a set of corresponding IVUS data. At operation 3030, the system can co-register the IVUS data and the CT images. At operation 3040, the system can determine optimal calcified plaque thresholds for each image in the set of CT images.

A calcified plaque threshold can be 400 HU or about 400 HU, 401 HU or about 401 HU, 402 HU or about 402 HU, 403 HU or about 403 HU, 404 HU or about 404 HU, 405 HU or about 405 HU, 406 HU or about 406 HU, 407 HU or about 407 HU, 408 HU or about 408 HU, 409 HU or about 409 HU, 410 HU or about 410 HU, 411 HU or about 411 HU, 412 HU or about 412 HU, 413 HU or about 413 HU, 414 HU or about 414 HU, 415 HU or about 415 HU, 416 HU or about 416 HU, 417 HU or about 417 HU, 418 HU or about 418 HU, 419 HU or about 419 HU, 420 HU or about 420 HU, 421 HU or about 421 HU, 422 HU or about 422 HU, 423 HU or about 423 HU, 424 HU or about 424 HU, 425 HU or about 425 HU, 426 HU or about 426 HU, 427 HU or about 427 HU, 428 HU or about 428 HU, 429 HU or about 429 HU, 430 HU or about 430 HU, 431 HU or about 431 HU, 432 HU or about 432 HU, 433 HU or about 433 HU, 434 HU or about 434 HU, 435 HU or about 435 HU, 436 HU or about 436 HU, 437 HU or about 437 HU, 438 HU or about 438 HU, 439 HU or about 439 HU, 440 HU or about 440 HU, 441 HU or about 441 HU, 442 HU or about 442 HU, 443 HU or about 443 HU, 444 HU or about 444 HU, 445 HU or about 445 HU, 446 HU or about 446 HU, 447 HU or about 447 HU, 448 HU or about 448 HU, 449 HU or about 449 HU, 450 HU or about 450 HU, 451 HU or about 451 HU, 452 HU or about 452 HU, 453 HU or about 453 HU, 454 HU or about 454 HU, 455 HU or about 455 HU, 456 HU or about 456 HU, 457 HU or about 457 HU, 458 HU or about 458 HU, 459 HU or about 459 HU, 460 HU or about 460 HU, 461 HU or about 461 HU, 462 HU or about 462 HU, 463 HU or about 463 HU, 464 HU or about 464 HU, 465 HU or about 465 HU, 466 HU or about 466 HU, 467 HU or about 467 HU, 468 HU or about 468 HU, 469 HU or about 469 HU, 470 HU or about 470 HU, 471 HU or about 471 HU, 472 HU or about 472 HU, 473 HU or about 473 HU, 474 HU or about 474 HU, 475 HU or about 475 HU, 476 HU or about 476 HU, 477 HU or about 477 HU, 478 HU or about 478 HU, 479 HU or about 479 HU, 480 HU or about 480 HU, 481 HU or about 481 HU, 482 HU or about 482 HU, 483 HU or about 483 HU, 484 HU or about 484 HU, 485 HU or about 485 HU, 486 HU or about 486 HU, 487 HU or about 487 HU, 488 HU or about 488 HU, 489 HU or about 489 HU, 490 HU or about 490 HU, 491 HU or about 491 HU, 492 HU or about 492 HU, 493 HU or about 493 HU, 494 HU or about 494 HU, 495 HU or about 495 HU, 496 HU or about 496 HU, 497 HU or about 497 HU, 498 HU or about 498 HU, 499 HU or about 499 HU, 500 HU or about 500 HU, 501 HU or about 501 HU, 502 HU or about 502 HU, 503 HU or about 503 HU, 504 HU or about 504 HU, 505 HU or about 505 HU, 506 HU or about 506 HU, 507 HU or about 507 HU, 508 HU or about 508 HU, 509 HU or about 509 HU, 510 HU or about 510 HU, 511 HU or about 511 HU, 512 HU or about 512 HU, 513 HU or about 513 HU, 514 HU or about 514 HU, 515 HU or about 515 HU, 516 HU or about 516 HU, 517 HU or about 517 HU, 518 HU or about 518 HU, 519 HU or about 519 HU, 520 HU or about 520 HU, 521 HU or about 521 HU, 522 HU or about 522 HU, 523 HU or about 523 HU, 524 HU or about 524 HU, 525 HU or about 525 HU, 526 HU or about 526 HU, 527 HU or about 527 HU, 528 HU or about 528 HU, 529 HU or about 529 HU, 530 HU or about 530 HU, 531 HU or about 531 HU, 532 HU or about 532 HU, 533 HU or about 533 HU, 534 HU or about 534 HU, 535 HU or about 535 HU, 536 HU or about 536 HU, 537 HU or about 537 HU, 538 HU or about 538 HU, 539 HU or about 539 HU, 540 HU or about 540 HU, 541 HU or about 541 HU, 542 HU or about 542 HU, 543 HU or about 543 HU, 544 HU or about 544 HU, 545 HU or about 545 HU, 546 HU or about 546 HU, 547 HU or about 547 HU, 548 HU or about 548 HU, 549 HU or about 549 HU, 550 HU or about 550 HU, 551 HU or about 551 HU, 552 HU or about 552 HU, 553 HU or about 553 HU, 554 HU or about 554 HU, 555 HU or about 555 HU, 556 HU or about 556 HU, 557 HU or about 557 HU, 558 HU or about 558 HU, 559 HU or about 559 HU, 560 HU or about 560 HU, 561 HU or about 561 HU, 562 HU or about 562 HU, 563 HU or about 563 HU, 564 HU or about 564 HU, 565 HU or about 565 HU, 566 HU or about 566 HU, 567 HU or about 567 HU, 568 HU or about 568 HU, 569 HU or about 569 HU, 570 HU or about 570 HU, 571 HU or about 571 HU, 572 HU or about 572 HU, 573 HU or about 573 HU, 574 HU or about 574 HU, 575 HU or about 575 HU, 576 HU or about 576 HU, 577 HU or about 577 HU, 578 HU or about 578 HU, 579 HU or about 579 HU, 580 HU or about 580 HU, 581 HU or about 581 HU, 582 HU or about 582 HU, 583 HU or about 583 HU, 584 HU or about 584 HU, 585 HU or about 585 HU, 586 HU or about 586 HU, 587 HU or about 587 HU, 588 HU or about 588 HU, 589 HU or about 589 HU, 590 HU or about 590 HU, 591 HU or about 591 HU, 592 HU or about 592 HU, 593 HU or about 593 HU, 594 HU or about 594 HU, 595 HU or about 595 HU, 596 HU or about 596 HU, 597 HU or about 597 HU, 598 HU or about 598 HU, 599 HU or about 599 HU, 600 HU or about 600 HU, 601 HU or about 601 HU, 602 HU or about 602 HU, 603 HU or about 603 HU, 604 HU or about 604 HU, 605 HU or about 605 HU, 606 HU or about 606 HU, 607 HU or about 607 HU, 608 HU or about 608 HU, 609 HU or about 609 HU, 610 HU or about 610 HU, 611 HU or about 611 HU, 612 HU or about 612 HU, 613 HU or about 613 HU, 614 HU or about 614 HU, 615 HU or about 615 HU, 616 HU or about 616 HU, 617 HU or about 617 HU, 618 HU or about 618 HU, 619 HU or about 619 HU, 620 HU or about 620 HU, 621 HU or about 621 HU, 622 HU or about 622 HU, 623 HU or about 623 HU, 624 HU or about 624 HU, 625 HU or about 625 HU, 626 HU or about 626 HU, 627 HU or about 627 HU, 628 HU or about 628 HU, 629 HU or about 629 HU, 630 HU or about 630 HU, 631 HU or about 631 HU, 632 HU or about 632 HU, 633 HU or about 633 HU, 634 HU or about 634 HU, 635 HU or about 635 HU, 636 HU or about 636 HU, 637 HU or about 637 HU, 638 HU or about 638 HU, 639 HU or about 639 HU, 640 HU or about 640 HU, 641 HU or about 641 HU, 642 HU or about 642 HU, 643 HU or about 643 HU, 644 HU or about 644 HU, 645 HU or about 645 HU, 646 HU or about 646 HU, 647 HU or about 647 HU, 648 HU or about 648 HU, 649 HU or about 649 HU, or 650 HU or about 650 HU. In some embodiments, the calcified plaque threshold can be any value between these values, or more or less. In some embodiments, a calcified plaque threshold can encompass any range of values between and/or including the listed values.

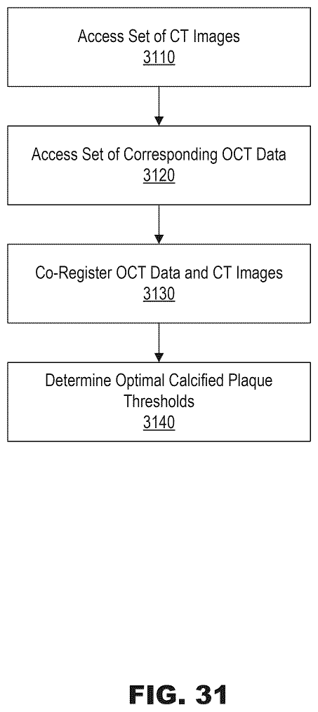

FIG. 31 is a flowchart that illustrates another example process for determining calcified plaque thresholds according to some embodiments. At operation 3110, a system can access a set of CT images. At operation 3120, the system can access a set of corresponding OCT data. At operation 3130, the system can co-register the OCT data and the CT images. At operation 3140, the system can determine optimal calcified plaque thresholds for each image of the set of CT images.

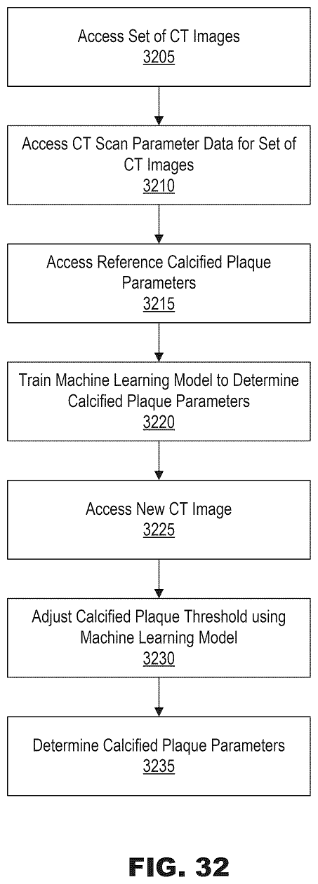

FIG. 32 is a flowchart that illustrates an example process for training and deploying a machine learning model for calcified plaque characterization according to some embodiments. At operation 3205, a system can access a set of CT images. At operation 3210, the system can access CT scan parameter data for the set of CT images. The CT scan parameter data can include, for example, kVp, mA, etc. In some embodiments, the system can access patient data, such as sex, gender, body mass index, and so forth. At operation 3215, the system can access a set of reference calcified plaque parameters. The calcified plaque parameters can include, for example, volume, area, angle, etc. The reference plaque parameters can be parameters determined using a technique such as IVUS or OCT. In some cases, the reference plaque parameters can comprise parameters collected using multiple techniques, such as a combination of IVUS and OCT.

At operation 3220, the system can train a machine learning model to determine calcified plaque parameters. For example, the system can train the machine learning model using supervised learning. For example, images can be labeled with plaque parameters derived from IVUS and/or OCT, and the machine learning model can be trained to determine calcified plaque thresholds that cause plaque parameters determined from a CCTA image to more closely matched those produced using IVUS and/or OCT.

After the model is trained, the model can be deployed to analyze new CCTA images. At operation 3225, the system can access a new CT image. The new CT image can be an image of a new patient or a new image (e.g., an image not included in training data) of an existing patient. At operation 3230, the system can adjust a calcified plaque threshold associated with the image based on the determined calcified plaque parameters. At operation 3235, the system can, using a second machine learning model, determine calcified plaque parameters for the patient.

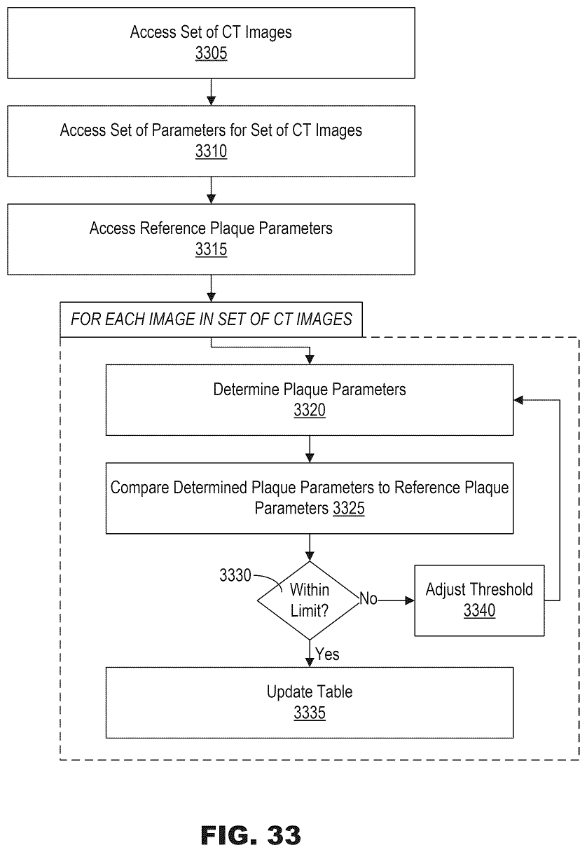

FIG. 33 is a flowchart that illustrates an example process for creating and/or updating a calcified plaque threshold table according to some embodiments. At operation 3305, a system can access a set of CT images. At operation 3310, the system can access CT scan parameter data for the set of CT images. In some embodiments, the system also accesses data such a scanner type, reconstruction method, patient parameters (e.g., weight, sex, body mass index, etc.). At operation 3315, the system can access reference calcified plaque parameters, for example as determined from invasive IVUS and/or OCT measurements. For each image in the set of CT images, the system can carry out operations 3320 through 3340. At operation 3320, the system can determine plaque parameters by analyzing the image. At operation 3325, the system can compare the determined plaque parameters to corresponding reference plaque parameters. At operation 3330, the system can determine if a difference between the determined plaque parameters and the reference plaque parameters is within a limit. If so, the system can update plaque calcification threshold table at operation 3335. If not, the system can adjust the plaque calcification threshold at 3340 and redetermine the plaque parameters. The process can continue until the determined parameters are within the limit of the reference parameters. It will be appreciated that there can be multiple parameters, and limits may be different for different parameters.

In some embodiments, updating the table at operation 3335 can include adding a new value to the table or updating an existing value in the table. In some embodiments, underlying data for determining the values in the table can be stored to enable updating of the table. As a simple example, if the only parameter is kV p, the system can track CT images with the same kVp and the calcified plaque thresholds determined for each of those images. Computing an updated calcified plaque threshold can include averaging a new determined threshold and previously determined thresholds.

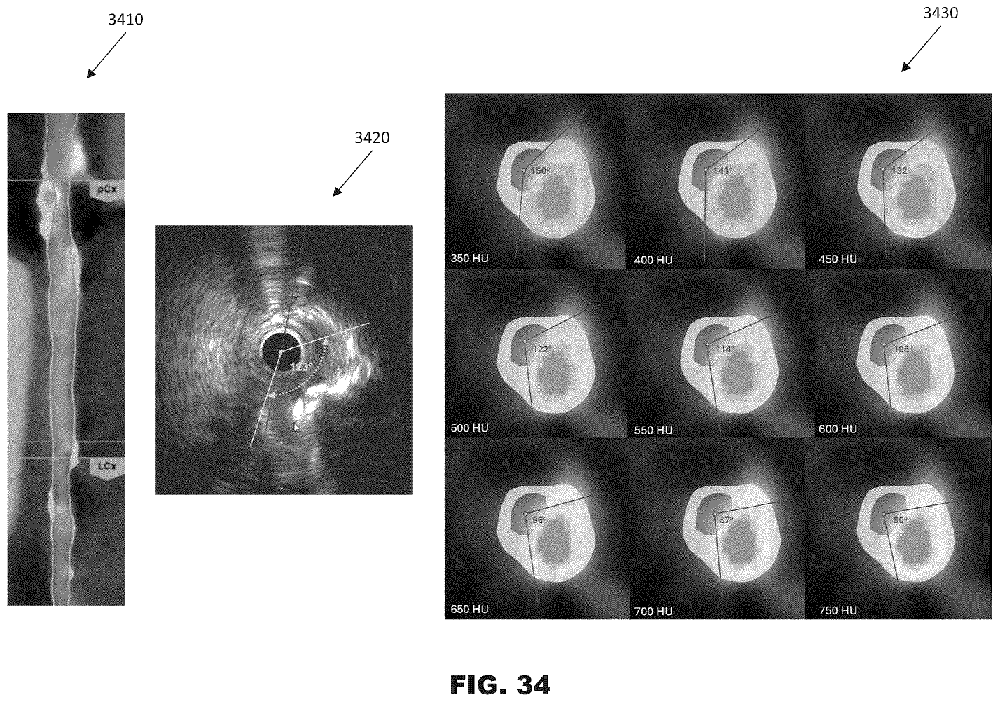

FIG. 34 is a drawing that illustrates calcified plaque determination using IVUS according to some embodiments. The straightened view 3410 shows a straightened view of (a portion of) a vessel, and the IVUS image 3420 shows an angle of contact between non-calcified plaque and a lumen. The IVUS image 3420 can be treated as a baseline or reference value for the angle of contact between non-calcified plaque and the lumen. Images 3430 are cross-sectional CT images with varying plaque thresholds. As shown in images 3430, the angle of contact varies significantly (e.g., from about 80 degrees to about 150 degrees) depending upon the calcified plaque threshold. in the example of FIG. 34, a calcified plaque threshold of 500 HU most closely matches the value determined via IVUS (e.g., 122 degrees vs. 123 degrees).

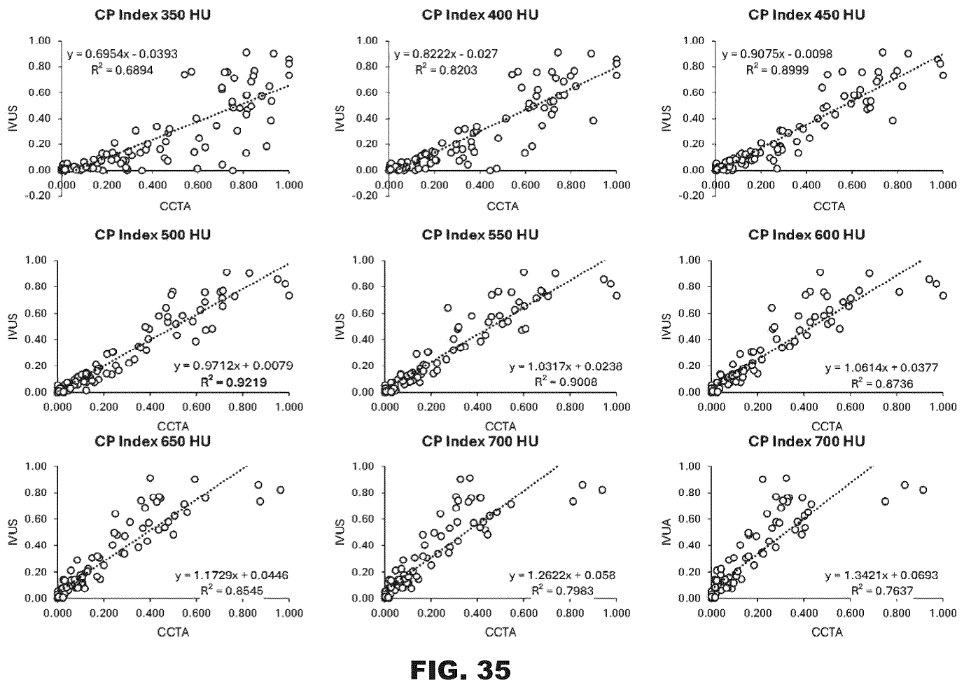

FIG. 35 is a diagram that illustrates example correlations between calcified plaque as determined by IVUS and CCTA for different calcified plaque thresholds. As shown in FIG. 35, the strongest correlation between IVUS and CCTA is obtained at a calcified plaque threshold of 500 HU.

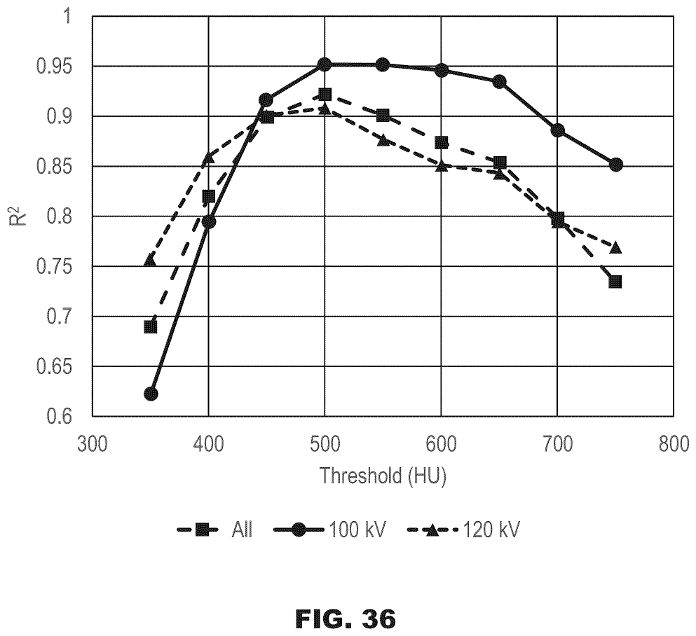

FIG. 36 is a plot that illustrates R² values at different calcified plaque thresholds for different peak kilovoltages (kVp) according to some embodiments. kVp can have a significant impact on a CCTA image. As shown in FIG. 36, the correlation of IVUS and CCTA is different at 100 kVp and 120 kV p. In the example of FIG. 36, 100 kVp exhibits a larger window of strong correlation than 120 kV p. Thus, for example, an image collected at 100 kVp may be someone less sensitive to the calcified plaque threshold than an image captured at 120 kV p.

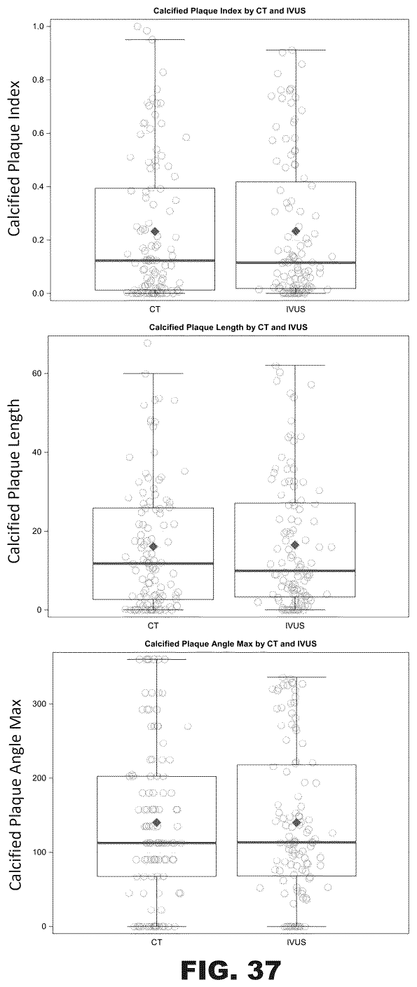

FIG. 37 illustrates box plots of calcified plaque index, calcified plaque length, and calcified plaque maximum angle as determined by CT and IVUS according to some embodiments. As shown in FIG. 37, similar results can be obtained using CT or IVUS, thus demonstrating that CT can be an effective non-invasive technique for determining calcified plaque parameters as compared to the invasive IVUS.

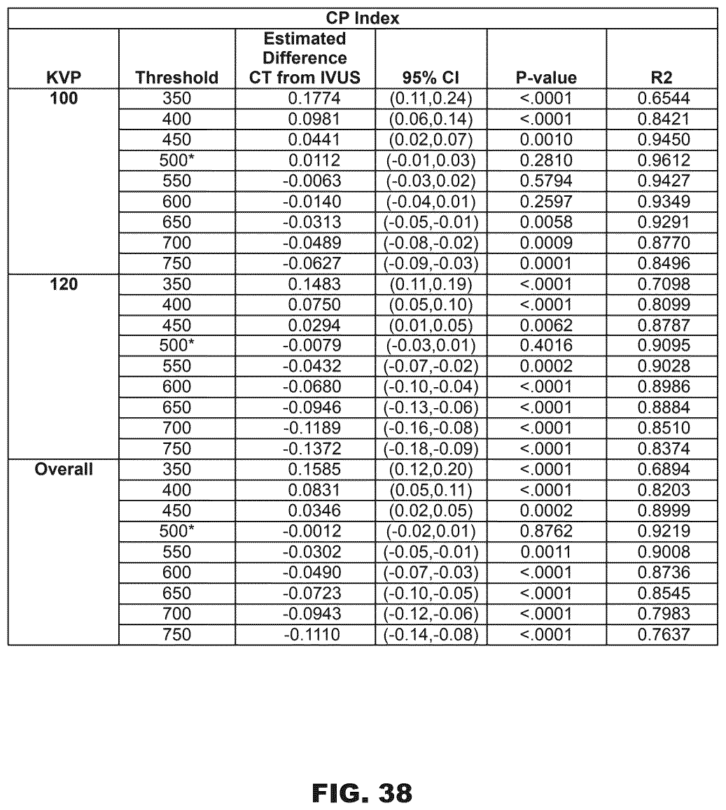

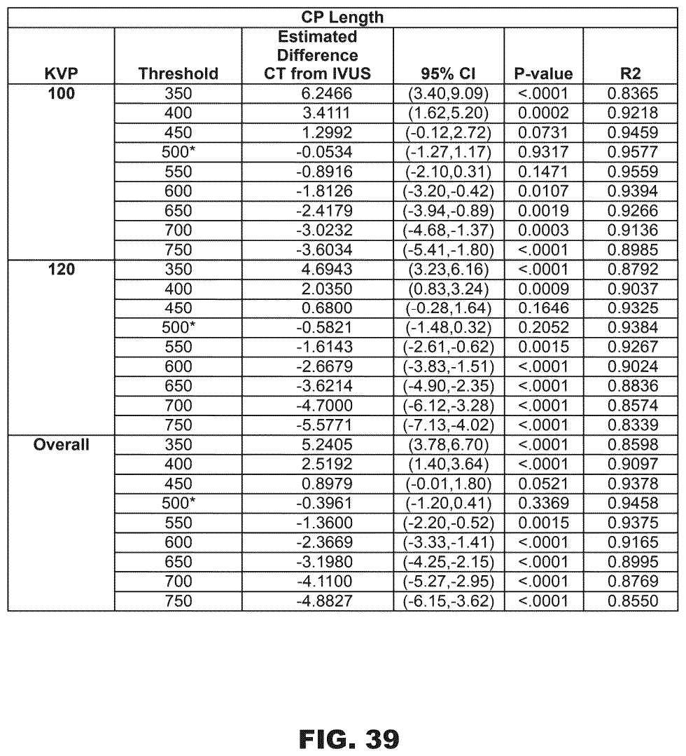

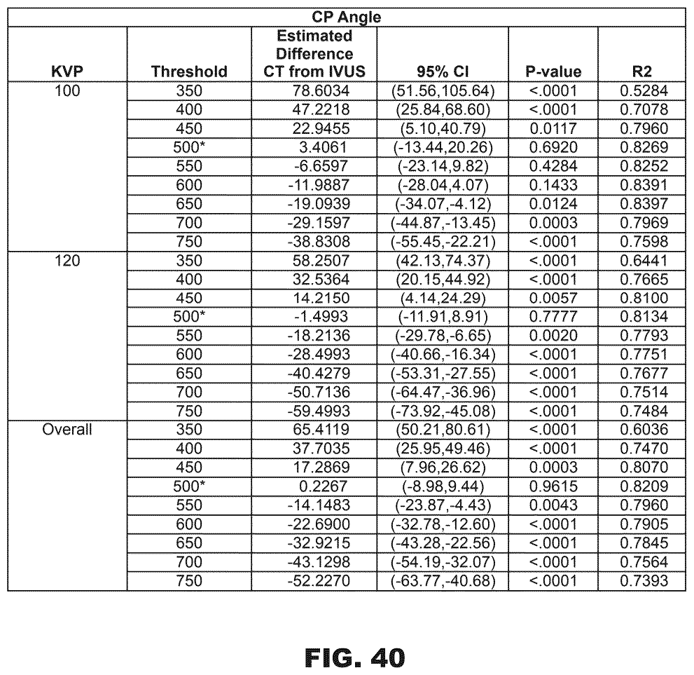

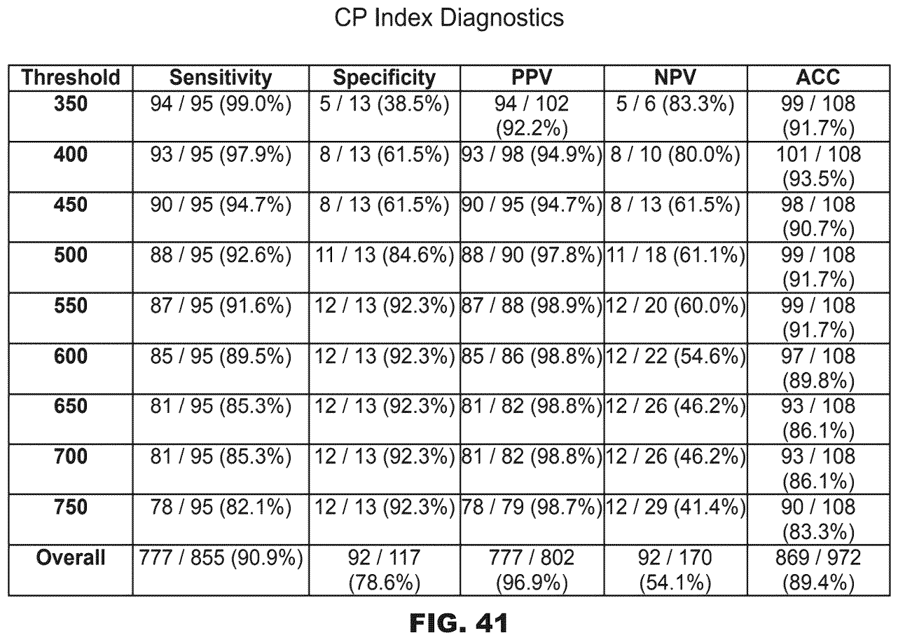

FIG. 38 is a table that compares calcified plaque index as determined by CT and IVUS according to some embodiments. FIG. 39 is a table that compares calcified plaque length as determined by CT and IVUS according to some embodiments. FIG. 40 is a table that shows examples of comparisons of calcified plaque angle as determined by CT and by IVUS according to some embodiments. FIG. 41 is a table that shows examples of sensitivity, specificity, positive predictive value (PPV), negative predictive value (NPV), and accuracy at various calcified plaque thresholds according to some embodiments.

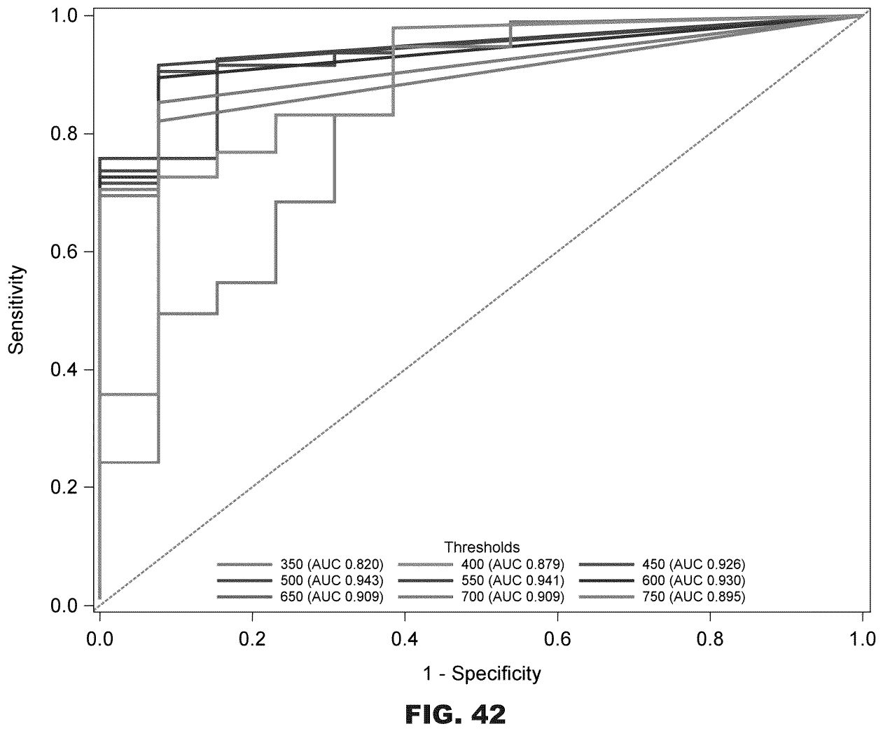

FIG. 42 is a receiver operating characteristic plot showing various calcified plaque thresholds in comparison with IVUS measurements according to some embodiments.

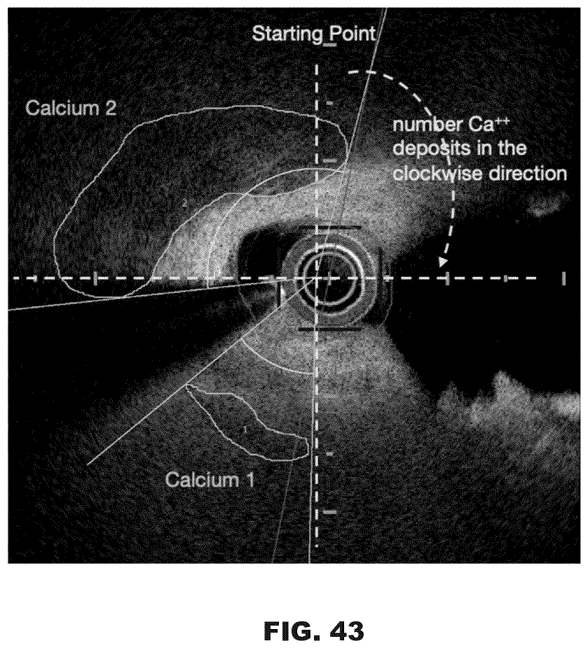

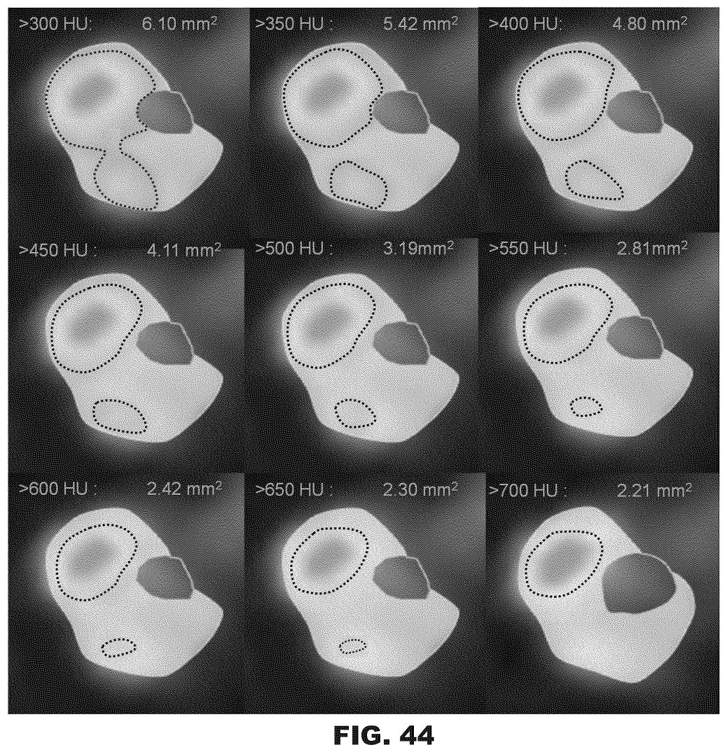

FIG. 43 shows an example OCT image with regions of calcium according to some embodiments. The total area of calcified plaques in the image is 2.38 mm². FIG. 44 is a drawing that illustrates calcified plaque (blue regions encircled in dashed lines) and non-calcified plaque at various calcified plaque thresholds according to some embodiments. As shown in FIG. 44, the total calcified plaque volume determined using CCTA varies with calcified plaque threshold, from 6.10 mm² at 300 HU to 2.21 mm² at 700 HU. As shown in FIG. 44, the best match with the OCT result was obtained at 600 HU. Some conventional approaches use a fixed threshold of 350 HU. In FIG. 44, 350 HU resulted in a calcified plaque area of 5.42 mm², corresponding to an overestimation of calcified plaque area by a factor of more than two.

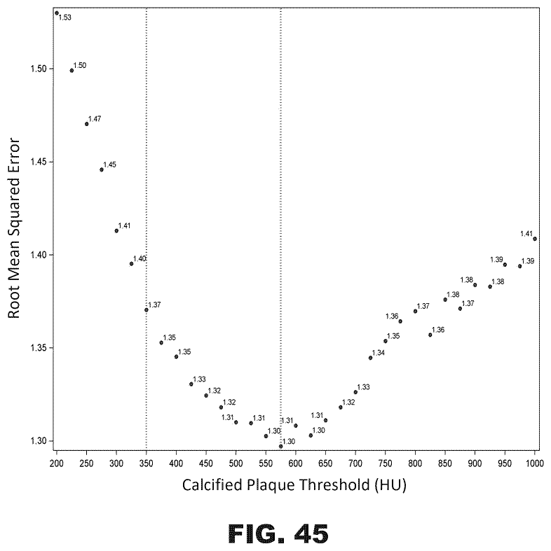

FIG. 45 is a plot that shows root mean squared error (RMSE) as a function of calcified plaque threshold according to some embodiments. As shown in FIG. 45, the lowest RMSE between OCT and CCTA was obtained at a calcified plaque threshold of between 550 HU and 650 HU.

While specific thresholds are discussed in the preceding discussion, it will be appreciated that these are merely examples, and the actual calcified plaque threshold that produces calcified plaque parameters that most closely match invasively determined reference values can depend on a wide variety of factors as described herein.

### Example Calcium Blooming Embodiments

Embodiment 1. A computer-implemented method for determining a desired calcified plaque density threshold for a coronary computed tomography angiography (CCTA) image of a subject to reduce calcium blooming effects for improved analysis of calcified plaque, the method comprising: accessing, by a computer system, the CCTA image of the subject, the CCTA image of the subject depicting one or more regions of one or more arteries of the subject, wherein the one or more regions comprise one or more regions of plaque; accessing, by the computer system, a set of scan parameter data associated with the CCTA image; determining, by the computer system based at least in part on the set of scan parameter data, a desired calcified plaque density threshold for the CCTA image to reduce calcium blooming for improved analysis of calcified plaque, wherein the desired calcified plaque threshold is determined using a machine learning model, wherein the machine learning model is trained by: accessing a set of sample CCTA images; accessing a set of sample invasively obtained medical images, wherein each of the CCTA images of the set of sample CCTA images corresponds to an invasively obtained medical image of the set of sample invasively obtained medical images, and wherein each of the CCTA images of the set of sample CCTA images is co-registered with its corresponding invasively obtained medical image of the set of sample invasively obtained medical images; accessing, for each CCTA image of the set of sample CCTA images, the set of scan parameters; analyzing, for each invasively obtained medical image of the set of sample invasively obtained medical images, to identify one or more regions of calcified plaque; and training a machine learning algorithm to determine a desired calcified plaque density threshold for reducing calcium blooming, wherein the machine learning model is trained using supervised learning to output a desired calcified plaque density threshold based at least in part on a correlation between the set of scan parameters and the one or more regions of calcified plaque; and determining, by the computer system, one or more calcified plaque parameters from the CCTA image using the desired calcified plaque threshold, wherein the computer system comprises a computer processor and a non-volatile computer-readable medium.
Embodiment 2. The computer-implemented method of embodiment 1, further comprising generating a modified CCTA image, wherein the modified CCTA image is adjusted such that an apparent effect of calcium blooming is reduced in the modified CCTA image relative to the CCTA image of the subject.
Embodiment 3. The computer-implemented method of embodiment 1 or 2, wherein the invasively obtained medical images comprise an optical coherence tomography (OCT) image or an intravascular ultrasound (IVUS) image.
Embodiment 4. The computer-implemented method of any of embodiments 1-3, wherein the one or more calcified plaque parameters comprises one or more of: a calcified plaque outer boundary, a calcified plaque area, a calcified plaque length, or a calcified plaque area.
Embodiment 5. The computer-implemented method of any of embodiments 1-4, wherein the scan parameter data comprises at least one of: a peak kilovoltage (kV p) or a current (mA).
Embodiment 6. The computer-implemented method of any of embodiments 1-5, wherein the calcified plaque threshold is about 500 HU.
Embodiment 7. The computer-implemented method of any of embodiments 1-4, wherein the calcified plaque threshold is between about 450 HU and about 550 HU.
Embodiment 8. The computer-implemented method of any of embodiments 1-4, wherein the calcified plaque threshold is between about 400 HU and about 600 HU.
Embodiment 9. The computer-implemented method of any of embodiments 1-4, wherein the calcified plaque threshold is between about 300 Hounsfield units and about 700 Hounsfield units.
Embodiment 10. The computer-implemented method of any of embodiments 1-4, wherein the calcified plaque threshold is between about 351 Hounsfield units and about 2500 Hounsfield units.
Embodiment 11. The computer-implemented method of any of embodiments 1-4, wherein the calcified plaque threshold is between about 500 Hounsfield units and about 600 Hounsfield units.
Embodiment 12. A computer-implemented method for determining a calcified plaque threshold for a medical image of a subject, the method comprising: accessing, by a computer system, the medical image of the subject, wherein the medical image comprises a computed tomography (CT) image; accessing, by the computer system, a set of scan parameter data associated with the medical image; determining, by the computer system based at least in part on the set of scan parameter data, a calcified plaque threshold for the medical image, wherein the calcified plaque threshold is determined using a machine learning algorithm, wherein the machine learning algorithm is trained by: accessing a set of sample CT images; accessing a set of scan parameter data, wherein the set of scan parameter data comprises scan parameter data for each CT image of the set of sample CT images; accessing a set of reference calcified plaque parameters, the set of reference calcified plaque parameters comprising reference calcified plaque parameters corresponding to each CT image of the set of sample CT images; providing the set of CT images, the set of scan parameter data, and the reference calcified plaque parameters to a machine learning model; determining, using the machine learning model, the calcified plaque threshold; determining, using the calcified plaque threshold, a set of calcified plaque parameters; and adjusting, based at least in part on a comparison of the set of calcified plaque parameters and the set of reference calcified plaque parameters, one or more weights of the machine learning model; identifying, by the computer system, a region of plaque in the medical image; and determining, by the computer system, one or more plaque parameters for the region of plaque in the medical image, wherein the computer system comprises a process and a non-transitory computer-readable storage medium.
Embodiment 13. The computer-implemented method of embodiment 12, wherein the reference calcified plaque parameters are determined by: accessing the set of CT images; accessing a set of corresponding invasive images, wherein invasive image of the set of corresponding invasive images corresponds an image of the set of CT images; co-registering each image of the set of CT images with the corresponding invasive image of the set of corresponding invasive images; determining, for each image, corresponding calcified plaque parameters, wherein the corresponding calcified plaque parameters are determined by accessing the corresponding invasive image.
Embodiment 14. The computer-implemented method of embodiment 13, wherein the set of corresponding invasive images comprises intravascular ultrasound (IVUS) images. or optical coherence tomography (OCT) images.
Embodiment 15. The computer-implemented method of any of embodiment 12-14, wherein the calcified plaque threshold is about 500 HU.
Embodiment 16. The computer-implemented method of any of embodiments 12-14, wherein the calcified plaque threshold is between about 450 HU and about 550 HU.
Embodiment 17. The computer-implemented method of any of embodiments 12-14, wherein the calcified plaque threshold is between about 400 HU and about 600 HU.
Embodiment 18. The computer-implemented method of any of embodiments 12-14, wherein the calcified plaque threshold is between about 300 Hounsfield units and about 700 Hounsfield units.
Embodiment 19. The computer-implemented method any of embodiments 12-14, wherein the calcified plaque threshold is between about 351 Hounsfield units and about 2500 Hounsfield units.
Embodiment 20. The computer-implemented method of any of embodiments 12-14, wherein the calcified plaque threshold is between about 500 Hounsfield units and about 600 Hounsfield units.
Embodiment 21. The computer-implemented method of any of embodiments 12-14, wherein the set of scan parameter data comprises peak kilovoltage.
Embodiment 22. The computer-implemented method of any of embodiments 12-21, wherein the plaque parameters comprise one or more of total plaque volume, calcified plaque volume, non-calcified plaque volume, total plaque area, calcified plaque area, non-calcified plaque area, total plaque length, calcified plaque length, or non-calcified plaque length.
Embodiment 23. The computer-implemented method of any of embodiments 12-22, wherein the reference plaque parameters comprise at least one of calcified plaque index, calcified plaque length, or calcified plaque maximum angle.
Embodiment 24. The computer-implemented method of any of embodiments 12-23, wherein the medical image of the subject depicts a plurality of vessels.
Embodiment 25. The computer-implemented method of embodiment 24, wherein the plurality of vessels comprises one or more coronary arteries.
Embodiment 26. The computer-implemented method of embodiment 25, wherein the one or more coronary arteries comprise one or more of left main (LM), ramus intermedius (RI), left anterior descending (LA D), diagonal 1 (D1), diagonal 2 (D2), left circumflex (Cx), obtuse marginal 1 (OM1), obtuse marginal 2 (OM2), left posterior descending artery (L-PDA), left posterolateral branch (L-PLB), right coronary artery (RCA), right posterior descending artery (R-PDA), or right posterolateral branch (R-PLB).
Embodiment 27. A system for determining a calcified plaque threshold, the system comprising: at least one processor; and a computer-readable, non-transitory medium storing instructions which, when executed by the at least one processor, cause the system to: access a medical image of a subject, wherein the medical image comprises a computed tomography (CT) image; access a set of scan parameter data associated with the medical image; determine, based at least in part on the set of scan parameter data, a calcified plaque threshold for the medical image, wherein determining the calcified plaque threshold is performed using a machine learning model, wherein the machine learning model is trained by: accessing a set of CT images; accessing a set of scan parameter data, wherein the set of scan parameter data comprises scan parameter data for each CT image of the set of CT images; accessing a set of reference calcified plaque parameters, the set of reference calcified plaque parameters comprising reference calcified plaque parameters corresponding to each of CT image of the set of CT images; providing the set of CT images, the set of scan parameter data, and the reference calcified plaque parameters to a machine learning model; determining, using the machine learning model, the calcified plaque threshold; determining, using the calcified plaque threshold, a set of calcified plaque parameters; and adjusting, based at least in part on a comparison of the set of calcified plaque parameters and the set of reference calcified plaque parameters, one or more weights of the machine learning model; identifying, by the computer system, a region of plaque in the medical image; and determining, by the computer system, one or more plaque parameters for the region of plaque in the medical image.
Embodiment 28. The system of embodiment 27, wherein the reference calcified plaque parameters are determined by: accessing the set of CT images; accessing a set of corresponding invasive images, wherein invasive image of the set of corresponding invasive images corresponds an image of the set of CT images; co-registering each image of the set of CT images with the corresponding invasive image of the set of corresponding invasive images; determining, for each image, corresponding calcified plaque parameters, wherein the corresponding calcified plaque parameters are determined by accessing the corresponding invasive image.
Embodiment 29. The system of embodiment 28, wherein the set of corresponding invasive images comprises intravascular ultrasound (IVUS) images or optical coherence tomography (OCT) images.
Embodiment 30. The system of any of embodiments 27-29, wherein the calcified plaque threshold is about 500 HU.
Embodiment 31. The system of any of embodiments 27-29, wherein the calcified plaque threshold is between about 450 HU and about 550 HU.
Embodiment 32. The system of any of embodiments 27-29, wherein the calcified plaque threshold is between about 400 HU and about 600 HU.
Embodiment 33. The system of any of embodiments 27-29, wherein the calcified plaque threshold is between about 300 Hounsfield units and about 700 Hounsfield units.
Embodiment 34. The system of any of embodiments 27-29, wherein the calcified plaque threshold is between about 351 Hounsfield units and about 2500 Hounsfield units.
Embodiment 35. The system of any of embodiments 27-29, wherein the calcified plaque threshold is between about 500 Hounsfield units and about 600 Hounsfield units.
Embodiment 36. The system of any of embodiments 27-35, wherein the plaque parameters comprise one or more of total plaque volume, calcified plaque volume, non-calcified plaque volume, total plaque area, calcified plaque area, non-calcified plaque area, total plaque length, calcified plaque length, or non-calcified plaque length.
Embodiment 37. The system of any of embodiments 27-36 wherein the reference plaque parameters comprise at least one of calcified plaque index, calcified plaque length, or calcified plaque maximum angle.
Embodiment 38. The system of any of embodiments 27-37, wherein the medical image of the subject depicts a plurality of vessels.
Embodiment 39. The system of embodiment 38, wherein the plurality of vessels comprises one or more coronary arteries.
Embodiment 40. The system of embodiment 39, wherein the one or more coronary arteries comprise one or more of left main (LM), ramus intermedius (RI), left anterior descending (LAD), diagonal 1 (D1), diagonal 2 (D2), left circumflex (Cx), obtuse marginal 1 (OM1), obtuse marginal 2 (OM2), left posterior descending artery (L-PDA), left posterolateral branch (L-PLB), right coronary artery (RCA), right posterior descending artery (R-PDA), or right posterolateral branch (R-PLB).

### Thin Cap Fibroatheroma Identification

In the present disclosure, approaches for detecting thin cap fibroatheroma (TCFA) using coronary computed tomography angiography (CCTA) imaging are disclosed. The approaches described herein can enable non-invasive detection and/or evaluation of TCFA. In some embodiments, the approaches herein enable characterization of low attenuation plaque that is more accurate and/or in closer agreement with low attenuation plaque characterization done using invasive imaging methods such as optical coherence tomography (OCT).

Fibroatheroma is a type of plaque that can present a significant risk of adverse health events, but some fibroatheromas are more likely to result in adverse health events than others. Fibroatheromas can be characterized by the presence of a fibrous cap that forms a boundary between the core of the fibroatheroma and a lumen. The thickness of the cap can be a strong indicator of the likelihood that a fibroatheroma will rupture. When a fibroatheroma ruptures, the thrombogenic core of the fibroatheroma can be released into the lumen, resulting in thrombosis. Studies have shown that plaques most likely to rupture can be characterized by the presence of a thin fibrous cap that is less than 65 micrometers thick. Fibroatheromas with such thin caps can be categorized as thin cap fibroatheromas (TCFAs).

Accurately imaging such a thin cap can be difficult. For example, intravascular ultrasound virtual histology (IVUS-VH) can have an axial resolution of about 200 micrometers, limiting its ability to identify TCFA. CCTA also lacks the ability to reliably image such thin caps. In many cases, optical coherence tomography (OCT) is used to identify and quantify TCFAs.

According to some implementations herein, TCFA can be identified without necessarily imaging the cap itself and/or without having a high accuracy measure of the cap's thickness. For example, the presence of low attenuation non-calcified plaque near a lumen can indicate the presence of TCFA. However, CCTA can struggle to identify low-attenuation plaques, presenting challenges for using CCTA to identify TCFA. Invasive procedures such as optical coherence tomography (OCT) may be more reliable but can be complex and pose some patient risk.

Determining plaque density and/or calcification can be accomplished using CCTA images according to some implementations as described herein. For example, contrast (e.g., as measured in Hounsfield Units (HU)) can be indicative of density and/or calcification. The apparent size, density, and so forth of a plaque can be influenced by the contrast of the nearby lumen, the peak kilovoltage (kVp) of an x-ray source used to collect the CCTA image, the current (mA) used, spot size, collimators, and/or other scan parameters and/or equipment configurations. CCTA images can be influenced by the scanner type, reconstruction method used, lumen enhancement (e.g., contrast agent) and so forth. While settings and scanner configuration can have a significant influence on CCTA images, patient factors can also play an important role. For example, a patient's sex, age, body mass index, and/or other demographic and/or physiologic parameters can significantly impact a CCTA image. For example, obesity can lead to higher levels of image noise due to the greater amount of tissue that X-rays must penetrate. Variations in chest anatomy can complicate image acquisition and image. Physiological features such as heart rate, breath-holding ability, cardiac output and blood flow, and so forth can affect CCTA images. For example, high heart rate can lead to motion artifacts. Arrhythmias can complicate image acquisition and reconstruction as it can be difficult to synchronize image acquisition with the patient's heartbeat. Poor breath-holding ability can result in motion artifacts. High cardiac output can affect the timing of contrast administration and image acquisition, which can result in poor contrast of the coronary arteries.

According to some implementations, CCTA images can be normalized based on lumen contrast (which can be measured in Hounsfield units (HU)) and kVp. As a result, low-attenuation plaque (LA P) can be differentiated from other non-calcified plaque and/or from calcified plaque. In some embodiments, other factors can be taken into account additionally or alternatively, such as current, reconstruction algorithm, patient anatomy and/or physiology, and so forth.

According to some implementations, TCFA can be determined by the presence of LAP at or near a boundary of a plaque, for example at the interface between the plaque and the lumen.

Plaque types can be classified in CCTA images based on their attenuation as measured in HU. Typically, lower HU values can indicate less calcification and/or other differences in plaques. While CCTA alone can provide relative information about plaques (e.g., more calcification or less calcification), it can be important to obtain a more absolute measure of plaque properties so that plaques are accurately classified, and treatment approaches can be determined based on reliable information. By setting appropriate thresholds for different types of plaque/different plaque densities, CCTA can be used to detect the presence of and/or to quantify different types of plaque.

In some cases, CCTA imaging may not be able to resolve a thin cap, but it may be able to differentiate between LAP and other non-calcified plaques. In some implementations, a threshold between LAP and other non-calcified plaque for CCTA images can be determined based on OCT images. Advantageously, it has been found by the inventors that proximity and/or apparent contact of LAP with a lumen as observed in CCTA images can be used to identify the presence of TCFA in patients.

In some implementations, a set of CCTA images and a set of OCT images can be co-registered, and a threshold (in HU) cutoff between LAP and other non-calcified plaque can be adjusted for the CCTA images so that the CCTA images more closely reflect the extent of LAP as determined using OCT. In some implementations, only cross-sectional images may be used. Cross-sectional images can show the proximity of LAP to the boundary between the plaque and the lumen. In some implementations, three dimensional images can be used, which can provide additional and/or different insight into the extent of LAP. In some implementations, analysis is not limited to axial images but can include, additionally or alternatively, sagittal, coronal, and/or other oblique images.

To identify TCFA, it may not be necessary to know the precise size and shape of the LA P. For example, it can be sufficient to determine that the LAP extends to or close to the surface of the plaque. In some implementations, a set of OCT images can be divided into groups, with one group comprising images where TCFA is present and the other comprising images where TCFA is not present. In some implementations, thresholds for differentiating between LAP and other non-calcified plaque can be adjusted so that CCTA images can reliably be categorized as showing TCFA or not showing TCFA, e.g., as determined by comparison with OCT images. In some implementations, the presence of TCFA can be determined based at least in part on one or more additional factors, such as degrees of contact between the LAP and the lumen. For example, if a degree of contact is small or contact only appears in a single slice of a CCTA image, this can indicate either that that there is no TCFA (e.g., the contact may simply be an image artifact) or the TCFA may be present but of low or mild severity. In contrast, if a degree of contact is relatively large, this can more strongly indicate the presence of TCFA. The contact between the LAP and lumen can be indicative of a likelihood that a TCFA will rupture. Thus, degree of contact can be used in determining a relative vulnerability of plaque.

In some implementations, an apparent thickness of a cap is determined from CCTA images. As described herein, such thickness may not be accurate, but it is not necessary in some implementations to have an accurate thickness measurement or accurate information about the size and shape of LAP in order to detect TCFA. Rather, it can be sufficient that there is a clear separation between apparent cap thicknesses in CCTA images where TCFA is present and CCTA images where TCFA is not present, and/or it can be sufficient that the location of LAP (e.g., relative to the boundary between the plaque and the lumen) is determined well enough to differentiate between TCFA and other plaques.

In some implementations, a single image or slice is analyzed to determine the presence of TCFA. However, such an approach may produce erroneous results in some circumstances. For example, a single image may show signs of TCFA because of image artifacts, imaging parameters, anatomy and physiology of the patient, the processing algorithm used to process the image data, the particular thresholds used to distinguish LAP from other non-calcified plaque, and so forth. Accordingly, in some implementations, an entire volume or multiple slices can be used to identify TCFA. The multiple slices can be slices along a single axis or along more than one axis.

In some embodiments, various criteria can be set for determining that TCFA is present based on CT analysis. For example, TCFA can be identified when TCFA is present in at least a threshold number of consecutive slices (e.g., 2 slices, 3 slices, 4 slices, 5 slices, etc.). The threshold number of slices can depend on various factors such as the slice thickness, distance between slices (e.g., center to center distance), and so forth. In some embodiments, other criteria can be used additionally or alternatively to identify TCFA and/or to determine a risk level associated with TCFA. For example, if the necrotic core (LA P) area is more than a threshold fraction of the total plaque area, this can indicate the presence of TCFA. Higher ratios of necrotic core to total plaque area can indicate increased vulnerability. Other measures can be used additionally or alternatively, such as the ratio of the volume of necrotic core to total plaque volume. In some embodiments, vulnerability can be based at least in part on plaque burden. For example, the percentage plaque burden being at or over a threshold value can indicate increased vulnerability.

In some implementations, a static value can be used to define a LAP threshold. This can work well in many cases, but has certain limitations as it may not be applicable to all scanning conditions, all patient anatomies, all patient physiologies, and so forth. In some implementations, a lookup table can be used to determine an LAP threshold. The lookup table can include one or more parameters. For example, relatively simple implementation of a lookup table can indicate the LAP threshold in relation to a single variable such as peak kilovoltage. M ore complex lookup tables can also be implemented. A more complex lookup table can account for, for example, additional scan parameters, reconstruction algorithms, patient anatomy, patient physiology, and so forth. However, such a lookup table can be large and unwieldly, and the time required to look up a LAP threshold in such a table can be significant. Moreover, the table may not have an entry for every combination of scan parameter, reconstruction algorithm, patient anatomy parameter, patient physiology parameter, etc. Thus, in some cases, it can be necessary to interpolate or otherwise estimate a LAP threshold based on information that is present in the table. While this can work in some circumstances, such an approach can produce poor results when there are complex dependencies, interrelationships between parameters, and so forth. In some cases, a lookup table may include only a limited number of parameters, for example, only the parameters that most influence the LAP threshold. However, selecting such parameters can be challenging and can fail to account for the complex influence different other parameters or combinations of parameters can have on the appropriate LA P threshold. Additionally, it can be difficult to add new parameters to a lookup table.

While there are several downsides associated with using a lookup table, a lookup table may nonetheless be effective in many cases and can be desirable because it provides an explainable source for how a LAP threshold was determined (e.g., which parameters were considered in determining the LAP threshold). Accordingly, some implementations use a lookup table.

In some implementations, a machine learning model can be trained to determine a LA P threshold for an image. For example, as described in more detail herein with respect to the figures, a machine learning model can be trained using supervised learning to determine a LAP threshold appropriate for a received CCTA image. The model can be trained using, for example, supervised learning, in which reference values (e.g., as determined by OCT) are supplied as the target outputs and CCTA images are used as the inputs.

In some implementations, a LAP threshold can be 0 HU or about 0 HU, 1 HU or about 1 HU, 2 HU or about 2 HU, 3 HU or about 3 HU, 4 HU or about 4 HU, 5 HU or about 5 HU, 6 HU or about 6 HU, 7 HU or about 7 HU, 8 HU or about 8 HU, 9 HU or about 9 HU, 10 HU or about 10 HU, 11 HU or about 11 HU, 12 HU or about 12 HU, 13 HU or about 13 HU, 14 HU or about 14 HU, 15 HU or about 15 HU, 16 HU or about 16 HU, 17 HU or about 17 HU, 18 HU or about 18 HU, 19 HU or about 19 HU, 20 HU or about 20 HU, 21 HU or about 21 HU, 22 HU or about 22 HU, 23 HU or about 23 HU, 24 HU or about 24 HU, 25 HU or about 25 HU, 26 HU or about 26 HU, 27 HU or about 27 HU, 28 HU or about 28 HU, 29 HU or about 29 HU, 30 HU or about 30 HU, 31 HU or about 31 HU, 32 HU or about 32 HU, 33 HU or about 33 HU, 34 HU or about 34 HU, 35 HU or about 35 HU, 36 HU or about 36 HU, 37 HU or about 37 HU, 38 HU or about 38 HU, 39 HU or about 39 HU, 40 HU or about 40 HU, 41 HU or about 41 HU, 42 HU or about 42 HU, 43 HU or about 43 HU, 44 HU or about 44 HU, 45 HU or about 45 HU, 46 HU or about 46 HU, 47 HU or about 47 HU, 48 HU or about 48 HU, 49 HU or about 49 HU, 50 HU or about 50 HU, 51 HU or about 51 HU, 52 HU or about 52 HU, 53 HU or about 53 HU, 54 HU or about 54 HU, 55 HU or about 55 HU, 56 HU or about 56 HU, 57 HU or about 57 HU, 58 HU or about 58 HU, 59 HU or about 59 HU, 60 HU or about 60 HU, 61 HU or about 61 HU, 62 HU or about 62 HU, 63 HU or about 63 HU, 64 HU or about 64 HU, 65 HU or about 65 HU, 66 HU or about 66 HU, 67 HU or about 67 HU, 68 HU or about 68 HU, 69 HU or about 69 HU, 70 HU or about 70 HU, 71 HU or about 71 HU, 72 HU or about 72 HU, 73 HU or about 73 HU, 74 HU or about 74 HU, or 75 HU or about 75 HU. In some implementations, the LA P threshold can be any number between these numbers. In some implementations, the LAP threshold can be from about 0 HU to about 35 HU, for example from about 20 HU to about 35 HU.

While the preceding and following discussion focus on determining LAP thresholds, it will be appreciated that the techniques described herein are not strictly limited to determining thresholds for LAP. For example, the techniques herein can be readily adapted to determining non-calcified plaque thresholds and/or calcified plaque thresholds.

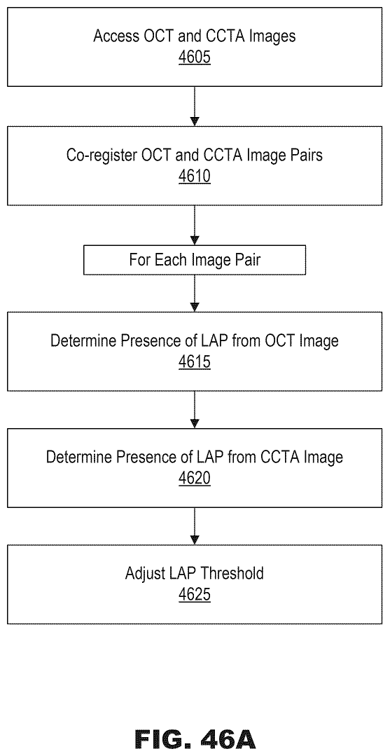

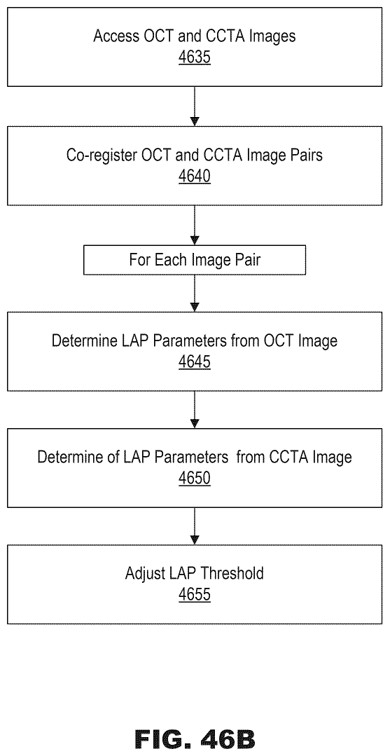

FIG. 46A is a flowchart that shows an example process for determining a threshold HU value for differentiating between LAP and other non-calcified plaque according to some implementations. The process shown in FIG. 46A can be performed by a computer system. The process of FIG. 46A is directed to identifying the presence of LA P.

At operation 4605, the system can receive a set of CCTA images and a set of OCT images. At operation 4610, the system can co-register the CCTA images and OCT images. For example, for each CCTA image, there can be a corresponding OCT image, and the CCTA image and OCT image can be co-registered. Operations 4615-725 can be carried out for each image pair. At operation 4615, the system can determine the presence of LAP in an OCT image. At operation 4620, the system can determine the presence of LAP from a corresponding CCTA, for example using a LAP threshold value. At operation 4625, the system can adjust the LAP threshold value such that the determined presence of LA P in the CCTA image matches the determined presence of LAP in the OCT images. If the presence of LAP in the CCTA image matches the presence of LAP in the OCT image, no adjustment to the LAP threshold may be made. The process can continue for each image pair, such that the LA P threshold is updated as new image pairs are processed. In some embodiments, the LA P threshold can be a plurality of thresholds. For example, each CCTA image can have one or more parameters (e.g., scan parameters) associated therewith, and LAP thresholds can be determined for various combinations of parameters.

While FIG. 46A illustrates a process for adjusting LAP thresholds based on a binary (e.g., yes/no) determination of the presence of LAP, it will be appreciated that the resulting LAP value can be more generally applicable. For example, the resulting LAP value can be used in more complex processes that involve, for example, determining the length, area, volume, geometry, etc., of LAP.

FIG. 46B is a flowchart that illustrates an example process for determining a LAP threshold according to some implementations. The process illustrated in FIG. 46B is similar to that shown in FIG. 46A, except that LAP parameters are determined.

At operation 4635, a system can access OCT and CCTA images. The images can be image pairs, with each OCT image corresponding to a CCTA image. At operation 4640, the system can co-register the OCT and CCTA image pairs. For each image pair, the system can perform operations 4645-755. At operation 4645, the system can determine LAP parameters (e.g., area, length, volume, abutment with a lumen, etc.) from the OCT image. At operation 4650, the system can determine LA P parameters from the CCTA image. At operation 4655, the system can adjust a LAP threshold, for example based on a mismatch between the LAP parameters determined from the OCT image and the LAP parameters determined from CCTA image.

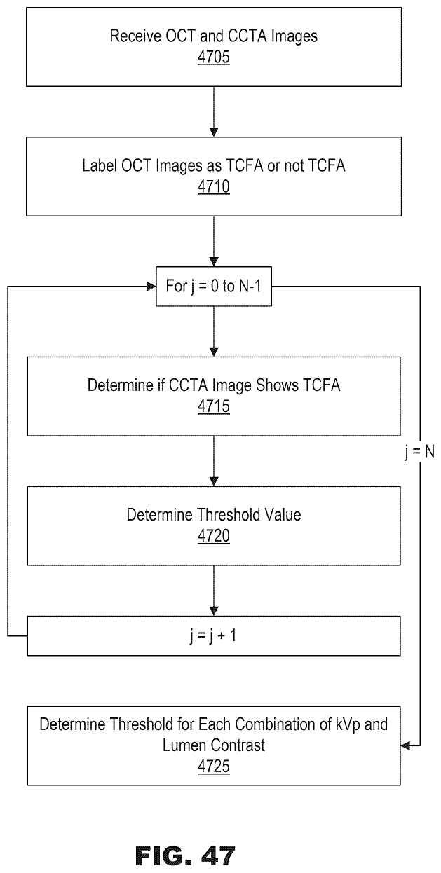

FIG. 47 is a flowchart that illustrates an example process for determining a threshold HU value for identifying TCFA according to some implementations. The process shown in FIG. 47 can be performed by a computer system.

At operation 4705, the system can receive a set of OCT images and a set of CCTA images. At operation 4710, the system can label the OCT images as TCFA or not TCFA. In some implementations, the system can analyze each OCT image to determine if it shows signs of TCFA or not. In some implementations, the OCT images can be pre-labeled as showing TCFA or not. Each OCT image can correspond to a CCTA image. For each CCTA image, the system can, at operation 4715, determine if the image shows TCFA, for example by determining whether or not LAP in the image extends to the boundary between the core of a plaque in the image and a lumen in the image. The system can compare this result to the label for the corresponding OCT image, and can, at operation 4720, determine a threshold value. For example, the system can iteratively modify the threshold value. In some implementations, the system can adjust the threshold value just until the analysis gives the incorrect result. In some implementations, the iterative adjustment can be, for example, 47 HU or about 47 HU, 20 HU or about 20 HU, 50 HU or about 50 HU, or more or less, or any value between these values. In this way, assuming a roughly equal distribution of TCFA and non-TCFA images, the system can determine a range of threshold values for identifying TCFA. In some implementations, equal or roughly equal may not be required. For example, results can be weighted such that an overall threshold value for a kVp and lumen contrast can be determined. At operation 4725, the system can determine a threshold value for each combination of kVp and Lumen contrast. In some implementations, Lumen contrast can be binned, for example in groups of 10 HU, 20 HU, 30 HU, 40 HU, 50 HU, 100 HU, or any value between these values, or more or less. Binning can ensure that each combination of kVp and lumen contrast has a sufficient number of samples to determine a reliable threshold value.

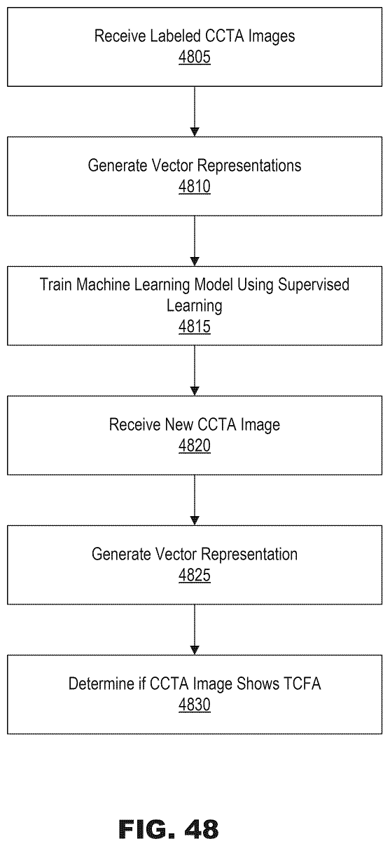

In some implementations, a machine learning model can be trained to identify TCFA in CCTA images. The machine learning model can be trained using supervised learning as described herein. FIG. 48 is a flow chart that illustrates an example process for identifying TCFA in CCTA images using machine learning according to some implementations. The process of FIG. 48 can be carried out by a computer system. In FIG. 48, training and deployment are illustrated as a single process. However, it will be understood that training and deployment can be implemented as separate processes that can be carried out on the same computer system or different computer systems.

At operation 4805, the system can receive a plurality of labeled CCTA images. The labels can indicate whether or not the CCTA image shows TCFA as well as the kVp used to capture the CCTA image. In some implementations, the labels can include a lumen contrast, although such information may not be included in some other implementations. At operation 4810, the system can generate vector representations of the CCTA images. In some implementations, the vector representations can include representations of one or more labels or parts of one or more labels. For example, the vector representation of an image can encode image data as well as the kVp used to capture the image. At operation 4815, the system can train a machine learning model using supervised learning. For example, a “TCFA present” label can indicate whether or not an image shows TCFA, and the model can be trained using supervised learning where “TCFA present” is the desired output.

After training, the model can be deployed. During deployment, the model can receive new CCTA images for which the presence of TCFA is unknown. The presence of TCFA can be determined by the model. At operation 4820, the system can receive a new CCTA image. At operation 4825, the system can generate a vector representation of the received CCTA image. The vector representation can include, in some implementations, a kV p value used to capture the CCTA image. At operation 4830, the system can, using the machine learning model trained at operation 4815, determine if the received CCTA image shows TCFA.

In the description of FIGS. 46A, 46B, 47, and 48, it is assumed that identification of TCFA is agnostic to the equipment used to capture images. However, in some cases, that may not be the case. For example, in some implementations, the x-ray source, x-ray detector, geometry (e.g., distance from source to detector), collimator used, and so forth can impact the resulting image. In some implementations, the total radiation dose to which a patient is exposed can impact the resulting image. In some cases, patient anatomy, physiology, or both can impact the resulting image.

While in many cases, consideration of lumen contrast and kVp can be used to account for the impact of such variations in image capture, in some cases, including some or all of the information above may result in improved accuracy. The processes described above can be readily modified to include additional/or different parameters, e.g., additional and/or additional parameters can be input into the machine learning model and/or included in the generated vector representations.

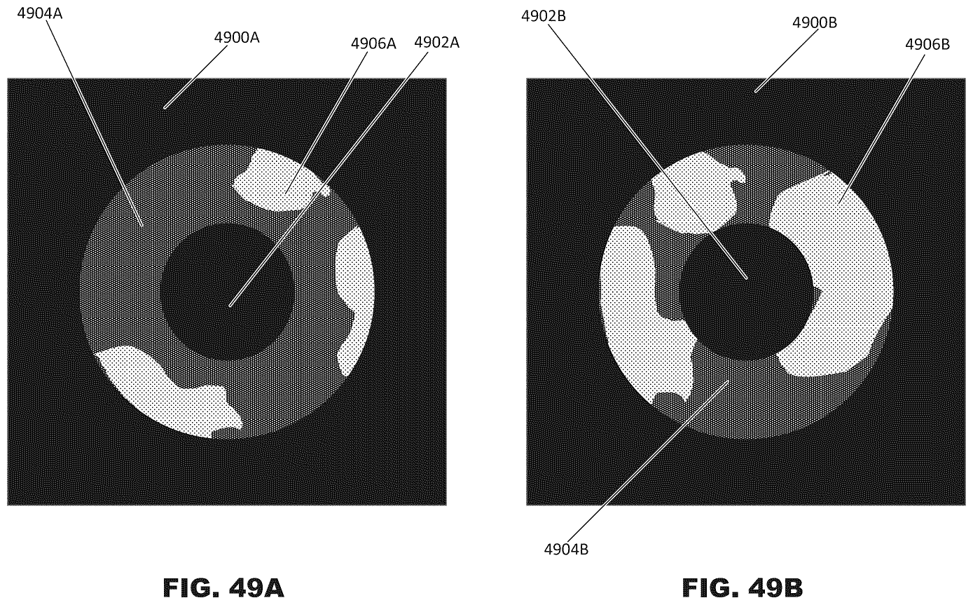

FIGS. 49A and 49B schematically illustrate examples of CCTA images that do not show TCFA (FIG. 49A) and do show TCFA (FIG. 49B). In the CCTA image 4900A, the lumen 4902A is surrounded by a plaque 4904A. In some regions, a low attenuation plaque 4906A is present. However, the low attenuation plaque 4906A does not extend to the interface between the lumen and the plaque. In some implementations, a system that implements one or more of the approaches described herein can identify the CCTA image 4900A as not showing TCFA. In the CCTA image 4900B, the plaque 4904B includes low attenuation plaque 4906B. In FIG. 49B, the low attenuation plaque 4906B extends to the boundary between the plaque and the lumen 4902B. In some implementations, a system that implements one or more of the approaches described herein can identify the CCTA image 4900B as showing the presence of TCFA.

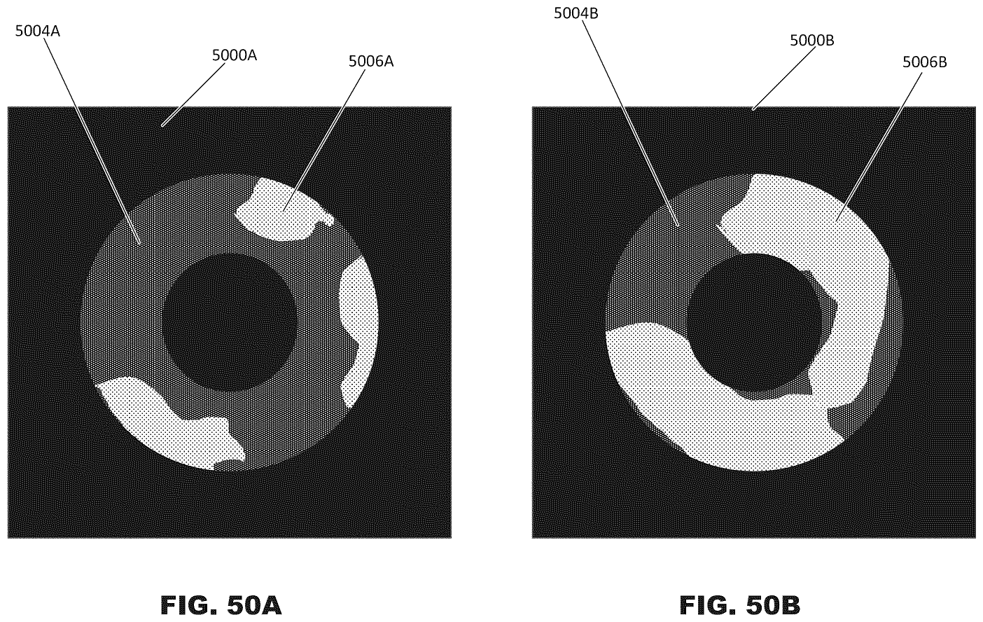

FIGS. 50A and 50B schematically illustrate an example of a CCTA image with different cutoff thresholds for LAP. In the CCTA image 5000A, a lower contrast cutoff is used to differentiate between LAP 5006A and non-LAP 5004A (which can include non-calcified and/or calcified plaque). The CCTA image 5000B schematically illustrates the 5000A, but with a different threshold for separating the LAP 5006B from other plaque 5004B. As shown in FIGS. 50A and 50B, the determination of whether or not TCFA is present (e.g., whether or not low attention plaque reaches the boundary between the plaque and a lumen) can change depending upon the selected threshold.

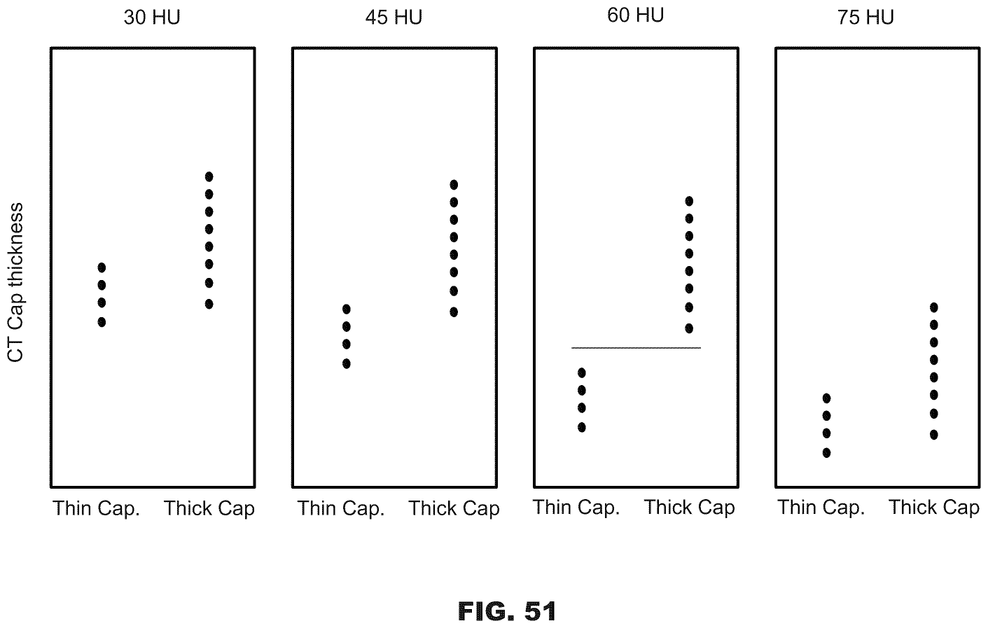

FIG. 51 illustrates an example of the separation between thin cap and thick cap fibroatheromas at different Hounsfield unit thresholds according to some implementations. In FIG. 51, the apparent cap thickness using CT can fail to distinguish between thin caps and thick caps, depending upon the chosen threshold. For example, at a threshold of 30 HU, thin caps can appear to be thick, and at 75 HU, thick caps can appear to be thin. In the illustrated example, at 60 HU, there is a clean separation between thin cap fibroatheromas and thick cap fibroatheromas, enabling the two to be readily distinguished from one another. It will be appreciated that FIG. 51 is merely for illustrative purposes. In practice, the actual thresholds can be different from those depicted in FIG. 51, and can depend on a variety of factors such as scan parameters, reconstruction algorithm, the particular scanner used, patient anatomy, patient physiology, etc.

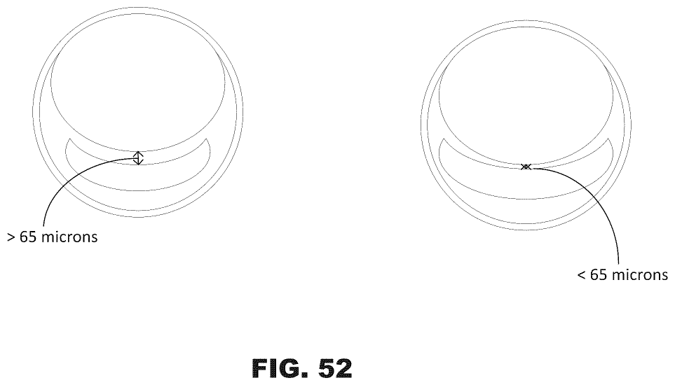

FIG. 52 is a drawing that illustrates an example of thin (<65 micrometers) and thick (>65 micrometers) cap fibroatheromas. As described herein, thin cap fibroatheromas (TCFAs) can pose a significantly greater risk than thick cap fibroatheromas (ThCFAs), as TCFAs can have a higher likelihood of rupturing.

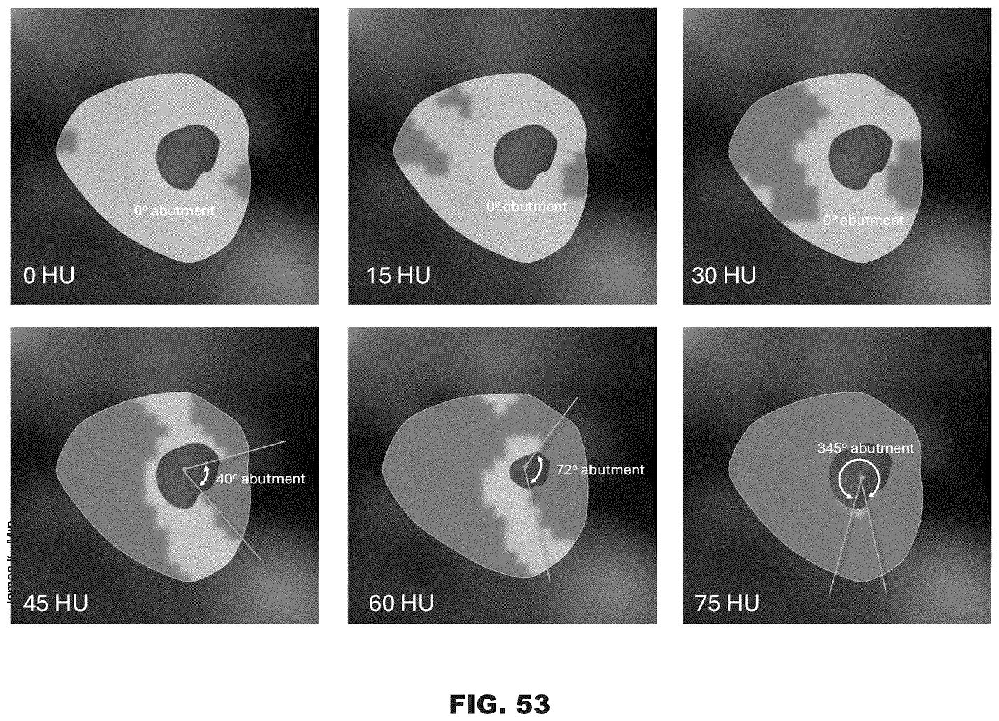

FIG. 53 is a drawing that illustrates identified LAP in CCTA images at various LAP thresholds. As shown in FIG. 53, the chosen LAP threshold can have a significant impact on whether a CCTA image appears to show TCFA or not, and if so, to what degree the TCFA is present. For example, abutment angles are shown in FIG. 53 and vary from no abutment with the lumen to nearly complete abutment with the lumen depending upon the LAP threshold. As described herein, both whether or not the LAP abuts the lumen and the degree to which the LAP abuts the lumen indicate whether TCFA is present and, if so, can indicate how vulnerable the TCFA is to rupture.

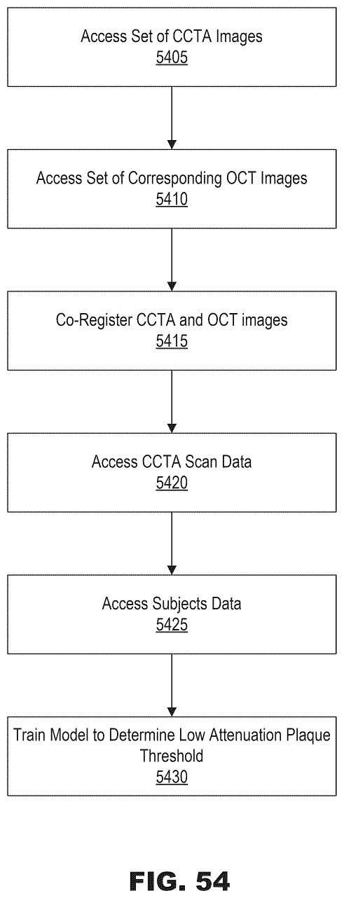

FIG. 54 is a flowchart that illustrates an example process for training a machine learning model to determine low attenuation plaque thresholds according to some embodiments. At operation 5405, a system can access a set of CCTA images. At operation 5410, the system can access a set of corresponding OCT images. At operation 5415, the system can co-register the CCTA images and their corresponding OCT images. In some embodiments, the CCTA and OCT images are already co-registered, and this operation is not performed. At operation 5420, the system can access CCTA scan data for the CCTA images. The CCTA scan data can include scan parameters, information about the reconstruction algorithm, information about the contrast agent used, information about the scanner used, and/or the like. At operation 1225, the system can access subject data for subjects depicted in the CCTA images. The subject data can include information such as gender, sex, weight, body mass index, and so forth. At operation 5430, the system can train a machine learning model to determine low attenuation plaque thresholds. For example, the model can be trained using supervised learning in which the CCTA image, scan data, and subjects data are used to form inputs and the model is trained to determine LAP thresholds based on LA P parameters (e.g., presence of LA P, extent of LAP (e.g., volume, length, area, etc.) determined from the OCT images. For example, in some embodiments, a training process can include determining a LAP threshold, determining LAP parameters using a second machine learning model, and adjusting parameters of the model based on the determined LAP parameters (e.g., based on a comparison of the LAP parameters determining using the second machine learning models and the LAP parameters determined from the OCT images). In some embodiments, the inputs can vary. For example, different embodiments may use different scan data and/or different subject data. In some embodiments, subject data is not used.

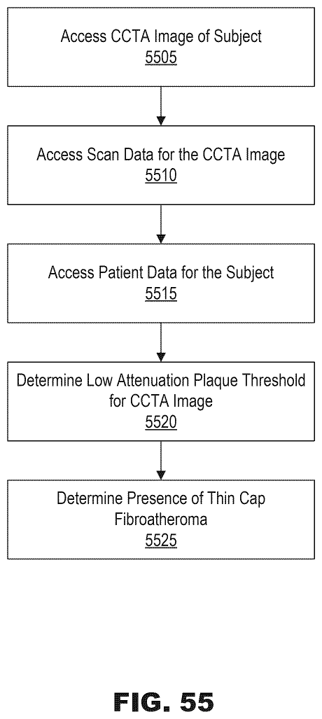

FIG. 55 is a flowchart that illustrates an example process for determining the presence and/or extent of thin cap fibroatheroma. At operation 5505, a system can access a CCTA image of a subject. At operation 5510, the system can access scan data associated with the CCTA image. At operation 5515, the system can access patient data for the subject. The patient data can include, for example, sex, weight, gender, body mass index, etc. In some embodiments, the system may not access patient data and the patient data may not be used in determining a LAP threshold. At operation 5520, the system can determine a LAP threshold for the CCTA image, for example using a machine learning model. At operation 5525, the system can set the LAP threshold and analyze the image to determine the presence of TCFA in the image. In some embodiments, a second machine learning model is used to analyze the image to determine the presence of TCFA.

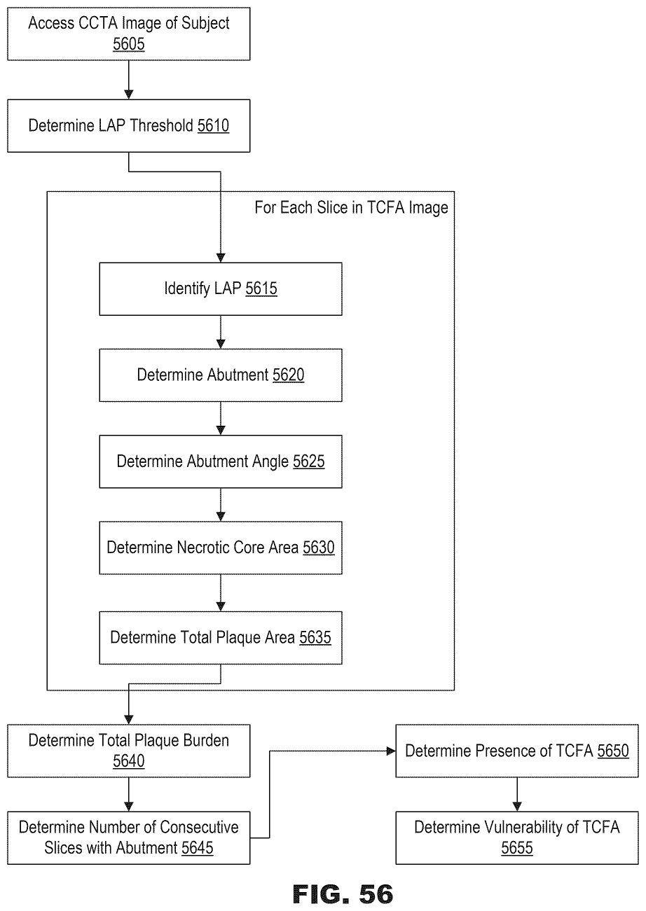

FIG. 56 is a flowchart that illustrates an example process for determining presence and vulnerability of TCFA according to some implementations. At operation 5605, a system can access a CCTA image of a subject. The CCTA image can be a 3D CCTA image. At operation 5610, the system can determine a LAP threshold for the CCTA image, for example using a lookup table or machine learning model as described herein. For one or more slices of the CCTA image (e.g., for each slice of the CCTA image), the system can identify low attenuation plaque at operation 5615 and determine abutment of the low attenuation plaque with a lumen at operation 5620. At operation 5625, the system can, for the one or more slices (e.g., for slices that show abutment), determine an abutment angle between the LAP and the lumen. At operation 5630, the system can determine a necrotic core area. At operation 5635, the system can determine a total plaque area. At operation 5640, the system can determine a total plaque burden. At operation 5645, the system can determine a number of consecutive slices with abutment. At operation 5650, the system can determine a presence of TCFA. At operation 5655, the system can determine a vulnerability of the TCFA. The vulnerability can be a numerical value, categorical value, etc. For example, if determined abutment angles are below a threshold value or the number of slices that show TCFA is below a threshold number, a vulnerability may be relatively low as such TCFA is typically less likely to rupture than TCFAs with large abutment angles and/or that extend over a long length of a vessel.

### Thin Cap Fibroatheroma Example Embodiments

Embodiment 1. A computer-implemented method for determining presence of thin-cap fibroatheroma (TCFA) using image analysis of a coronary computed tomography angiography (CCTA) image, the method comprising: accessing, by a computer system, a CCTA image of a subject, the CCTA image of the subject depicting one or more regions of one or more arteries of the subject, wherein the one or more regions comprise one or more regions of plaque; applying, to the CCTA image of the subject by the computer system, a machine learning algorithm for determining a desired low attenuation plaque threshold specific for the CCTA image of the subject, wherein the desired low attenuation plaque threshold is configured to allow an image processing algorithm to identify one or more low attenuation plaque parameters from the CCTA image of the subject at or above a predetermined accuracy level, wherein the one or more low attenuation plaque parameters identified from the CCTA are further configured to be used to determine presence of TCFA on the CCTA image, wherein the machine learning algorithm is trained by: accessing a set of sample CCTA images; accessing a set of sample optical coherence tomography (OCT) images, wherein each of the CCTA images of the set of sample CCTA images corresponds to an OCT image of the set of sample OCT images, and wherein each of the CCTA images of the set of sample CCTA images is co-registered with its corresponding OCT image of the set of sample OCT images; determining, for each CCTA image of the set of sample CCTA images, one or more low attenuation plaque parameters, wherein the one or more low attenuation plaque parameters comprise one or more of low attenuation plaque length, low attenuation plaque volume, low attenuation plaque area, or low attenuation plaque abutment angle; analyzing each OCT image of the set of sample OCT images to identify one or more regions of low attenuation plaque and presence or absence of T CFA; and training a machine learning model to determine a desired low attenuation plaque threshold for identifying the one or more low attenuation plaque parameters from CCTA images, wherein the machine learning model is trained using supervised learning to output a desired low attenuation plaque threshold based at least in part on a correlation between the one or more low attenuation plaque parameters determined from the set of sample CCTA images and presence or absence of TCFA from the set of sample OCT images; identifying, by the computer system, one or more regions of low attenuation plaque in the CCTA image using the desired low attenuation plaque threshold; identifying, by the computer system, one or more low attenuation plaque parameters from analyzing the one or more regions of low attenuation plaque identified in the CCTA image of the subject; and determining, by the computer system, the presence of TCFA in the CCTA image based at least in part on the one or more low attenuation plaque parameters, wherein the computer system comprises a computer processor and an electronic storage medium.
Embodiment 2. The computer-implemented method of claim 1, wherein the predetermined accuracy level of the one or more low attenuation plaque parameters identified from the CCTA image is determined relative to one or more low attenuation plaque parameters identified from an OCT image.
Embodiment 3. A computer-implemented method for determining a presence of thin-cap fibroatheroma (TCFA) using image analysis of a coronary computed tomography angiography (CCTA) image, the method comprising: accessing a first set of medical images, wherein the first set of medical images comprises optical coherence tomography images; accessing a second set of medical images, wherein the second set of medical images comprises CCTA images, wherein each medical image of the first set of medical images corresponds to a medical image of the second set of medical images, and wherein each medical image of the first set of medical images is co-registered with its corresponding medical image of the second set of medical images; determining, for each medical image of the first set of medical images, a low attenuation plaque parameter, wherein the low attenuation plaque parameter comprises at least one of: low attenuation plaque length, low attenuation plaque volume, low attenuation plaque area, or low attenuation plaque abutment angle; accessing a set of scan data for the second set of medical images; and training a machine learning model to determine a low attenuation plaque threshold, wherein the machine learning model is trained using supervised learning to output a low attenuation plaque threshold based on the scan data and CCTA images.
Embodiment 4. The computer-implemented method of embodiment 3, wherein training the machine learning model comprises: accessing a CCTA image and scan data corresponding to the CCTA image; providing a representation of at least a portion of the CCTA image and the scan data to the machine learning model; determining a low attenuation plaque threshold associated with the CCTA image based on an output of the machine learning model; determining, by applying an image processing algorithm to the CCTA image, a low attenuation plaque parameter; comparing the determined low attenuation plaque parameter to a corresponding low attenuation plaque parameter determined from a corresponding OCT image; and updating one or more weights of the machine learning model based on a result of the comparing.
Embodiment 5. A computer-implemented method comprising: accessing a first set of medical images, accessing a second set of medical images, wherein each medical image of the first set of medical images corresponds to a medical image of the second set of medical images, and wherein each medical image of the first set of medical images is co-registered with its corresponding medical image of the second set of medical images; determining, for each medical image of the first set of medical images, a low attenuation plaque parameter; accessing a set of scan data for the second set of medical images; and training a machine learning model to determine a low attenuation plaque threshold.
Embodiment 6. The computer-implemented method of embodiment 5, wherein the first set of medical images comprises optical coherence tomography (OCT) images.
Embodiment 7. The computer-implemented method of embodiment 5 or 6, wherein the second set of medical images comprises coronary computed tomography angiography (CCTA) images.
Embodiment 8. The computer-implemented method of any of embodiments 5-7, wherein the low attenuation plaque parameter comprises one or more of: a low attenuation plaque length, a low attenuation plaque volume, a low attenuation plaque area, or a low attenuation plaque abutment angle.
Embodiment 9. A computer-implemented method comprising: accessing a coronary computed tomography angiography image; accessing scan data associated with the coronary computed tomography angiography image; determining, using a machine learning model, the coronary computed tomography angiography image, and the scan data, a low attenuation plaque threshold; and determining, based on the coronary computed tomography angiography image, a low attenuation plaque parameter.
Embodiment 10. The computer-implemented method of embodiment 9, wherein the low attenuation plaque parameter comprises one or more of: a low attenuation plaque length, a low attenuation plaque volume, a low attenuation plaque area, or a low attenuation plaque abutment angle.
Embodiment 11. The computer-implemented method of embodiment 9 or 10, wherein the scan data comprises scan parameter data and patient parameter data.
Embodiment 12. The computer-implemented method of embodiment 11, wherein the scan parameter data comprises at least one of: peak kilovoltage or current.
Embodiment 13. The computer-implemented method of embodiment 11 or 12, wherein the patient parameter data comprises at least one of: sex, gender, height, weight, or body mass index.
Embodiment 14. The computer-implemented method any of embodiments 9-13, further comprising: determining a vulnerability associated with an identified region of low attenuation plaque, where the vulnerability indicates a risk that the low attenuation plaque will rupture.
Embodiment 15. The computer-implemented method of any of embodiments 9-14, wherein the low attenuation plaque threshold is between about 0 HU and about 75 HU.
Embodiment 16. The computer-implemented method of any of embodiments 9-15,further comprising identifying a presence of thin cap fibroatheroma.
Embodiment 17. The computer-implemented method of embodiment 16, wherein the presence of thin cap fibroatheroma is based on at least one of: an abutment angle of low attenuation plaque with a lumen, a number of consecutive slices of the coronary computed tomography angiography image showing low attenuation plaque abutting the lumen, low attenuation plaque area being more than a threshold amount of a total plaque area, total plaque burden being greater than a threshold value, or low attenuation plaque directly abutting the lumen.
Embodiment 18. A computer-implemented method comprising: accessing a first set of medical images, wherein the first set of medical images comprises optical coherence tomography images; accessing a second set of medical images, wherein the second set of medical images comprises coronary computed tomography angiography images, wherein each medical image of the first set of medical images corresponds to a medical image of the second set of medical images, and wherein each medical image of the first set of medical images is co-registered with its corresponding medical image of the second set of medical images; determining, for each medical image of the first set of medical images, a non-calcified plaque parameter, wherein the non-calcified plaque parameter comprises at least one of: non-calcified plaque length, non-calcified plaque volume, non-calcified plaque area, or non-calcified plaque abutment angle; accessing a set of scan data for the second set of medical images; and training a machine learning model to determine a non-calcified plaque threshold, wherein the machine learning model is trained using supervised learning to output a non-calcified plaque threshold based on the scan data and CCTA images.
Embodiment 19. The computer-implemented method of embodiment 18, wherein training the machine learning model comprises: accessing a CCTA image and scan data corresponding to the CCTA image; providing a representation of at least a portion of the CCTA image and the scan data to the machine learning model; determining a non-calcified plaque threshold associated with the CCTA image based on an output of the machine learning model; determining, by applying an image processing algorithm to the CCTA image, a non-calcified plaque parameter; comparing the determined non-calcified plaque parameter to a corresponding non-calcified plaque parameter determined from a corresponding OCT image; and updating one or more weights of the machine learning model based on a result of the comparing.
Embodiment 20. A computer-implemented method comprising: accessing a first set of medical images, accessing a second set of medical images, wherein each medical image of the first set of medical images corresponds to a medical image of the second set of medical images, and wherein each medical image of the first set of medical images is co-registered with its corresponding medical image of the second set of medical images; determining, for each medical image of the first set of medical images, a non-calcified plaque parameter; accessing a set of scan data for the second set of medical images; and training a machine learning model to determine a non-calcified plaque threshold.
Embodiment 21. The computer-implemented method of embodiment 20, wherein the first set of medical images comprises optical coherence tomography (OCT) images or intravascular ultrasound (IVUS) images.
Embodiment 22. The computer-implemented method of embodiment 20 or 21, wherein the second set of medical images comprises coronary computed tomography angiography (CCTA) images.
Embodiment 23. The computer-implemented method of any of embodiments 20-22, wherein the non-calcified plaque parameter comprises one or more of: a non-calcified plaque length, a non-calcified plaque volume, a non-calcified plaque area, or a non-calcified plaque abutment angle.
Embodiment 24. A computer-implemented method comprising: accessing a coronary computed tomography angiography image; accessing scan data associated with the coronary computed tomography angiography image; determining, using a machine learning model, the coronary computed tomography angiography image, and the scan data, a non-calcified plaque threshold; and determining, based on the coronary computed tomography angiography image, a non-calcified plaque parameter.
Embodiment 25. The computer-implemented method of embodiment 24, wherein the non-calcified plaque parameter comprises one or more of: a non-calcified plaque length, a non-calcified plaque volume, a non-calcified plaque area, or a non-calcified plaque abutment angle.
Embodiment 26. The computer-implemented method of embodiment 24 or 25, wherein the scan data comprises scan parameter data and patient parameter data.
Embodiment 27. The computer-implemented method of embodiment 26, wherein the scan parameter data comprises at least one of: peak kilovoltage or current.
Embodiment 28. The computer-implemented method of embodiment 26 or 27, wherein the patient parameter data comprises at least one of: sex, gender, height, weight, or body mass index.
Embodiment 29. The computer-implemented method of any of embodiments 24-28, wherein the non-calcified plaque threshold is between about 31 HU and about 350 HU.
Embodiment 30. A computer-implemented method comprising: accessing a first set of medical images, wherein the first set of medical images comprises optical coherence tomography images; accessing a second set of medical images, wherein the second set of medical images comprises coronary computed tomography angiography images, wherein each medical image of the first set of medical images corresponds to a medical image of the second set of medical images, and wherein each medical image of the first set of medical images is co-registered with its corresponding medical image of the second set of medical images; determining, for each medical image of the first set of medical images, a calcified plaque parameter, wherein the calcified plaque parameter comprises at least one of: calcified plaque length, calcified plaque volume, calcified plaque area, or calcified plaque abutment angle; accessing a set of scan data for the second set of medical images; and training a machine learning model to determine a calcified plaque threshold, wherein the machine learning model is trained using supervised learning to output a calcified plaque threshold based on the scan data and CCTA images.
Embodiment 31. The computer-implemented method of embodiment 30, wherein training the machine learning model comprises: accessing a CCTA image and scan data corresponding to the CCTA image; providing a representation of at least a portion of the CCTA image and the scan data to the machine learning model; determining a calcified plaque threshold associated with the CCTA image based on an output of the machine learning model; determining, by applying an image processing algorithm to the CCTA image, a calcified plaque parameter; comparing the determined calcified plaque parameter to a corresponding calcified plaque parameter determined from a corresponding OCT image; and updated one or more weights of the machine learning model based on a result of the comparing.
Embodiment 32. A computer-implemented method comprising: accessing a first set of medical images, accessing a second set of medical images, wherein each medical image of the first set of medical images corresponds to a medical image of the second set of medical images, and wherein each medical image of the first set of medical images is co-registered with its corresponding medical image of the second set of medical images; determining, for each medical image of the first set of medical images, a calcified plaque parameter; accessing a set of scan data for the second set of medical images; and training a machine learning model to determine a calcified plaque threshold.
Embodiment 33. The computer-implemented method of embodiment 32, wherein the first set of medical images comprises optical coherence tomography (OCT) images.
Embodiment 34. The computer-implemented method of embodiment 32 or 33, wherein the second set of medical images comprises coronary computed tomography angiography (CCTA) images.
Embodiment 35. The computer-implemented method of embodiment 32, 33, or 34, wherein the calcified plaque parameter comprises one or more of: a calcified plaque length, a calcified plaque volume, a calcified plaque area, or a calcified plaque abutment angle.
Embodiment 36. A computer-implemented method comprising: accessing a coronary computed tomography angiography image; accessing scan data associated with the coronary computed tomography angiography image; determining, using a machine learning model, the coronary computed tomography angiography image, and the scan data, a calcified plaque threshold; and determining, based on the coronary computed tomography angiography image, a calcified plaque parameter.
Embodiment 37. The computer-implemented method of embodiment 36, wherein the calcified plaque parameter comprises one or more of: a calcified plaque length, a calcified plaque volume, a calcified plaque area, or a calcified plaque abutment angle.
Embodiment 38. The computer-implemented method of embodiment 36 or 37, wherein the scan data comprises scan parameter data and patient parameter data.
Embodiment 39. The computer-implemented method of embodiment 36, 37, or 38, wherein the scan data comprises at least one of: peak kilovoltage or current.
Embodiment 40. The computer-implemented method of any of embodiments 36-39, wherein the patient parameter data comprises at least one of: sex, gender, height, weight, or body mass index.
Embodiment 41. The computer-implemented method of any of embodiments 36-40, wherein the calcified plaque threshold is between about 351 HU and about 2500 HU.

### Other Embodiment(s)

Although this invention has been disclosed in the context of certain embodiments and examples, it will be understood by those skilled in the art that the invention extends beyond the specifically disclosed embodiments to other alternative embodiments and/or uses of the invention and obvious modifications and equivalents thereof. In addition, while several variations of the embodiments of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the embodiments of the disclosed invention. Any methods disclosed herein need not be performed in the order recited. Thus, it is intended that the scope of the invention herein disclosed should not be limited by the particular embodiments described above.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment. The headings used herein are for the convenience of the reader only and are not meant to limit the scope of the inventions or claims.

Further, while the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (e.g., as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present.

1. A computer-implemented method for estimating fractional flow reserve for a subject based on image analysis of a medical image of the subject, the computer-implemented method comprising:
accessing, by a computer system, the medical image of the subject, wherein the medical image of the subject depicts one or more regions of one or more coronary arteries of the subject;
analyzing, by the computer system, the medical image of the subject to identify the one or more regions of the one or more coronary arteries;
determining, by the computer system, vessel geometry of the identified one or more regions of the one or more coronary arteries, wherein the vessel geometry is determined based at least in part on a lumen wall and a vessel wall of the identified one or more regions of the one or more coronary arteries;
generating an input for a machine learning model for determining a fluid flow characteristic, the input based at least in part on the determined vessel geometry;
determining, by the computer system using the machine learning model, the fluid flow characteristic of the identified one or more regions of the one or more coronary arteries, wherein the machine learning model is trained to determine the fluid flow characteristic using fluid flow measurements collected from a plurality of three-dimensional (3D) printed models of coronary arteries of a plurality of sample subjects, and wherein the machine learning model is trained to determine the fluid flow characteristic by:
for each sample medical image of a plurality of sample medical images associated with a plurality of sample subjects:
accessing the sample medical image;
analyzing the sample medical image to identify a plurality of vessels depicted in the sample medical image;
determining a geometry of each vessel of the plurality of vessels;
generating a 3D-printable model of the plurality of vessels, wherein the 3D-printable model comprises a file stored in a non-transitory computer-readable medium; and
accessing pressure measurements collected from a physical 3D model of the plurality of vessels produced by a 3D printer using the 3D-printable model,
wherein the pressure measurements are collected by positioning a pressure sensor in a lumen of the physical 3D model at a plurality of locations within the physical 3D model,
wherein the pressure measurements are collected at a plurality of inlet pressures, and
wherein the pressure measurements are collected at a plurality of fluid flow rates; and
providing the determined geometries and the pressure measurements to the machine learning model, wherein the determined geometries are used as inputs, and wherein the determined pressure measurements are used as outputs,
wherein the machine learning model is trained to output a pressure pullback gradient curve;
determining, by the computer system using the determined fluid flow characteristic of the identified one or more regions of the one or more coronary arteries, one or more fractional flow reserve values associated with one or more locations within the one or more regions of the one or more coronary arteries of the subject,
wherein the computer system comprises a computer processor and an electronic storage medium.

2. A computer-implemented method for estimating fractional flow reserve for a subject based on image analysis of a medical image of the subject, the computer-implemented method comprising:
accessing, by a computer system, the medical image of the subject, wherein the medical image of the subject depicts one or more regions of one or more coronary arteries of the subject;
analyzing, by the computer system, the medical image of the subject to identify the one or more regions of the one or more coronary arteries;
determining, by the computer system, vessel geometry of the identified one or more regions of the one or more coronary arteries, wherein the vessel geometry is determined based at least in part on a lumen wall and a vessel wall of the identified one or more regions of the one or more coronary arteries;
generating an input for a machine learning model for determining a fluid flow characteristic, the input based at least in part on the determined vessel geometry;
determining, by the computer system using the machine learning model, the fluid flow characteristic of the identified one or more regions of the one or more coronary arteries, wherein the machine learning model is trained to determine the fluid flow characteristic using fluid flow measurements collected from a plurality of three-dimensional (3D) printed models of coronary arteries of a plurality of sample subjects; and
determining, by the computer system using the determined fluid flow characteristic of the identified one or more regions of the one or more coronary arteries, one or more fractional flow reserve values associated with one or more locations within the one or more regions of the one or more coronary arteries of the subject,
wherein the computer system comprises a computer processor and an electronic storage medium.

3. The computer-implemented method of claim 2, wherein the machine learning model is trained to determine the fluid flow characteristic by:
for each sample medical image of a plurality of sample medical images associated with a plurality of sample subjects:
accessing the sample medical image;
analyzing the sample medical image to identify a plurality of vessels depicted in the sample medical image;
determining a geometry of each vessel of the plurality of vessels;
generating a 3D-printable model of the plurality of vessels, wherein the 3D-printable model comprises a file stored in a non-transitory computer-readable medium; and
accessing pressure measurements collected from a physical 3D model of the plurality of vessels produced by a 3D printer using the 3D-printable model,
wherein the pressure measurements are collected by positioning a pressure sensor in a lumen of the physical 3D model at a plurality of locations within the physical 3D model,
wherein the pressure measurements are collected at a plurality of inlet pressures, and
wherein the pressure measurements are collected at a plurality of fluid flow rates; and
providing the determined geometries and the pressure measurements to the machine learning model, wherein the determined geometries are used as inputs and wherein the determined pressure measurements are used as outputs,
wherein the machine learning model is trained to output the pressure measurements.

4. The computer-implemented method of claim 2, wherein the machine learning model is trained to output a pressure pullback gradient curve.

5. The computer-implemented method of claim 2, wherein the input includes information about a region of plaque detected in the one or regions of the one or more coronary arteries of the subject, wherein the information about the region of plaque includes one or more of: plaque length, plaque area, plaque volume, or plaque density.

6. The computer-implemented method of claim 5, wherein the plaque density is one or more of: low density non-calcified plaque, non-calcified plaque, or calcified plaque.

7. The computer-implemented method of claim 6, wherein the medical image is a coronary computed tomography angiography image,
wherein low density non-calcified plaque corresponds to a radiodensity of between about −189 Hounsfield units (HU) and about 30 HU,
wherein non-calcified plaque corresponds to a radiodensity of between about 31 HU and about 350 HU, and
wherein calcified plaque corresponds to a radiodensity of between about 351 HU and about 2500 HU.

8. The computer-implemented method of claim 2, wherein the medical image is a coronary computed tomography angiography image.

9. The computer-implemented method of claim 2, wherein the wherein the one or more coronary arteries comprise one or more of left main (LM), ramus intermedius (RI), left anterior descending (LA D), diagonal one (D1), diagonal two (D2), left circumflex (Cx), obtuse marginal one (OM1), obtuse marginal two (OM2), left posterior descending (L-PDA), left posterolateral branch (L-PLB), right coronary (RCA), right posterior descending (R-PDA), or right posterolateral branch (R-PLB).

10. The computer-implemented method of claim 3, wherein at least one physical 3D model is printed using a plurality of materials, wherein at least one material of the plurality of materials is selected based on a density of a region of plaque identified in the sample medical image.

11. The computer-implemented method of claim 10, wherein the density of the region of plaque is determined based on a radiodensity of the region of plaque in the sample medical image, wherein the density is at least one of: low density non-calcified plaque, non-calcified plaque, or calcified plaque.

12. The computer-implemented method of claim 2, wherein a fractional flow reserve value of the one or more fractional flow reserve values is determined as a ratio of pressure di stal to a region of plaque and pressure proximal to the region of plaque.

13. The computer-implemented method of claim 2, wherein the one or more fractional flow reserve values is determined for at least one of: a vessel, a vessel segment, or a vessel unit length.

14. A system for estimating fractional flow reserve for a subject based on image analysis of a medical image of the subject, the system comprising:
at least one processor; and
a computer-readable medium storing instructions that, when executed by the system, cause the system to:
access the medical image of the subject, wherein the medical image of the subject depicts one or more regions of one or more coronary arteries of the subject;
analyze the medical image of the subject to identify the one or more regions of the one or more coronary arteries;
determine vessel geometry of the identified one or more regions of the one or more coronary arteries, wherein the vessel geometry is determined based at least in part on a lumen wall and a vessel wall of the identified one or more regions of the one or more coronary arteries;
generate an input for a machine learning model for determining a fluid flow characteristic, the input based at least in part on the determined vessel geometry;
determine, using the machine learning model, the fluid flow characteristic of the identified one or more regions of the one or more coronary arteries, wherein the machine learning model is trained to determine the fluid flow characteristic using fluid flow measurements collected from a plurality of three-dimensional (3D) printed models of coronary arteries of a plurality of sample subjects; and
determine, using the determined fluid flow characteristic of the identified one or more regions of the one or more coronary arteries, one or more fractional flow reserve values associated with one or more locations within the one or more regions of the one or more coronary arteries of the subject.

15. The system of claim 14, wherein the machine learning model is trained to determine the fluid flow characteristic by:
for each sample medical image of a plurality of sample medical images associated with a plurality of sample subjects:
accessing the sample medical image;
analyzing the sample medical image to identify a plurality of vessels depicted in the sample medical image;
determining a geometry of each vessel of the plurality of vessels;
generating a 3D-printable model of the plurality of vessels, wherein the 3D-printable model comprises a file stored in a non-transitory computer-readable medium; and
accessing pressure measurements collected from a physical 3D model of the plurality of vessels produced by a 3D printer using the 3D-printable model,
wherein the pressure measurements are collected by positioning a pressure sensor in a lumen of the physical 3D model at a plurality of locations within the physical 3D model,
wherein the pressure measurements are collected at a plurality of inlet pressures, and
wherein the pressure measurements are collected at a plurality of fluid flow rates;
providing the determined geometries and the pressure measurements to the machine learning model, wherein the determined geometries are used as inputs and wherein the determined pressure measurements are used as outputs,
wherein the machine learning model is trained to output the pressure measurements.

16. The system of claim 14, wherein the machine learning model is trained to output a pressure pullback gradient curve.

17. The system of claim 14, wherein the input includes information about a region of plaque detected in the one or regions of the one or more coronary arteries of the subject, wherein the information about the region of plaque includes one or more of: plaque length, plaque area, plaque volume, or plaque density.

18. The system of claim 17, wherein the plaque density is one or more of: low density non-calcified plaque, non-calcified plaque, or calcified plaque.

19. The system of claim 18, wherein the medical image is a coronary computed tomography angiography image,
wherein low density non-calcified plaque corresponds to a radiodensity of between about −189 Hounsfield units (HU) and about 30 HU,
wherein non-calcified plaque corresponds to a radiodensity of between about 31 HU and about 350 HU, and
wherein calcified plaque corresponds to a radiodensity of between about 351 HU and about 2500 HU.

20. The system of claim 14, wherein the medical image is a coronary computed tomography angiography image.

21. The system of claim 14, wherein the wherein the one or more coronary arteries comprise one or more of left main (LM), ramus intermedius (RI), left anterior descending (LAD), diagonal one (D1), diagonal two (D2), left circumflex (Cx), obtuse marginal one (OM1), obtuse marginal two (OM2), left posterior descending (L-PDA), left posterolateral branch (L-PLB), right coronary (RCA), right posterior descending (R-PDA), or right posterolateral branch (R-PLB).

22. The system of claim 15, wherein at least one physical 3D model is printed using a plurality of materials, wherein at least one material of the plurality of materials is selected based on a density of a region of plaque identified in the sample medical image.

23. The system of claim 22, wherein the density of the region of plaque is determined based on a radiodensity of the region of plaque in the sample medical image, wherein the density is at least one of: low density non-calcified plaque, non-calcified plaque, or calcified plaque.

24. The system of claim 14, wherein a fractional flow reserve value of the one or more fractional flow reserve values is determined as a ratio of pressure distal to a region of plaque and pressure proximal to the region of plaque.

25. The system of claim 14, wherein the one or more fractional flow reserve values is determined for at least one of: a vessel, a vessel segment, or a vessel unit length.
